(19) United States
(12) Patent Application Publication
(10) Pub. No.: US 2017/0165077 A1
(43) Pub. Date: Jun. 15, 2017

(54) RETENTION DEVICES, LATTICES AND RELATED SYSTEMS AND METHODS

(71) Applicant: WOVEN ORTHOPEDIC TECHNOLOGIES, LLC, Manchester, CT (US)

(72) Inventor: Christopher McDonnell, Sandy Hook, CT (US)

(73) Assignee: WOVEN ORTHOPEDIC TECHNOLOGIES, LLC, Manchester, CT (US)

(21) Appl. No.: 15/374,773

(22) Filed: Dec. 9, 2016

Related U.S. Application Data
(60) Provisional application No. 62/265,220, filed on Dec. 9, 2015.
(60) Provisional application No. 62/265,236, filed on Dec. 9, 2015.
(60) Provisional application No. 62/265,251, filed on Dec. 9, 2015.
(60) Provisional application No. 62/265,275, filed on Dec. 9, 2015.
(60) Provisional application No. 62/265,276, filed on Dec. 9, 2015.
(60) Provisional application No. 62/265,279, filed on Dec. 9, 2015.

(51) Int. Cl. A61F 2/30; A61B 17/70

(52) U.S. Cl. CPC A61F 2/30907; A61B 17/7001; A61B 2017/8655

(57) ABSTRACT

A woven retention device, lattice device and woven patch device that are configured to receive a fastener in a bone hole can be configured to impede biofilm formation. The devices can be made of woven filaments that outline apertures of varying sizes and shapes and can serve as an interface between a fastener and the bone material. The devices can be configured to allow for optimal bone growth while at the same time minimizing the likelihood that biofilm forms thereon. The devices can be made of materials that facilitate soft tissue fixation, and screw-activated expansion.

### CROSS REFERENCE TO RELATED APPLICATIONS

This application claims priority to U.S. Provisional Application No. 62/265,220, filed Dec. 9, 2015, U.S. Provisional Application No. 62/265,279, filed Dec. 9, 2015, U.S. Provisional Application No. 62/265,236, filed Dec. 9, 2015, U.S. Provisional Application No. 62/265,251, filed Dec. 9, 2015, U.S. Provisional Application No. 62/265,276, filed Dec. 9, 2015, and U.S. Provisional Application No. 62/265,275, filed Dec. 9, 2015, the contents of which are hereby incorporated herein by reference in their entireties.

### TECHNICAL FIELD

The present invention relates to devices, systems and methods for use in fixing fasteners to bone tissue.

### BACKGROUND

In orthopedic surgery it is common to secure a bone screw to a patient's bone. Bone fracture repair is surgery to fix a broken bone using plates, nails, screws, or pins. It is common in the treatment of fractures to attach a plate to the bone utilizing bone screws. The resulting construct prevents motion of the fractured bone so that the bone can heal. Alternatively, one or more screws may be inserted across the break to hold it in place.

In the treatment of spinal disorders, pedicle screws are inserted into the patient's vertebrae to serve as anchor points that can then be connected with a rod. This construct prevents motion of the vertebral segments that are to be fused.

In the treatment of detached tendons, screw-like tissue anchors are inserted into the patient's bone to serve as an anchor for the reattachment of the tendon.

One complication with the use of bone screws is the loss of fixation or grip between the bone screw and the patient's bone. Another complication with the use of bone screws is the stripping of the hole in the bone when the bone screw is inserted. This results in the loss of purchase and holding strength of the bone screw.

The presence of osteoporotic bone can increase the likelihood of complications by reducing the purchase or grip of the bone screw to the patient's bone, resulting in a loss of holding strength and loosening of the bone screw or pullout of the bone screw.

Infections deep inside bone require systemic antibacterial treatments, which disrupt entire systems.

Cellular responses and micro-organisms that create biofilms prevent bony ingrowth.

A woven patch can be used in orthopedics. Currently, commercial applications use mesh, for example, to secure the lower, tibial end of a soft tissue ACL graft. In this sleeve, as described in the GTS Sleeve document, two of the lumens hold graft tissue and the third lumen accepts the GTS tapered fixation screw. See GTS Sleeve document. Another commercial application, such as the opti-mesh 3-d deployable mesh pouch (spinology), is used in intervertebral space by containing bone material and restoring the height of vertebrae. In essence, this is a cement restrictor with a pouch filled with cement. Third, other fiber or suture-based technologies are non-mesh. Pedicle shields have also been used with a semi-circular surface that are implanted within the pedicle to protect the spinal canal.

Current solutions to secure bone screws have not adequately addressed screw failure and the underlying causes of screw failure. Current solutions have also not facilitated bone healing through woven patches.

### SUMMARY

A woven patch for interfacing with a bone surface can include: a sleeve body comprising a plurality of sets of interwoven filaments that form a two-dimensional lattice with a plurality of protuberances distributed on an interior surface and an exterior surface of the lattice at a predetermined spatial relationship, the plurality of sets of interwoven monofilaments having a plurality of different diameters, the sleeve body being configured to surround at least a portion of a fastener; a first end that is configured to interface with at least a portion of the fastener; and a second end that is opposite of the first end to the sleeve body.

In a first state, the sleeve body has a plurality of combinations of filament cross-section geometries at intersection points of the interwoven filaments, the plurality of combinations of filament cross-section geometries forming a plurality of protuberance thicknesses, a thickness of each protuberance being measured in a direction as a thickness of the sleeve body. In a second state when a fastener is inserted into or applied to the lattice, pressure from the fastener is transmitted to the lattice such that the spatial relationship of the protuberances changes according to a function of bone density and according to a function of an interfacing surface shape of the fastener.

The interwoven filaments can extend across the lattice at an angle of about 45 degrees with respect to a length of the woven patch. The distributed protuberances can be arranged in a diamond-shaped pattern grid. A length of the sleeve body is in a range from about 10 mm to 100 mm.

A woven retention device for interfacing with a bone surface can include: a sleeve body comprising a plurality of filaments forming a substantially tubular lattice with a plurality of protuberances distributed on an interior surface and an exterior surface of the tubular lattice at a predetermined spatial relationship, the sleeve body being configured to surround at least a portion of a fastener, each of the plurality of protuberances being formed by an intersection point of two or more of the plurality of filaments that outline a plurality of apertures, the sleeve body comprising an orthopedic biomaterial; a proximal end that is proximal to the sleeve body and that is configured to receive at least a portion of the fastener; and a distal end that is distal to the sleeve body.

In a first state, the sleeve body can have a plurality of combinations of filament cross-section geometries at the intersection points, the plurality of combinations of filament cross-section geometries forming a plurality of protuberance thicknesses, a thickness of each protuberance being measured in a radial direction of the sleeve body. In a second state when a fastener is inserted into the tubular lattice, pressure from the fastener can be transmitted to the tubular lattice such that the spatial relationship of the protuberances changes according to a function of bone density and according to a function of an interfacing surface shape of the fastener.

The sleeve body can be configured to expand. The woven retention device can further comprise a screw-activated device including i) a screw having a threaded portion and ii) a bolt that is configured to be threaded along the threaded portion of the screw.

A first end of the screw can be attached to the distal end of the woven retention device and at least a portion of the threaded portion runs along a longitudinal direction of the body inside the woven retention device, and a second end of the screw can be configured to accept the bolt such that when the bolt is moved inside the tubular structure, a compressive force is exerted on the woven retention device by the bolt in a direction parallel to a longitudinal axis of the tubular structure.

The compressive force radially can expand the tubular structure to the expanded state. When the fastener is inserted a predetermined distance into the tubular structure, the proximal end of the woven retention device can be configured to detach from the fastener.

The sleeve body can be configured to impede biofilm formation. The biomaterial can be made of a material that impedes biofilm formation. The sleeve body can have a structure that impedes biofilm formation. The sleeve body can be configured to receive a portion of the soft tissue and the sleeve body is configured to impede biofilm formation surrounding the soft tissue. The sleeve body can comprise a coating on the plurality of filaments, wherein the coating comprises an orthopedic biomaterial. The plurality of filaments can comprise the orthopedic biomaterial. The orthopedic biomaterial can include one of PLA, PGA, PLLA, PET, PEEK, PEKK, polypropylene, polyamides, PTFE, calcium phosphate, platinum, cobalt chrome, nitinol, stainless steel, titanium, PEEK, silk and collagen, bioceramics, aluminum oxide, calcium phosphate, hydroxyapatite, glass ceramics, or any combination thereof.

A lattice for interfacing with a bone surface can comprise: a sleeve body comprising a plurality of filaments that form a substantially tubular lattice with a plurality of protuberances distributed on an interior surface and an exterior surface of the tubular lattice at a predetermined spatial relationship, the plurality of filaments having a plurality of different filament diameters; a proximal end that is proximal to the sleeve body and that is configured to receive at least one of a fastener and at least a portion of a soft tissue; and a distal end that is distal to the sleeve body.

The sleeve body can have a plurality of combinations of filament cross-section geometries at intersection points of the interwoven filaments, the plurality of combinations of filament cross-section geometries forming a plurality of different protuberance thicknesses, a thickness of each protuberance being measured in a radial direction of the sleeve body. In an implanted state of the woven retention device, the tubular lattice can be configured to interface with both the soft tissue and the bone surface to secure the soft tissue to the bone surface, the spatial relationship of the protuberances changing according to a function of bone density.

The sleeve body can be configured to receive a portion of the soft tissue. The lattice can further include an anchoring device that is configured to apply pressure to one or more regions of the soft tissue, the sleeve body distributing the applied pressure through the soft tissue and the bone surface.

The anchoring device can penetrate the soft tissue and protrude into the bone surface. The filaments can be interwoven filaments and the interwoven filaments comprise at least one set of filament that is a felted filament. The sleeve body can comprise felted filaments.

The sleeve body can be configured to receive a tendon. The sleeve body can comprise an orthopedic biomaterial, the sleeve body being configured to minimize biofilm formation on the bone and/or soft tissue.

A non-transitory computer-readable storage medium having data thereon representing a three-dimensional model suitable for use in manufacturing a three-dimensional retention device for interfacing with a bone surface can, when executed by at least one processor, cause a computing system use the data in forming the three-dimensional retention device to create a plurality of filaments having input regions that interlace with other filaments. The retention device can include: a sleeve body comprising a plurality of filaments forming a substantially tubular lattice with a plurality of protuberances distributed on an interior surface and an exterior surface of the tubular lattice at a predetermined spatial relationship, the sleeve body being configured to surround at least a portion of a fastener, each of the plurality of protuberances being formed by an intersection point of two or more of the plurality of filaments, the sleeve body including an orthopedic biomaterial; a proximal end that is proximal to the sleeve body and that is configured to receive at least a portion of the fastener; and a distal end that is distal to the sleeve body.

In a first state, the sleeve body can have a plurality of combinations of filament cross-section geometries at the intersection points, the plurality of combinations of filament cross-section geometries forming a plurality of protuberance thicknesses, a thickness of each protuberance being measured in a radial direction of the sleeve body, and in a second state when a fastener is inserted into the tubular lattice, pressure from the fastener is transmitted to the tubular lattice such that the spatial relationship of the protuberances changes according to a function of bone density and according to a function of an interfacing surface shape of the fastener.

Additional features, advantages, and embodiments of the invention are set forth or apparent from consideration of the following detailed description, drawings and claims. Moreover, it is to be understood that both the foregoing summary of the invention and the following detailed description are exemplary and intended to provide further explanation without limiting the scope of the invention as claimed.

### BRIEF DESCRIPTION OF DRAWINGS

FIG. 1A shows a perspective view of a screw, an implantable retention device and a bone, according to an embodiment of the present invention.

FIG. 1B shows a screw and an implantable retention device fixed inside a bone hole, according to an embodiment of the present invention.

FIG. 1C shows a perspective view of a screw, a woven patch and a bone, according to an embodiment of the present invention.

FIG. 1D shows a screw and a woven patch fixed inside a bone hole, according to an embodiment of the present invention.

FIG. 2A shows a schematic cross-section view of a bone hole with an implantable retention device to be inserted, according to an embodiment of the present invention.

FIG. 2B shows a schematic cross-section side view of an implantable retention device inside a bone hole and a fastener outside the bone hole to be inserted, according to an embodiment of the present invention.

FIGS. 2C and 2D show schematic side cross-section views of a fastener inserted into a bone hole and within an implantable retention device where the bone has a different density in each of FIGS. 2C and 2D, according to an embodiment of the present invention.

FIGS. 2E and 2F show schematic side cross-section views of a fastener inserted into a bone hole and within a woven patch where the bone has a different density in each of FIGS. 2E and 2F, according to an embodiment of the present invention.

FIG. 3A shows a schematic longitudinal cross-section view of an implantable retention device in a bone hole, according to an embodiment of the present invention.

FIG. 3B shows the schematic longitudinal cross-section view of an implantable retention device in a bone hole along with an inserted screw, according to an embodiment of the present invention.

FIG. 3C shows interaction forces between a screw, an implantable retention device and a bone, according to an embodiment of the present invention.

FIG. 3D shows a schematic axial view of a fastener in an implantable retention device along with resulting pressures, according to an embodiment of the present invention.

FIG. 3E shows a schematic longitudinal cross-section view of a woven patch in a bone hole, according to an embodiment of the present invention.

FIG. 3F shows the schematic longitudinal cross-section view of a woven patch in a bone hole along with an inserted screw, according to an embodiment of the present invention.

FIG. 3G shows interaction forces between a screw, a woven patch and a bone, according to an embodiment of the present invention.

FIG. 3H shows a schematic axial view of a fastener in a woven patch along with resulting pressures, according to an embodiment of the present invention.

FIG. 4A shows forces in a longitudinal direction of an implantable retention device, according to an embodiment of the present invention.

FIG. 4B shows forces in a longitudinal direction of a woven patch, according to an embodiment of the present invention.

FIG. 5A shows an illustration of an implantable retention device, according to an embodiment of the present invention.

FIG. 5B shows a slanted sectional slice of FIG. 5A.

FIG. 5C shows an illustration of a woven patch, according to an embodiment of the present invention.

FIG. 5D shows a slanted sectional slice of FIG. 5C.

FIG. 6 shows an illustration of an implantable retention device having a tapered end, according to an embodiment of the present invention.

FIG. 7A shows a two-over/two-under monofilament/monofilament weave of an implantable retention device, according to an embodiment of the present invention.

FIG. 7B shows a two-over/two-under monofilament/monofilament weave of a woven patch, according to an embodiment of the present invention.

FIG. 8A shows an implantable retention device with a tapered end along its longitudinal axis, according to an embodiment of the present invention.

FIG. 8B shows a woven patch with a tapered end along its longitudinal axis, according to an embodiment of the present invention.

FIG. 9A shows a close-up view of a portion of the implantable retention device shown in FIG. 8A.

FIG. 9B shows a close-up view of a portion of the woven patch shown in FIG. 8B.

FIG. 10A shows a cross-sectional view along line A-A in FIG. 8A of the implantable retention device.

FIG. 10B shows a cross-sectional view along line B-B in FIG. 8A of the implantable retention device.

FIG. 10C shows a cross-sectional view along line A-A in FIG. 8B of the woven patch.

FIG. 10D shows a cross-sectional view along line B-B in FIG. 8B of the woven patch.

FIG. 11A shows an end view of the implantable retention device of FIG. 8A as seen from a non-tapered end view.

FIG. 11B shows an axial view of the implantable retention device of FIG. 8A as seen from a tapered end view.

FIG. 12A shows a tapered end of an implantable retention device having a closed end, according to an embodiment of the present invention.

FIG. 12B shows a distal end view of the closed end of the implantable retention device shown in FIG. 12A.

FIG. 13A shows a section of a woven retention device, illustrating a representative fiber angle, according to an embodiment of the present invention.

FIG. 13B shows a section of a woven patch, illustrating a representative fiber angle, according to an embodiment of the present invention.

FIG. 14A shows a section of an implantable retention device, illustrating another representative fiber angle, according to an embodiment of the present invention.

FIG. 14B shows a section of a woven patch, illustrating another representative fiber angle, according to an embodiment of the present invention.

FIG. 15A shows a section of an implantable retention device illustrating multiple locations and/or points of contact on an exterior surface of the implantable retention device, according to an embodiment of the present invention.

FIG. 15B shows a section of a woven patch illustrating multiple locations and/or points of contact on an exterior surface of the implantable retention device, according to an embodiment of the present invention.

FIG. 16A shows a representation of an implantable retention device with a force or pressure applied to a location and/or point on an inside of the retention device, according to an embodiment of the present invention.

FIG. 16B shows a view of a region of an interior surface of the implantable retention device at the point shown in FIG. 16A.

FIG. 16C shows a view of a region of an exterior surface of the implantable retention device surface at the point shown on FIG. 16A.

FIG. 16D shows a representation of a woven patch with a force or pressure applied to a location and/or point on an inside of the retention device, according to an embodiment of the present invention.

FIGS. 17A and 17B show perspective views of two implantable retention devices each having different lengths, according to embodiments of the present invention.

FIGS. 17C and 17D show two implantable retention devices each having different lengths, according to embodiments of the present invention.

FIGS. 17E, 17F and 17G show an implantable retention device in a relaxed state, in a stretched state, and in an implanted state, respectively, according to embodiments of the present invention.

FIGS. 17H, 17I and 17J show an implantable retention device used with a screw-activating device in a relaxed state, in a stretched state, and in an implanted state, respectively, according to embodiments of the present invention.

FIG. 18 shows an exploded view of a screw, an implantable retention device and a pedicle hole, according to an embodiment of the present invention.

FIG. 19A shows a close-up perspective view of an exterior surface of the implantable retention device according to an embodiment of the present invention.

FIG. 19B shows a close-up perspective view of an exterior surface of the woven patch according to an embodiment of the present invention.

FIG. 20A shows a perspective view of a section of an implantable retention device having differently-shaped diameters of monofilaments according to an embodiment of the present invention.

FIG. 20B shows a perspective view of a section of a woven patch having differently-shaped diameters of monofilaments according to an embodiment of the present invention.

FIG. 21A shows an implantable retention device having differently-shaped diameters of monofilaments according to an embodiment of the present invention.

FIG. 21B shows a woven patch having differently-shaped diameters of monofilaments according to an embodiment of the present invention.

FIG. 22 shows a flow diagram of a method of utilizing an implantable retention device in an embodiment, in accordance with the principles of the present invention.

FIG. 23 shows a pullout strength comparison for a screw, a screw in a stripped bone hole, and a woven retention device with screw in a stripped bone hole, according to an example of an embodiment of the present invention.

FIG. 24 shows a pullout force versus hole diameter for a screw and a screw with a woven retention device, in accordance with the principles of the present invention.

FIG. 25 shows another pullout force versus hole diameter for a screw and a screw with a woven retention device, in accordance with the principles of the present invention.

FIG. 26 shows another pullout force versus hole diameter for a screw and a screw with a woven retention device, in accordance with the principles of the present invention.

FIG. 27 shows pullout forces measured for woven retention devices of varying construction pulled from a first material, according to examples of embodiments of the present invention.

FIG. 28 shows pullout forces measured for woven retention devices of varying construction pulled from a second material, according to examples of embodiments of the present invention.

FIG. 29 shows a first view of bones with soft tissue that can be repaired or attached according to embodiments of the present invention.

FIG. 30 shows a second view of the bone and soft tissue in FIG. 29.

FIG. 31 shows the soft tissue within the bones of FIG. 29 and a bone hole in the bones used in accordance with embodiments of the present invention.

FIG. 32 shows a partial cross-section view of a bone with a fastener, woven retention device, and soft tissue inserted within a bone hole of the bone, according to embodiments of the present invention.

FIG. 33 shows a partial see-through view of a fastener, woven retention device, and soft tissue within a bone hole, according to an embodiment of the present invention.

FIG. 34 shows a cross-section view of a fastener, woven retention device, and soft tissue within a bone hole, according to an embodiment of the present invention.

FIG. 35 shows a cross-section view of a fastener, a woven retention device, and multiple portions of soft tissue in a bone hole, according to an embodiment of the present invention.

FIG. 36 shows a partial see-through view of a woven retention device and soft tissue within a bone hole and an anchoring device according to an embodiment of the present invention.

FIG. 37 shows a partial see-through view of a woven retention device and soft tissue within a bone hole and an anchoring device according to an embodiment of the present invention.

Additional features, advantages, and embodiments of the invention are set forth or apparent from consideration of the following detailed description, drawings and claims. Moreover, it is to be understood that both the foregoing summary of the invention and the following detailed description are exemplary and intended to provide further explanation without limiting the scope of the invention as claimed.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

The devices, systems and methods described herein may be used in the area of orthopedics and, in particular, orthopedic repairs. These include various devices, systems and methods directed to fixing and/or retaining fasteners in orthopedic applications. Fixing or retaining fasteners to bone tissue is complicated by the underlining bone tissue. Understanding that an underlying cause of failure with internal fixation in bone tissue is the bone, the devices, systems and methods described herein provide for solutions that address the implant site. At the implant site, the hole and the bone benefit from an enhanced interface.

The fixation and/or retention devices, systems and methods described herein maximize fixation and/or retention in the bone tissue, including, osteoporotic bone, bone of a poor quality, and mechanically poor bone in addition to healthy bone tissue. The fixation and/or retention devices, systems and methods described herein may be used with any type of fixation including, any types of screws.

The devices, systems and methods described herein enhance the interaction of a fastener, such as a bone anchor, to a bone hole to provide enhanced fixation. Additionally, the devices, systems and methods may repair the surface of the bone hole following damage to the bone hole as in the case of stripping of the hole in the bone when a bone screw is over-tightened. Also, the devices, systems and methods provide for an enhanced bone hole surface for the reattachment of tendons in, for example, anterior/posterior cruciate ligament repair procedures, rotator cuff repair procedures, etc. The devices enhance the surface of a bone hole to enhance fixation of a bone anchor to bone and permits bone ingrowth into its structure. The devices enhance the interaction between the surface of a bone hole and the fixation device. The devices interdigitate with the bony structure and interact with the fixation device. The device alone, as a single device, enhances the surface of a bone hole to enhance fixation of a bone anchor to bone and accommodates variations in the diameter and depth of the bone hole. The devices, systems and methods can enhance fixation without requiring the use of cement and/or adhesives.

The retention devices, lattices, fixation sleeves and/or patches, systems and methods described herein maximize fixation and/or retention in the bone tissue, including, osteoporotic bone, bone of a poor quality, and mechanically poor bone in addition to healthy bone tissue. The fixation sleeve and/or patches, systems and methods described herein may be used with any type of fixation including, any types of screws.

The devices, systems and methods described herein can support a bone structure. In one embodiment, the devices, systems and methods can enhance the interaction of a bone anchor, such as a screw, a nail or a bone dowel, to a bone hole to provide enhanced fixation. Additionally, the devices, systems and methods may repair the exterior or interior surface of the bone following damage to the bone as in the case of stripping of the bone when a bone screw is over-tightened. Also, the devices, systems and methods provide for an enhanced bone surface for the reattachment of tendons in, for example, anterior/posterior cruciate ligament repair procedures, rotator cuff repair procedures, etc. The devices can enhance the surface of a bone to enhance fixation of a bone anchor to bone and can permit bone ingrowth into its structure. The devices can enhance the interaction between the surface of the bone and the fixation device. The devices can interdigitate with the bony structure and interact with the fixation device. The device alone, as a single device, enhances the surface of a bone hole to enhance fixation of a bone anchor to bone and accommodates variations in the diameter and depth of the bone hole. In one embodiment, the devices, systems and methods can enhance fixation without requiring the use of cement and/or adhesives. In another embodiment, bone cement can be applied to the surface of the bone and the patch to provide a passive patch.

Reference to a woven retention device is meant to include an implantable woven patch or implantable retention device such as a sleeve. Various embodiments described are meant to be interchangeably used with each other.

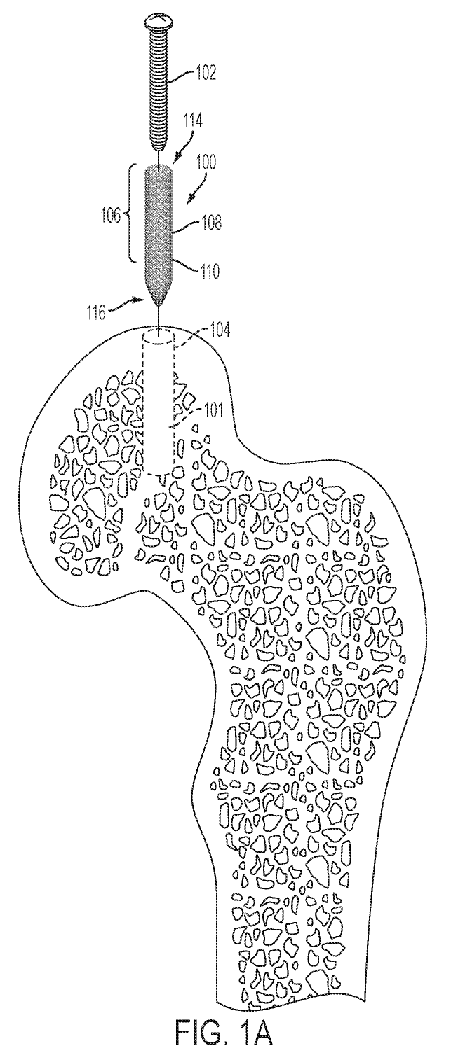

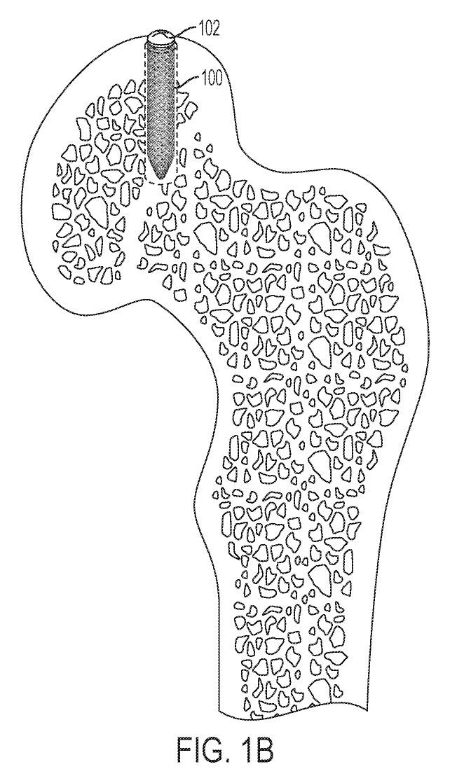

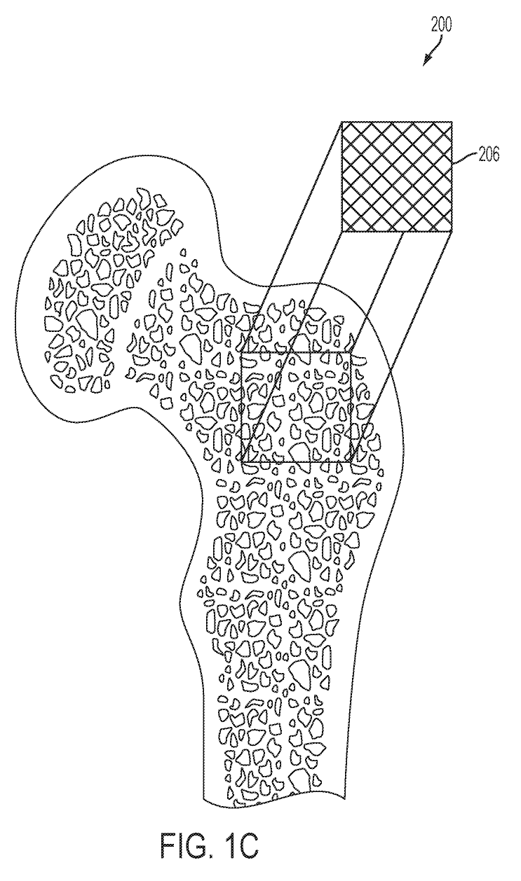

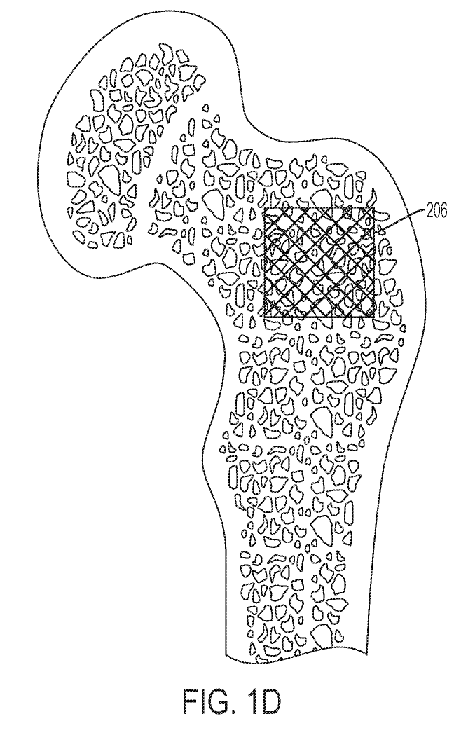

Referring now to the figures, FIGS. 1A and 1B show a woven retention device 100 for interfacing with a bone surface 104, according to an example of an embodiment. The retention device 100, as shown, may have a general configuration or construction in the form of a hollow tubular shape shown as a sleeve body 106 including a plurality of interwoven filaments that may form a substantially tubular lattice. The general configuration of the hollow tubular shape can be selected to accommodate a typical shape of a pilot hole in bone, for example. Various configurations of the sleeve body 106 can be contemplated in accordance with the principles of the invention.

The lattice may include a plurality of protuberances distributed on an interior surface 110 and an exterior surface 108 of the lattice at a predetermined spatial relationship. Each of the plurality of protuberances may be formed by an intersection of filaments. More particularly, each of the plurality of protuberances may be formed by an intersection point of two or more of the plurality of interwoven filaments. The intersection can be referred to as a location and/or point. Additionally, the interwoven filaments may outline apertures that allow for bone ingrowth. The woven retention device can also have a proximal end 114 that is proximal to the sleeve body 106 and that is configured to receive at least a portion of a fastener 102 such that the sleeve body 106 may surround at least a portion of the fastener 102 when inserted therein. The woven retention device 100 can also have a distal end 116 that is distal to the sleeve body 106. In some embodiments, the distal end 116 is formed to ease insertion of the woven retention device 100 into the bone hole 101. For example, the distal end 116 in FIG. 1A is tapered. The lattice can be a tubular lattice.

Embodiments of the woven retention device include a woven retention device to impede biofilm formation. The woven retention system 100 can include a sleeve body 106 comprising a plurality of interwoven filaments that form a substantially tubular lattice with a plurality of protuberances distributed on an interior surface and an exterior surface of the tubular lattice at a predetermined spatial relationship. The woven retention system 100 can include a proximal end that is proximal to the sleeve body and that is configured to receive a fastener; and a distal end that is distal to the sleeve body on an opposing side as the proximal end.

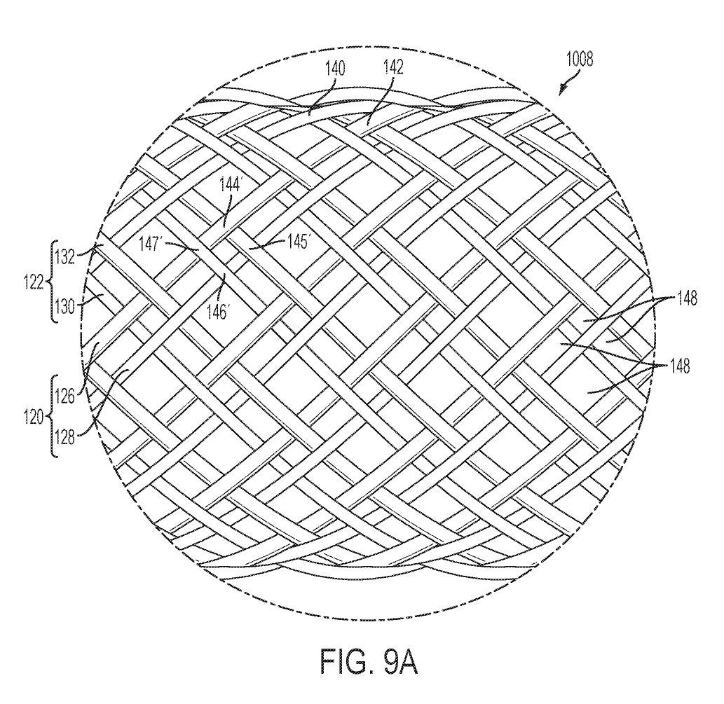

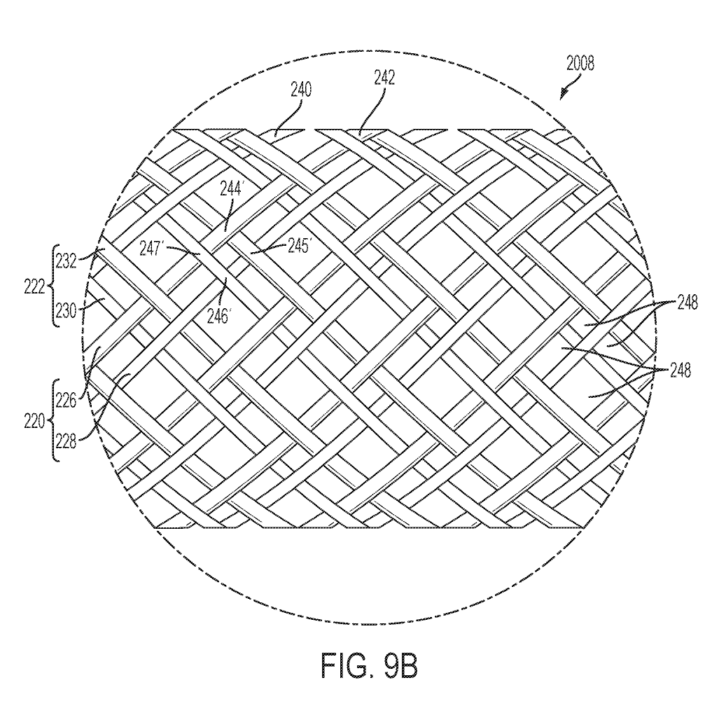

As can be seen in FIG. 9A and as will be described in greater detail, the woven retention device 1008 can be configured such that the intersecting sets of filaments 122, 124 form a plurality of differently shaped and differently sized apertures 148. In one embodiment, as shown in FIG. 9A, the first inner 130 and outer filaments 132 of one set of first filaments 122 can be grouped closer to each other than the other sets 120 of first filaments. Likewise, the second inner 126 and outer filaments 128 of one set 120 of second filaments can be grouped closer to each other than the other sets of second filaments. When the two sets of filaments intersect, as shown in FIG. 9A, the area which is outlined by the first and second plurality of sets of filaments is a plurality of differently shaped and differently sized apertures 148.

The area of the apertures can change dynamically by the interwoven filaments translating with respect to each other without substantial stretching of the interwoven filaments. When the woven retention device is in a constricted, the aperture areas can change by a function of a braid of the filaments.

The area of the aperture can be in a number of various shapes. For example, as shown in FIG. 9A, the apertures 148 can be in a substantially rectangular or square shape. However, other shapes such as circles and ovals are contemplated. The rectangles or squares can be of varying sizes. The size of the shape of the aperture can be measured by a height and/or width for a square, triangle or diamond shape, a long and/or short axis for a rectangle, a diameter for a circle, and a major and/or minor axis for an oval.

By having differently shaped and sized apertures, a more conducive environment for non-uniform bony surface can allow for ingrowth of bone to occur. Additionally, improved interdigitation with the bony structure can be achieved with a combination of the apertures and protuberances.

The spatial relationship of the protuberances of the woven retention device can affect the formation of biofilms on the fastener and/or woven retention device. For example, micro-organisms may attach to the fastener and/or the woven retention device. After attachment, the micro-organisms can mature and clog the apertures of the woven retention device. For example, the apertures of the woven retention device allow for porosity that enable bone ingrowth to occur, which facilitates healing. In embodiments of this invention, porosity and pore sizes of the woven retention device are associated with these and other biological responses. For example, very small pore sizes or apertures make the formation of biofilms easier. One of the first stages of development can include 1) initial attachment of the microorganisms, and 2) irreversible attachment (which can lead to buildup). Embodiments of the invention here relate to both phases of preventing initial attachment by the filaments being thin enough and of a material to resist attachment and also having the pore sizes be large enough to prevent irreversible attachment. Thus, larger pore sizes can prevent biofilm attachment. Further, pore sizes can be affected by the degree of the weave intersections. At a 45 degree braid angle, a maximum pore size relationship can be achieved. Thus, even if a biofilm attaches to the filaments, by having large pore sizes, spreading or maturing of the biofilm can be prevented or slowed. Also, other biological responses besides biofilm formation, such as fibrosis, relate to the porosity of the woven retention device.

Further there may be some materials that prevent the cellular response of biofilms from building up and maturing. In some embodiments, the woven retention device 100 can include an orthopedic biomaterial that impedes or prevents biofilm attachment or maturation and/or that stimulates bone growth. Biomaterials in orthopedics can be made of biocompatible, biofunctional, non-toxic, machinable, moldable, extrudable, having tensile strength, yield strength, elastic modulus, corrosion and fatigue resistance, surface finish, creep, hardness. See Patel and Gohil, “A Review on Biomaterials: Scope, Applications & Human Anatomy Significance,” International Journal of Emerging Technology and Advanced Engineering 2(4): pp. 91-93 (April 2012) (Patel), the content of which is hereby incorporated herein by reference in its entirety. For example, the interwoven filaments can include biomaterials and the biomaterials can be manufactured in fiber format. The woven or non-woven structures described above can be made of non-resorbable or bioabsorbable polymers, metals, biological products or ceramics. Bio resorbable polymer material can be used. For example, the sleeve material can be bioabsorbable and dissolve for complete healing, reduced risk of particulate debris, and have no removal complications as a result. The bioabsorbable polymer can include at least one of thermoplastic aliphatic polyester (PLA), polyglycolide (PGA), polylactide (PLLA) and resorbable polyamides. Alternatively, the sleeve material does not degrade but stays as a structural support of the bone. A non-resorbable polymer material can be biologically suited for use in bone, such as PET (polyehthylene terephthalate), ultra high molecular weight polyethylene, polyether etherketone (PEEK), polyether ketoneketone (PEKK), polypropylene, polyamides, PTFE, calcium phosphate and variations of sutures.

A hydrophilic biomaterial such as a metal can be hydrophilic and attract bone. In one embodiment, metals can also be used such as titanium, tantalum, nickel titanium (nitinol), platinum, cobalt chrome/cobalt chromium, or a blend of all the listed metals. For example, the metals can include at least one of nickel-titanium (Ni—Ti) or nitinol, stainless steel, platinum, titanium, cobalt chrome, cobalt chromium, or any combination thereof. In an embodiment, the metal material can be roughened to create a roughness characteristic that attracts bone, or encourage bone to grow to it or group to it. In an embodiment, the biomaterial such as a metal can have a radioactive property such that the biomaterial can be detected using electromagnetic radiation, such as X-rays. In one embodiment, the woven retention device can be made of fibers of a bone-promoting biomaterial in combination with fibers of a material that does not promote bone growth. For example, the woven retention device can be made of fibers of titanium, which promotes bone growth, as well as PEEK, which promotes bone growth less. Additionally, fibers of PEKK, which can promote bone growth, can be used in combination with titanium and PEEK. In one embodiment, the filaments can include porous fibers.

In an embodiment, the woven retention device can be constructed with an interior surface having a tap of the metallic biomaterial that follows the path of a fastener such as a screw. In such a configuration, the woven retention device is self-tapped to receive an insert, and as the screw follows the path, the woven retention device is configured to expand. In one embodiment, the self-tapping can be produced through the weaving pattern of the fibers or through a mechanical inscribing process that machines thread that matches to the material into which the woven retention device is being inserted. For example, one metal fiber can be included among all other plastic fibers and based on the pitch of the screw, the metal fiber can be designed to follow the tap of the screw.

Biological materials or biologics, such as silk, collagen, and cat gut suture can be used. See Park and Lakes, “Biomaterials: An Introduction,” 1992, Chapter 4 (Park), the content of which is hereby incorporated by reference herein in its entirety. The biological products can include at least one of silk and collagen. Thus, the sleeve can be made of sheet fabric materials such as Silk or Felt that is not woven, but could be created by using collagen. An interior surface could be configured to interface with different structures besides a screw (clamp, smooth, roughened) to provide a strong connection as long as there are many points of contact to provide sufficient sheer strength and a monolithic structure (that is, if one point fails, whole structure does not fail).

In some embodiments, ceramic materials can be used (or bioceramics), which are inert, strong in compression, and biocompatible, such as aluminum oxide, calcium phosphate (hydroxyapatite). See Patel, page 96. For example, the sleeve material can be made of bioactive glass ceramics. See Park chapter 3. The sleeve materials can have bone regenerative qualities (bony apposition).

In some embodiments, the woven retention device 100 can include sleeve materials applied to the woven retention device 100. For example, structural integrity of the woven retention device can be a non-resorbable material, for example PET or PEEK fiber. And the woven retention device is then interlaced with a biologic fiber, such as collagen, to attract or stimulate the bone. Thus, in an embodiment, biological fibers need not provide structural or fixation support, but instead locally stimulate bone formation. Alternatively, the woven retention device can include can include all synthetic materials as non-resorbable materials and instead of interlacing biological fibers, the woven retention device can be coated with an osteostimulative agent.

Instead of a flat fiber, a rougher or more pillowy surface is also possible. In an embodiment with a monofilament/multifilament, a textured fiber instead of a flat multifilament could absorb or wick up more biological agent. In one embodiment, the woven retention device can be biofriendly and have a wicking characteristic to absorb plasma-rich platelets. The sleeve materials can have antibiotic or anti-microbial properties to reduce infections and enhance effectiveness. The woven retention device can be made of a metal that slowly releases ions over time that has anti-microbial properties. Some materials alone have antimicrobial properties. Naturally occurring Silver, for example, can be used as an anti-microbial agent. Thus, impregnating PEEK with Silver can provide an anti-microbial property. Other materials, including metals and polymers, can have anti-microbial properties in combination with other materials. The sleeve material can have drug eluting properties to stimulate bone growth and improve recovery time. And it can be provided at the local level instead of at the systemic level. The sleeve material can also be bioconductive meaning that an allograph fixation sleeve can be made using allographic tissue to create a bone based fixation sleeve in combination with long fiber bone tissue processed by Osteotech, now owned by Medtronic. The allographic tissue can be made out of different material (human based material).

The woven retention device 100 can be inserted into a hole in a bone and interact with both the bone and a screw. While the woven retention device 100 can achieve an interference fit functionality by providing additional interference in between the fastener and the bone, in some embodiments, the woven retention device can instead of and/or in addition to function as a woven retention device in accordance with the configurations, functions and advantages that are discussed herein. For example, the woven retention device can have a dual interface between a radial screw surface on one side and multiple points of contact on a bone surface on the other side. The dual interfaces on the retention device are configured to be adapted to the bony structure on the outside and the screw on the inside, as described herein in accordance with the principles of the invention. The woven retention device can be particularly beneficial for osteoporotic or weakened bone that has more space gaps than normal bone to allow additional points of contact for the interface to contact.

FIG. 1A shows the woven retention device 100 in an exploded state with the fastener 102 outside of the retention device, and both the fastener 102 and the retention device 100 are outside of the bone hole. FIG. 1B shows the fastener 102 inside the woven retention device 100, which is inside the bone. FIGS. 1A and 1B also illustrate an example of a porous interior structure of the bone. However, embodiments of the invention are not limited to being used with the exact porous structure shown, as the structure and porosity of bone can vary. In addition, although the bone illustrated in FIGS. 1A and 1B resembles a human femur, embodiments of the invention are not limited to a particular bone. An advantage of some embodiments of the invention is that a woven retention device can be provided for use in a variety of bones and bones exhibiting varying levels of porosity.

Thus, a woven retention device 100 for interfacing with a bone surface can include a sleeve body 106 comprising a plurality of filaments forming a substantially tubular lattice with a plurality of protuberances distributed on an interior surface and an exterior surface of the tubular lattice at a predetermined spatial relationship. The sleeve body 106 can be configured to surround at least a portion of a fastener 102. Each of the plurality of protuberances can be formed by an intersection point of two or more of the plurality of filaments that outline a plurality of apertures. The sleeve body 106 can include an orthopedic biomaterial

The woven retention device 100 can include a proximal end 114 that is proximal to the sleeve body and that is configured to receive at least a portion of the fastener 102. The woven retention device 100 can include a distal end 116 that is distal to the sleeve body. In a first state, the sleeve body 106 has a plurality of combinations of filament cross-section geometries at the intersection points, the plurality of combinations of filament cross-section geometries forming a plurality of protuberance thicknesses, a thickness of each protuberance being measured in a radial direction of the sleeve body. In a second state when a fastener is inserted into the tubular lattice, pressure from the fastener 102 can be transmitted to the tubular lattice such that the spatial relationship of the protuberances changes according to a function of bone density and according to a function of an interfacing surface shape of the fastener.

The woven retention device 100 can thus be configured to impede biofilm formation. The biomaterial of the woven retention device 100 can be made of a material that impedes biofilm formation. The sleeve body can have a structure that impedes biofilm formation.

The sleeve body can be configured to receive a portion of the soft tissue and the sleeve body is configured to impede biofilm formation surrounding the soft tissue. The sleeve body comprises a coating on the plurality of filaments, wherein the coating comprises an orthopedic biomaterial. The plurality of filaments can comprise the orthopedic biomaterial.

The woven retention device can include an orthopedic biomaterial of one of the following: PLA, PGA, PLLA, PET, PEEK, PEKK, polypropylene, polyamides, PTFE, calcium phosphate, platinum, cobalt chrome, nitinol, stainless steel, titanium, PEEK, silk and collagen, bioceramics, aluminum oxide, calcium phosphate, hydroxyapatite, glass ceramics, or any combination thereof.

Referring now to the figures, FIGS. 1C and 1D show a woven patch 200 for interfacing with a bone surface 204, according to an example of an embodiment. The patch 200, as shown, may have a general configuration or construction in the form of a patch shown as a sleeve body 206 including a plurality of interwoven filaments that may form a lattice. The general configuration of the lattice can be flat and adapted to accommodate a typical shape of a bone, for example. The woven patch can have a degree of returnability or flexibility to adapt to various bone structures. Additionally, various configurations of the sleeve body 206 can be contemplated in accordance with the principles of the invention.

The lattice may include a plurality of protuberances distributed on a first surface, or an interior surface 210, and a second surface, or an exterior surface 208, of the lattice at a predetermined spatial relationship. Each of the plurality of protuberances may be formed by an intersection of filaments. More particularly, each of the plurality of protuberances may be formed by an intersection point of two or more of the plurality of interwoven filaments. The intersection can be referred to as a location and/or point. Additionally, the interwoven filaments may outline interstices that allow for bone ingrowth. The woven patch can also have a proximal end 214 that is proximal to the sleeve body 206 and that is configured to be applied to at least a portion of a fastener 202. The woven patch 200 can also have a distal end 216 that is distal to the sleeve body 206. In some embodiments, the distal end 216 is formed to ease insertion of the woven patch 200. For example, the distal end 216 in FIG. 1C is tapered. The lattice can be a tubular lattice.

The woven patch 200 can be applied to a bone and interact with both the bone and a screw. While the woven patch 200 can achieve an interference fit functionality by providing additional interference in between a fastener and the bone, in some embodiments, the woven patch can instead of and/or in addition to function as a woven patch in accordance with the configurations, functions and advantages that are discussed herein. For example, the woven patch can have a dual interface between a radial screw surface on one side and multiple points of contact on a bone surface on the other side. The dual interfaces on the patch are configured to be adapted to the bony structure on the outside and the screw on the inside, as described herein in accordance with the principles of the invention. The woven patch can be particularly beneficial for osteoporotic or weakened bone that has more space gaps than normal bone to allow additional points of contact for the interface to contact.

FIG. 1C shows the woven patch 200 in an exploded state outside of the bone placement. FIG. 1D shows the woven patch 200 applied to the bone. FIGS. 1C and 1D also illustrate an example of a porous interior structure of the bone. However, embodiments of the invention are not limited to being used with the exact porous structure shown, as the structure and porosity of bone can vary. In addition, although the bone illustrated in FIGS. 1C and 1D resembles a human femur, embodiments of the invention are not limited to a particular bone. An advantage of some embodiments of the invention is that a woven patch can be provided for use in a variety of bones and bones exhibiting varying levels of porosity. Further, although the woven patch of FIGS. 1C and 1D cover a small portion of the bone surface, the woven patch 200 can also be applied around a region of a bone or to an entire surface of the bone.

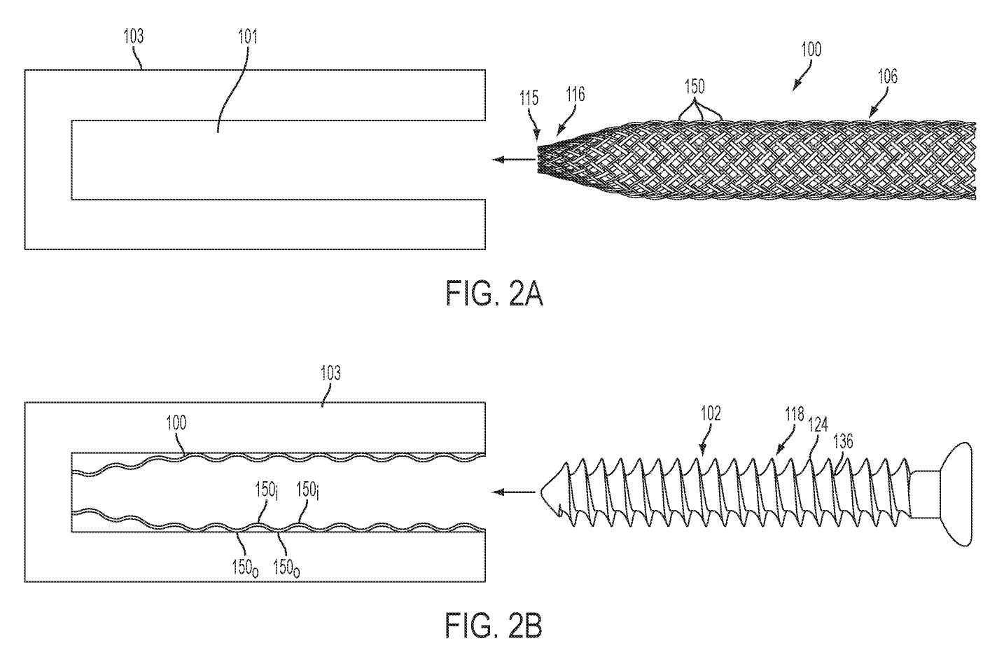

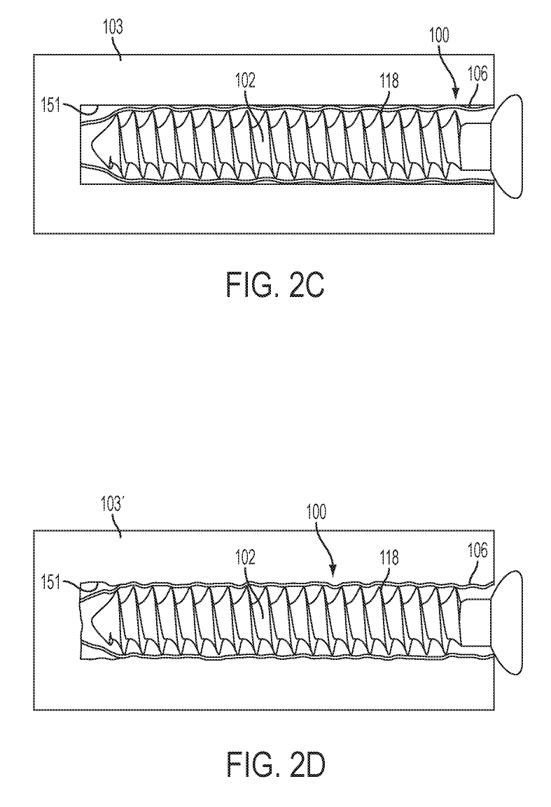

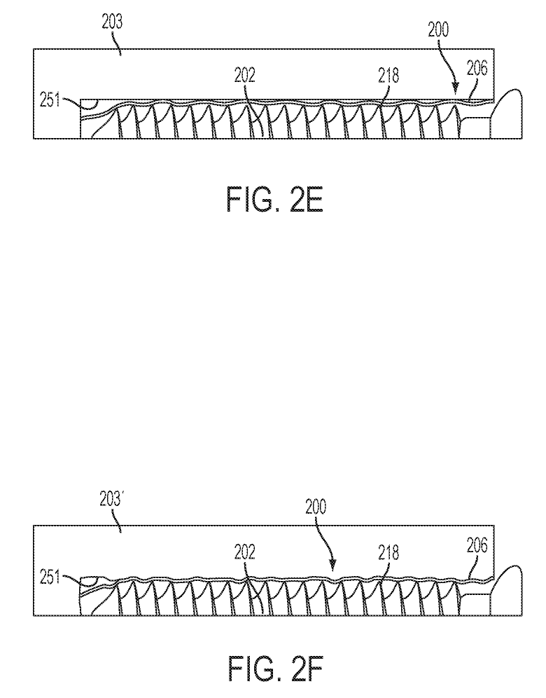

FIG. 2A shows a sleeve body 106 to be inserted into a bone hole 101 in a bone 103. According to this embodiment, the distal end 116 tapers to a distal tip 115 that has a smaller diameter than the sleeve body 106. The tapering at the distal tip 115 can ease insertion of the woven retention device 100 into the bone hole 101. For example, in some embodiments, the diameter of the sleeve body 106 may be equal to or larger than a diameter of the bone hole 101, and the tapering at the distal tip 115 can allow the distal end 116 to find its way into the bone hole 101. For example, after the distal end 116 is at least partially inserted into the bone hole 101, a remainder of the woven retention device 100 can more easily be inserted into the bone hole 101, and, in a case where the diameter of the sleeve body 106 is larger than the diameter of the bone hole 101, the woven retention device 100 can compress radially as the sleeve body 106 is inserted into the bone hole 101. In addition, as discussed further below, the tapering of the distal end 116 and smaller diameter of the distal tip 115 can provide a surface on the interior of the woven retention device 100 for pushing against with a push rod to insert the woven retention device 100 into the bone hole 101, according to some embodiments. The woven retention device 100 may be in a first, relaxed state at the position shown in FIG. 2A. During or after insertion into the bone hole 101, however, the woven retention device 100 may also assume a radially contracted or radially expanded state.

The plurality of interwoven filaments, according to an embodiment of the woven retention device 100, are visible in FIG. 2A. As discussed in detail further below, these filaments may include one or more varieties of filament shapes and sizes such that the sleeve body 106 can have a plurality of combinations of filament cross-section geometries at the intersection of the filaments, which can also be referred to as intersection points of the filaments. Because each intersection of the filaments may form a protuberance 150, the plurality of combinations of filament cross-section geometries may form a plurality of protuberance thicknesses, each thickness being measured in a radial direction of the sleeve body 106. For example, a cross-section geometry can include a shape of the cross-section and/or a size of the cross-section. The combination of the filament cross-section geometries can include the cross-section geometries of both filaments at the intersection.

FIG. 2B shows a simplified schematic cross-section of the bone hole 101 and the woven retention device 100 inserted therein. For example, the undulating lines representing the sides of the woven retention device 100 in FIG. 2B may represent the plurality of protuberances 150o, 150i on the exterior and interior, respectively, of the woven retention device 100 by the series of peaks formed on the respective walls of the woven retention device 100. In the implanted state the woven retention device 100 is adapted to receive the fastener 102. In one embodiment, the fastener 102 can be a screw having a winding protrusion 118, such as a thread of a screw. The woven retention device 100 can be configured such that when the protrusion 118 applies pressure to a protuberance on the interior surface, the pressure is transmitted to protrusions on the exterior surface extending around the protrusion on the interior surface and exerting pressure on bone material.

Embodiments of the invention are not limited to being used with a screw-type fastener. In some embodiments, the fastener may be a nail, rod, prosthetic, or other device for implanting at least partially in a bone. Additionally, in some embodiments a biological material or structure, such as a ligament, may be inserted into the woven retention device.

FIGS. 2C and 2D show that in a second state, when surrounding at least a portion of the fastener 102, the sleeve body 106 is configured to engage the bone 103 surrounding the bone hole 101, and may distribute pressure from the fastener 102 to multiple points of contact on the exterior surface of the woven retention device 100 such that the spatial relationship of the plurality of protuberances may change. The spatial relationship of the plurality of protuberances may change as a function of bone density of the bone surface 104. For example, FIG. 2C shows a bone 103 that is more dense than the bone 103′ of FIG. 2D. Thus, when the fastener 102 applies pressure on the woven retention device 100, the woven retention device 100 is displaced more prominently in the less dense surface of FIG. 2D than the denser bone surface of FIG. 2C. This displacement of the woven retention device 100 corresponds to a change in the spatial relationship of the plurality of protuberances (the protuberances themselves are not shown in the simplified schematic view of FIGS. 2C and 2D) on the exterior surface, which can allow for greater interdigitation of the woven retention device 100 with the bone surface. In one embodiment, the force from the protuberances on the exterior surface changes the shape of the bone. It is noted that the illustration of the bones 103 and 103′ in FIGS. 2C and 2D are simplified schematic representations. In practice, the surfaces of the bones 103 and 103′ that are engaged by the woven retention device 100 may be irregular, including a series of voids and projections, for example. Accordingly, the variation in displacement of the sides of the woven retention device 100 when the fastener 102 is inserted can accomplish improved engagement between the woven retention device 100 and the bone 103 (and correspondingly provide the fastener 102 with greater purchase in the bone).

The spatial relationship of the plurality of protuberances can also change as a function of loading and/or the fastener. The spatial relationship of the plurality of protuberances can change as a function of an interfacing surface shape of the fastener 102. As shown in FIG. 2C, the fastener 102 can be a screw. In one embodiment, the screw can be a cancellous screw. In another embodiment, the screw can be a cortical screw. The screw can have crests 124 that are the most outwardly protruding portions of the thread of the screw and can have valleys 136, which are the innermost portions of the screws. The screw can have various levels of coarseness of the threads, representing larger pitch (fewer threads per axial distance). In one embodiment, where the screw has a larger pitch, for instance in a larger size of screw, the retention device when interfacing with the screw can change to accommodate the coarse threads. For example, the retention device can adapt to follow the crests 124 and the valleys 136 to create a general wave pattern. On the other hand, in the case of a smaller diameter screw, or a finer thread with smaller pitch, the retention device can deform or bend over the peaks of the threads less. Thus, in one embodiment, the absolute value of pullout resistance can be greater with a larger screw but the delta between the differential can be smaller with the larger diameter screw because of additional interwinding of the intermediary point of contact. That is, in one embodiment, the protuberances on the exterior surface do not interface as much with the bone because of some of the protuberances folding inward because of the coarseness of the thread. Whereas on the small diameter screw, the woven retention device can move more uniformly, which can allow for greater interdigitation. Thus, because there can be less chance for those interdigitation points to reach into the valleys of the threads, there is more interaction with the bony surface.

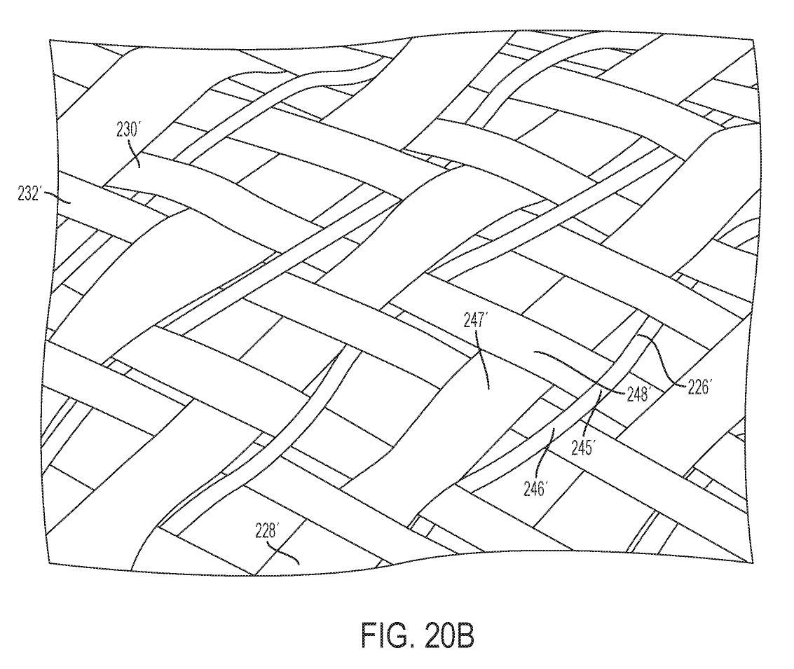

The spatial relationship of the plurality of protuberances can also change as a function of an interfacing surface shape based on the length of the surface. For example, the surface of the fastener 102 can also be various lengths. As seen from FIGS. 20-22, even though the change in pullout resistance can be greater with large screws than small screws in total pullout resistance, the small screw can have greater pullout resistance as a measure of percent change. One factor that affects the small screw having a greater pullout resistance in percent change is that more interaction with the woven retention device 100 can be possible with a smaller fastener as a percentage of the fastener's percentage of coverage. This can result in a larger differential in pull out resistance in the smaller sizes than there is in the larger sizes because of the increased interaction. In one embodiment, the mechanical properties of the woven retention device can compensate for differences in the fastener surface. For example, to increase bone surface interaction with a fastener 102 that has a coarse thread, a woven retention device with a greater level of stability can be used to prevent the filaments from retreating too far into the valleys 136 and instead interacting with the bone surface.

In some embodiments, the woven retention device 100 may be specifically configured for a bone of a particular density or range of densities. For example, the structural configuration, material properties, or other aspects of the woven retention device may be adjusted to provide desired engagement with the bone surface of a particular density or range of densities. However, in some embodiments, a particular woven retention device may be suitable for use in bones of varying densities.

FIG. 2E shows a patch 206 interfaced with a bone 203. The patch 206 can be woven or non-woven. Because each intersection of the filaments may form a protuberance 250, the plurality of combinations of filament cross-section geometries may form a plurality of protuberance thicknesses, each thickness being measured in a thickness of the sleeve body 206. For example, a cross-section geometry can include a shape of the cross-section and/or a size of the cross-section. The combination of the filament cross-section geometries can include the cross-section geometries of both filaments at the intersection. The woven patch 200 can be configured such that when the protrusion 218 applies pressure to a protuberance on the interior surface, the pressure is transmitted to protrusions on the exterior surface extending around the protrusion on the interior surface and exerting pressure on bone material.

Before application or insertion onto or into a bone surface, the woven patch 200 may be in a first, relaxed state at the position. During or after insertion into or after application onto the bone 203, however, the woven patch 200 may also assume a contracted or expanded state. The plurality of interwoven filaments, according to an embodiment of the woven patch 200, are visible in FIG. 2E. As discussed in detail further below, these filaments may include one or more varieties of filament shapes and sizes such that the sleeve body 206 can have a plurality of combinations of filament cross-section geometries at the intersection of the filaments, which can also be referred to as intersection points of the filaments. Because each intersection of the filaments may form a protuberance 250, the plurality of combinations of filament cross-section geometries may form a plurality of protuberance thicknesses, each thickness being measured in a thickness of the woven patch 200. For example, a cross-section geometry can include a shape of the cross-section and/or a size of the cross-section. The combination of the filament cross-section geometries can include the cross-section geometries of both filaments at the intersection.

FIG. 2F shows a simplified schematic cross-section of the bone 203 and the woven patch 200 inserted therein. For example, the undulating lines representing the sides of the woven patch 200 in FIG. 2E may represent the plurality of protuberances 250o, 250i on the exterior and interior, respectively, of the woven patch 200 by the series of peaks formed on the respective walls of the woven patch 200. In the implanted state the woven patch 200 is adapted to receive the fastener 202. In one embodiment, the fastener 202 can be a screw having a winding protrusion 218, such as a thread of a screw. The woven patch 200 can be configured such that when the protrusion 218 applies pressure to a protuberance on the first or interior surface, the pressure is transmitted to protrusions on the second or exterior surface extending around the protrusion on the interior surface and exerting pressure on bone material.

Embodiments of the invention are not limited to being used with a screw-type fastener. In some embodiments, the fastener may be a nail, dowel, rod, prosthetic, or other device for implanting at least partially in a bone. Additionally, in some embodiments a biological material or structure, such as a ligament, may be inserted into the woven patch.

FIGS. 2E and 2F show that in a second state, when surrounding at least a portion of the fastener 202, the sleeve body 206 is configured to engage the bone 203, and may distribute pressure from the fastener 202 to multiple points of contact on the exterior surface of the woven patch 200 such that the spatial relationship of the plurality of protuberances may change. The spatial relationship of the plurality of protuberances may change as a function of bone density of the bone surface 204. For example, FIG. 2E shows a bone 203 that is denser than the bone 203′ of FIG. 2F. Thus, when the fastener 202 applies pressure on the woven patch 200, the woven patch 200 is displaced more prominently in the less dense surface of FIG. 2F than the denser bone surface of FIG. 2E. This displacement of the woven patch 200 corresponds to a change in the spatial relationship of the plurality of protuberances (the protuberances themselves are not shown in the simplified schematic view of FIGS. 2E and 2F) on the exterior surface, which can allow for greater interdigitation of the woven patch 200 with the bone surface. In one embodiment, the force from the protuberances on the exterior surface changes the shape of the bone. It is noted that the illustration of the bones 203 and 203′ in FIGS. 2E and 2F are simplified schematic representations. In practice, the surfaces of the bones 203 and 203′ that are engaged by the woven patch 200 may be irregular, including a series of voids and projections, for example. Accordingly, the variation in displacement of the sides of the woven patch 200 when the fastener 202 is inserted can accomplish improved engagement between the woven patch 200 and the bone 203 (and correspondingly provide the fastener 202 with greater purchase in the bone).

The spatial relationship of the plurality of protuberances can also change as a function of loading and/or the fastener. The spatial relationship of the plurality of protuberances can change as a function of an interfacing surface shape of the fastener 202. As shown in FIG. 2E, the fastener 202 can be a screw. In one embodiment, the screw can be a cancellous screw. In another embodiment, the screw can be a cortical screw. The screw can have crests 224 that are the most outwardly protruding portions of the thread of the screw and can have valleys 236, which are the innermost portions of the screws. The screw can have various levels of coarseness of the threads, representing larger pitch (fewer threads per axial distance). In one embodiment, where the screw has a larger pitch, for instance in a larger size of screw, the patch when interfacing with the screw can change to accommodate the coarse threads. For example, the patch can adapt to follow the crests 224 and the valleys 236 to create a general wave pattern. On the other hand, in the case of a smaller diameter screw, or a finer thread with smaller pitch, the patch can deform or bend over the peaks of the threads less. Thus, in one embodiment, the absolute value of pullout resistance can be greater with a larger screw but the delta between the differential can be smaller with the larger diameter screw because of additional interwinding of the intermediary point of contact. That is, in one embodiment, the protuberances on the exterior surface do not interface as much with the bone because of some of the protuberances folding inward because of the coarseness of the thread. Whereas on the small diameter screw, the woven patch can move more uniformly, which can allow for greater interdigitation. Thus, because there can be less chance for those interdigitation points to reach into the valleys of the threads, there is more interaction with the bony surface.

The spatial relationship of the plurality of protuberances can also change as a function of an interfacing surface shape based on the length of the surface. For example, the surface of the fastener 202 can also be various lengths. One factor that affects the small screw having a greater pullout resistance in percent change is that more interaction with the woven patch 200 can be possible with a smaller fastener as a percentage of the fastener's percentage of coverage. This can result in a larger differential in pull out resistance in the smaller sizes than there is in the larger sizes because of the increased interaction. In one embodiment, the mechanical properties of the woven patch can compensate for differences in the fastener surface. For example, to increase bone surface interaction with a fastener 202 that has a coarse thread, a woven patch with a greater level of stability can be used to prevent the filaments from retreating too far into the valleys 236 and instead interacting with the bone surface.

In some embodiments, the woven patch 200 may be specifically configured for a bone of a particular density or range of densities. For example, the structural configuration, material properties, or other aspects of the woven patch may be adjusted to provide desired engagement with the bone surface of a particular density or range of densities. However, in some embodiments, a particular woven patch may be suitable for use in bones of varying densities.

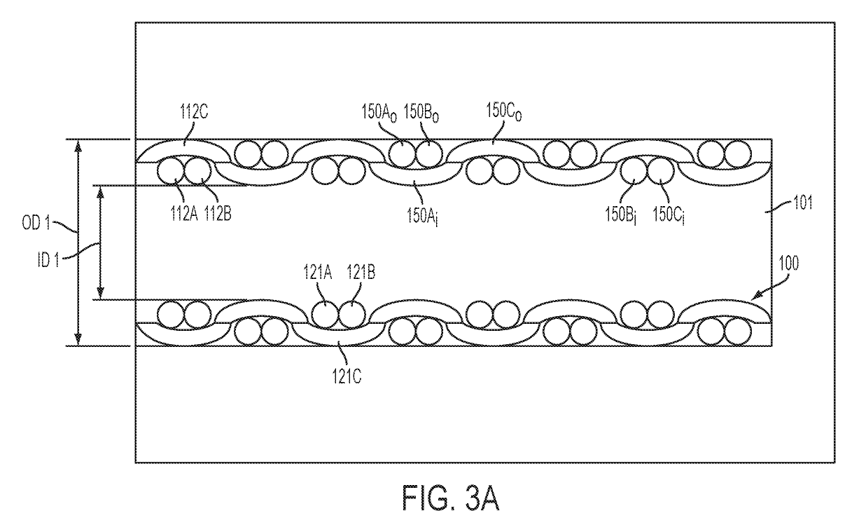

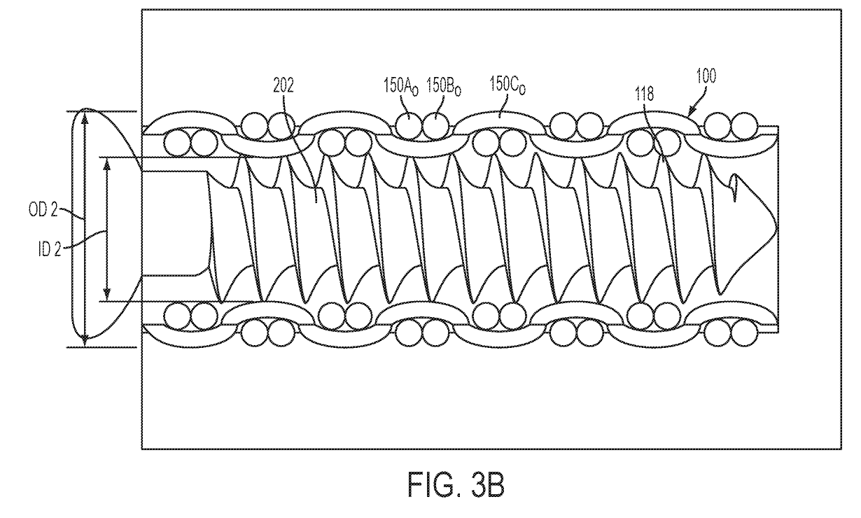

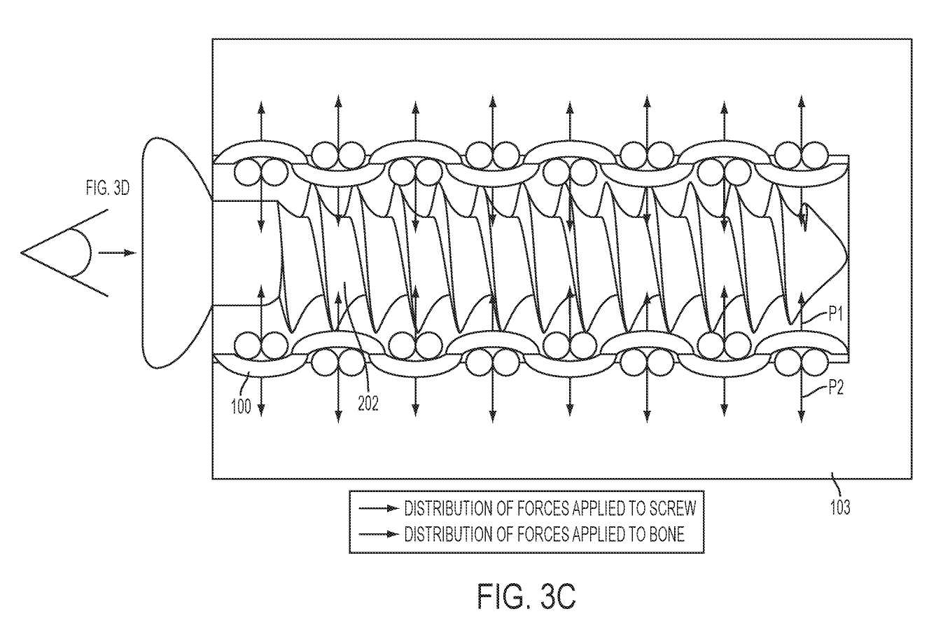

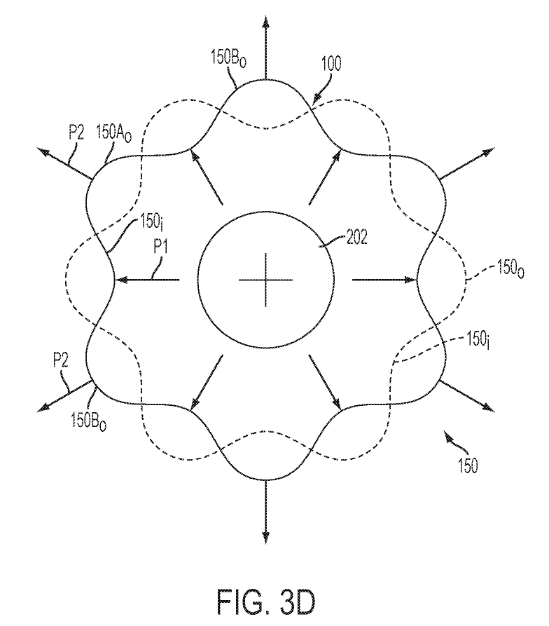

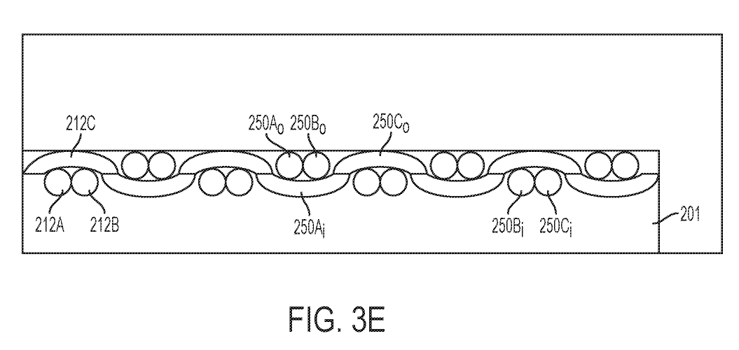

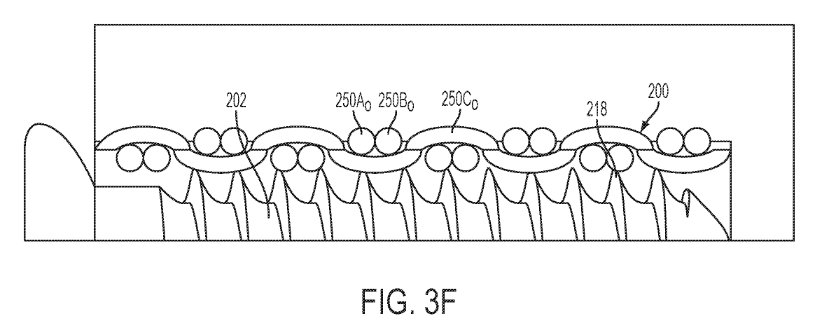

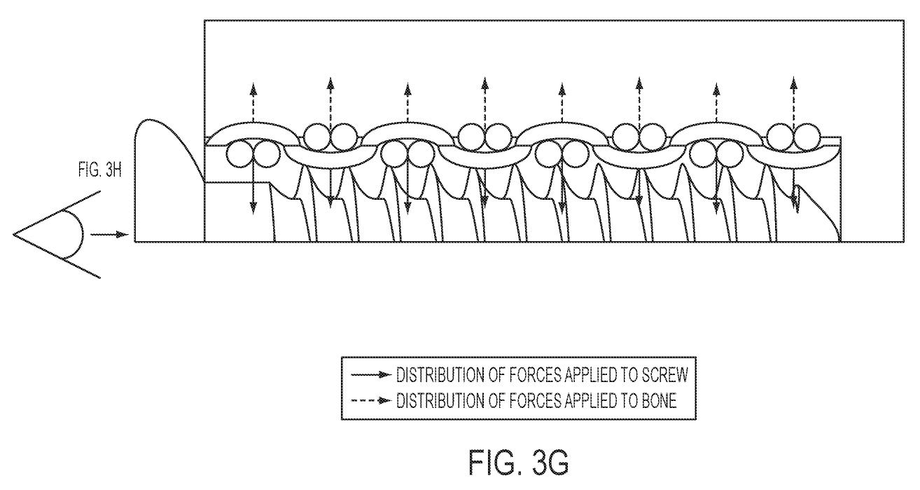

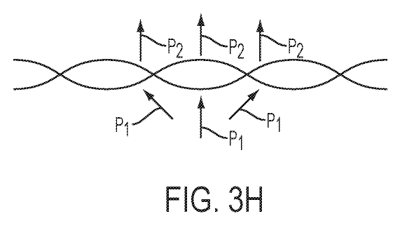

FIG. 3A shows an alternative schematic representation of a cross-section of the woven retention device 100, according to an embodiment. The cross-section of the woven retention device in FIG. 3A reveals an example of the constituent filament cross-section geometries 121A, 121B, 121C that contribute to the protuberances 150Ao, 150Bo, 150Co, 150Ai, 150Bi, 150Ci of the woven retention device 100. At least one filament 112C can be seen weaving over and under adjacent filaments 112A, 112B with substantially circular cross-sections. The woven retention device in FIG. 3A is positioned within the bone hole 101 but without the fastener, resulting in an inner diameter ID1 and an outer diameter OD1 of the woven retention device 100. Outer diameter OD1 can be a distance from an outermost protuberance on the exterior surface of one side to a protuberance on the exterior surface on the opposing side. Further, inner diameter ID1 can be a distance from an innermost protuberance on the interior surface of one side to an innermost protuberance on the interior surface of an opposing side. The relationship between the outer diameter OD1 and the inner diameter ID1 can be based on the thickness or diameter of the filaments.

FIG. 3B shows an alternative fastener 202 that interfaces with the woven retention device 100. As can be seen, FIG. 3B shows that the fastener 202 changes the spatial relationship of the protuberances (e.g., protuberances 150o) from the spatial relationship in FIG. 3A. While outer diameter OD2 can still be a distance from an outermost protuberance on the exterior surface of one side to a protuberance on the exterior surface on the opposing side and inner diameter ID2 can still be a distance from an innermost protuberance on the interior surface of one side to an innermost protuberance on the interior surface of an opposing side, a change in the spatial relationship can result in a larger inner diameter ID2 and outer diameter OD2. The OD2 distance can be a distance larger than outer diameter OD1. Similarly, in one embodiment, the distance ID2 can be larger than the distance ID1. The amount of change of the spatial relationship of the protuberances may change based on the alternative constructions of the fasteners 102 and 202 in FIGS. 2C and 3B, respectively. For example, bone screws are provided in various size and types, which may have different minor diameters, major diameters, thread pitches, pitch diameters, and lengths.

The change in the spatial relationship of the protuberances between FIGS. 3A and 3B can be, in one embodiment, understood as a radial expansion of the woven retention device 100 upon insertion of a fastener therein. This radial expansion can be substantially uniform, as indicated by the uniform displacement of the woven retention device 100 from ID1, OD1 in FIG. 3A to ID2, OD2 in FIG. 3B. As the radial expansion occurs, the spatial relationship between the protuberances on the surface of the woven retention device changes (i.e., the protuberances may spread apart from one another like points on the surface of an inflating balloon). However, according to some embodiments of the present invention, the woven retention device 100 may not expand uniformly when in the bone hole 101. For example, depending on the specific structure of a non-uniform bone surface within the bone hole 101, and depending on the characteristics of the fastener and configuration of the woven retention device, the change in spatial relationship of the protuberances may not be uniform, and may instead include localized changes in the protuberance dispositions. Such localized changes may occur in multiple areas of the woven retention device and may include varying degrees of disposition changes between different areas of the retention device. This capability and/or flexibility provided by some embodiments of the present invention may provide for better bone engagement and fastener retention.

FIG. 3C shows the different radial pressures that can be applied when the fastener 202 is inserted into the woven retention device 100, which is disposed in the bone 103. For example, after the fastener 202 is inserted, the outward radial pressure supplied by the fastener alters the disposition of the woven retention device 100, as discussed above. As a result, pressure P1 is exerted between the retention device 100 and fastener 202. Thus, corresponding pressure P2 is exerted between the retention device 100 and the bone 103 from the pressure transferred by the retention device 100 from the fastener 202. In other words, the woven retention device 100 is a dual-interface. The dual interface includes an inner, fastener-retention device interface and an outer, retention device-bone interface. These two interfaces work in conjunction to provide improved fastener retention and holding power in the bone.

FIG. 3D shows a schematic axial view of the retention device 100 and fastener 202 that are shown in FIG. 3C. For clarity with respect to the pressure forces P1, FIG. 3D shows space between the fastener 202 and an inside of the woven retention device 100, and thus FIG. 3D is not to scale. From the perspective in FIG. 3D, it can be appreciated that the pressures P1 and P2 can radiate in all directions with respect to a center of the woven retention device 100. In addition, according to some embodiments of the invention, a pressure P1 exerted between the fastener 202 and an interior protuberance 150i can be transferred through the woven retention device 100 to multiple exterior protuberances 150Ao and 150Bo to exert pressure P2 at multiple points of contact with the bone.

FIG. 3E shows an alternative schematic representation of a cross-section of the woven patch 200, according to an embodiment. The cross-section of the woven patch in FIG. 3E reveals an example of the constituent filament cross-section geometries 221A, 221B, 221C that contribute to the protuberances 250Ao, 250Bo, 250Co, 250Ai, 250Bi, 250Ci of the woven patch 200. At least one filament 212C can be seen weaving over and under adjacent filaments 212A, 212B with substantially circular cross-sections. The woven patch in FIG. 3E is positioned to interface with bone 203 but without the fastener, resulting in a thickness of the woven patch 200. The relationship between the thickness of the woven patch can be based on the thickness or diameter of the filaments and based on how the interwoven filaments.

FIG. 3F shows an alternative fastener 302 that interfaces with the woven patch 200. As can be seen, FIG. 3F shows that the fastener 302 changes the spatial relationship of the protuberances (e.g., protuberances 250o) from the spatial relationship in FIG. 3E. While the thickness can still be a distance from an outermost protuberance on the exterior surface of one side to a protuberance on the exterior surface on the opposing side, a change in the spatial relationship can result in a larger or smaller thickness. The amount of change of the spatial relationship of the protuberances may change based on the alternative constructions of the fasteners 202 and 302 in FIGS. 2C and 3B, respectively. For example, bone screws are provided in various sizes and types, which may have different minor diameters, major diameters, thread pitches, pitch diameters, and lengths.

The change in the spatial relationship of the protuberances between FIGS. 3E and 3F can be, in one embodiment, understood as an expansion of the woven patch 200 upon insertion of a fastener therein. In one embodiment, the expansion can be radial expansion, which is used only as an example and the woven patch need not be arranged in a radial configuration. In one embodiment, this radial expansion can be substantially uniform, as indicated by the uniform displacement of the woven patch 200 from ID1, OD1 in FIG. 3E to ID2, OD2 in FIG. 3F. As the expansion occurs, the spatial relationship between the protuberances on the surface of the woven patch changes (i.e., the protuberances may spread apart from one another like points on the surface of an inflating balloon). However, according to some embodiments of the present invention, the woven patch 200 may not expand uniformly against a surface of bone 203. For example, depending on the specific structure of a non-uniform bone surface of a surface of the bone 203, and depending on the characteristics of the fastener and configuration of the woven patch, the change in spatial relationship of the protuberances may not be uniform, and may instead include localized changes in the protuberance dispositions. Such localized changes may occur in multiple areas of the woven patch and may include varying degrees of disposition changes between different areas of the patch. This capability and/or flexibility provided by some embodiments of the present invention may provide for better bone engagement and fastener retention.

FIG. 3G shows the different radial pressures that can be applied when the fastener 302 is inserted into or against the woven patch 200, which is disposed in the bone 203. For example, after the fastener 202 is inserted or applied, the outward radial pressure supplied by the fastener alters the disposition of the woven patch 200, as discussed above. As a result, pressure P1 is exerted between the patch 200 and fastener 302. Thus, corresponding pressure P2 is exerted between the patch 200 and the bone 203 from the pressure transferred by the patch 200 from the fastener 302. In other words, the woven patch 200 has a dual-interface. The dual interface includes an inner, fastener-patch interface and an outer, patch-bone interface. These two interfaces work in conjunction to provide improved fastener retention and holding power in the bone.

FIG. 3H shows a schematic view of the patch 200 shown in FIG. 3G. For clarity with respect to the pressure forces P1, FIG. 3H shows space between the fastener 302 and an inside of the woven patch 200, and thus FIG. 3H is not to scale. From the perspective in FIG. 3H, it can be appreciated that the pressures P1 and P2 can radiate in all directions with respect to a center of the woven patch 200. In addition, according to some embodiments of the invention, a pressure P1 exerted between the fastener 302 and an interior protuberance 250i can be transferred through the woven patch 200 to multiple exterior protuberances 250Ao and 250Bo to exert pressure P2 at multiple points of contact with the bone.

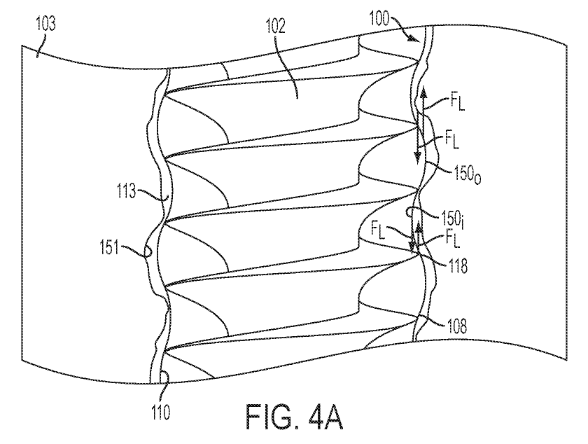

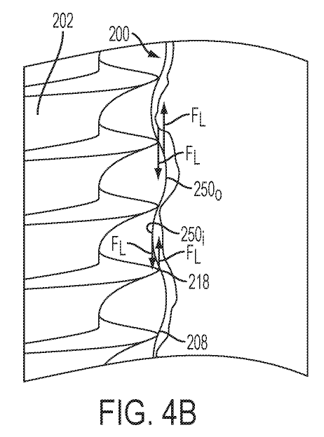

FIG. 4A shows a side view of a fastener 102 inside of a retention device 100 and inside a bone hole 101, according to an embodiment of the present invention. Longitudinal forces FL can act between interdigitated portions of the retention device (e.g., the protuberances 150i, 150o) and the bone surface 151, and between the retention device 100 and the fastener 102. These longitudinal forces can act to prevent pullout of the fastener 102, which can add to the resiliency of the fastener 102 in the longitudinal direction. For example, because of the interaction between the protuberances 150i on the interior surface 110 of the woven retention device 100 and the surface of the fastener (which can optionally be screw ridge 118), there is increased resistance and it is more difficult for the fastener 102 to be pulled out. Similarly, because of the interaction between the protuberance 150o on the exterior surface 108 and the surface 151 of the bone 103, there is increased resistance to pullout the fastener 102. FIG. 4 also shows a gap 113 between the exterior surface 108 of the woven retention device 100 and the bone surface 151. The gap 113 may be smaller or larger depending on the porosity of the bone 103, the configuration of the woven retention device 100, and the characteristics of the fastener 102.

FIG. 4B shows a side view of a fastener 202 inside of a patch 200 and against the bone 203, according to an embodiment of the present invention. Longitudinal forces FL can act between interdigitated portions of the patch (e.g., the protuberances 250i, 250o) and the bone surface 251, and between the patch 200 and the fastener 202. These longitudinal forces can act to prevent pullout of the fastener 202, which can add to the resiliency of the fastener 202 in the longitudinal direction. For example, because of the interaction between the protuberances 250i on the interior surface 210 of the woven patch 200 and the surface of the fastener (which can optionally be screw ridge 218), there is increased resistance and it is more difficult for the fastener 202 to be pulled out. Similarly, because of the interaction between the protuberance 250o on the exterior surface 208 and the surface 251 of the bone 203, there is increased resistance to pullout the fastener 202. FIG. 4B also shows a gap 213 between the exterior surface 208 of the woven patch 200 and the bone surface 251. The gap 213 may be smaller or larger depending on the porosity of the bone 203, the configuration of the woven patch 200, and the characteristics of the fastener 202.

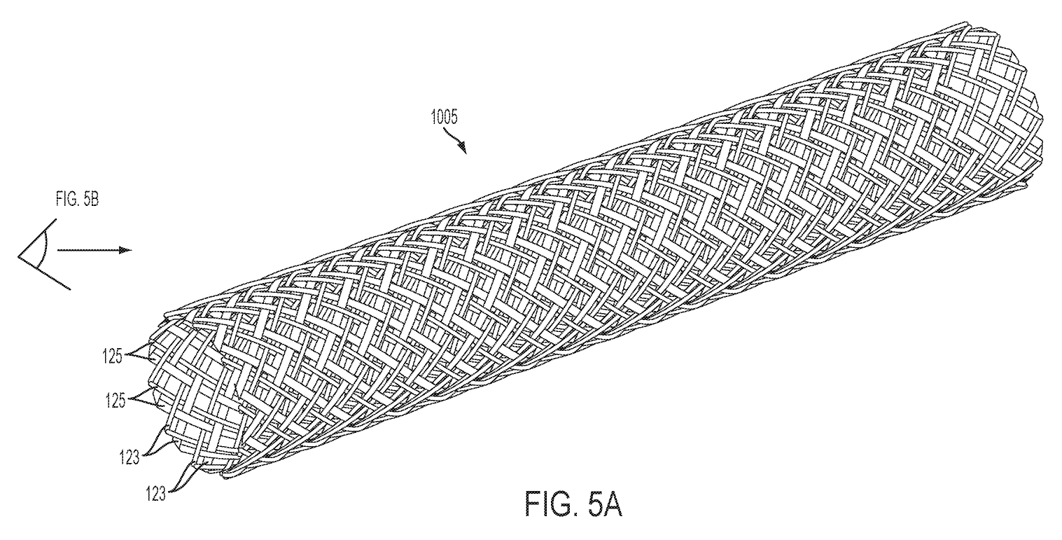

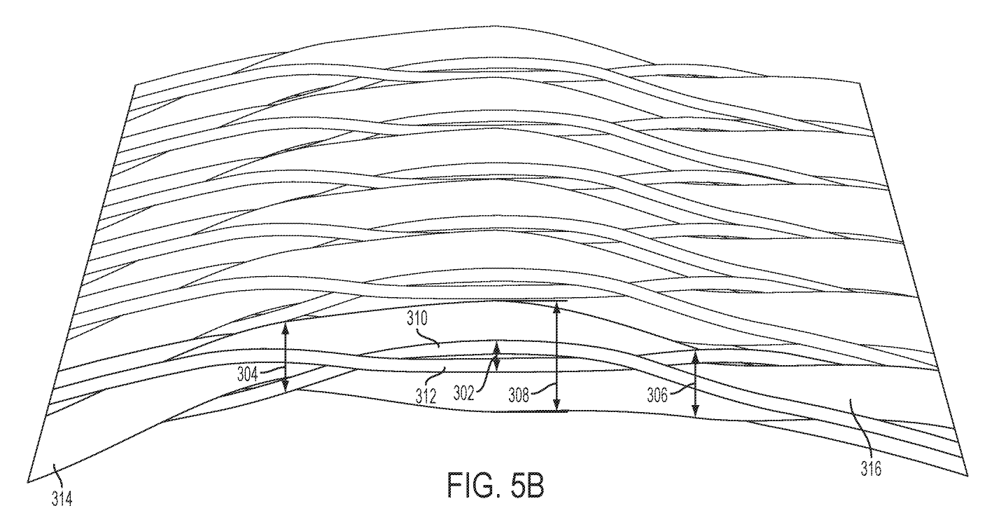

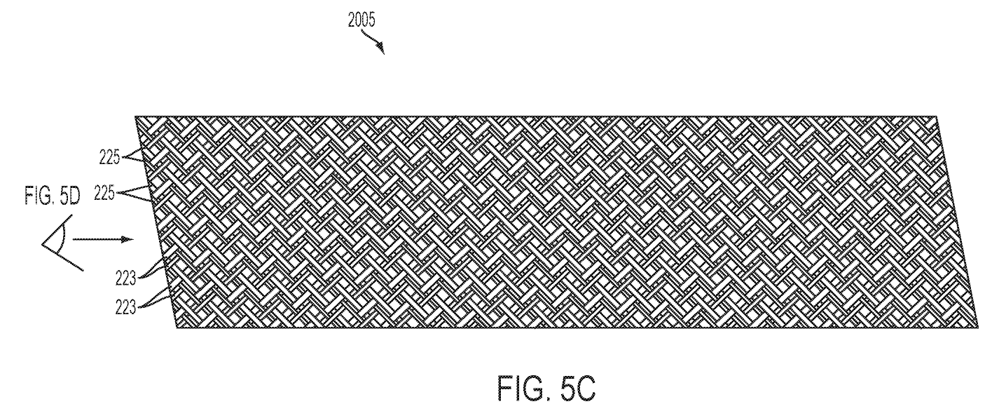

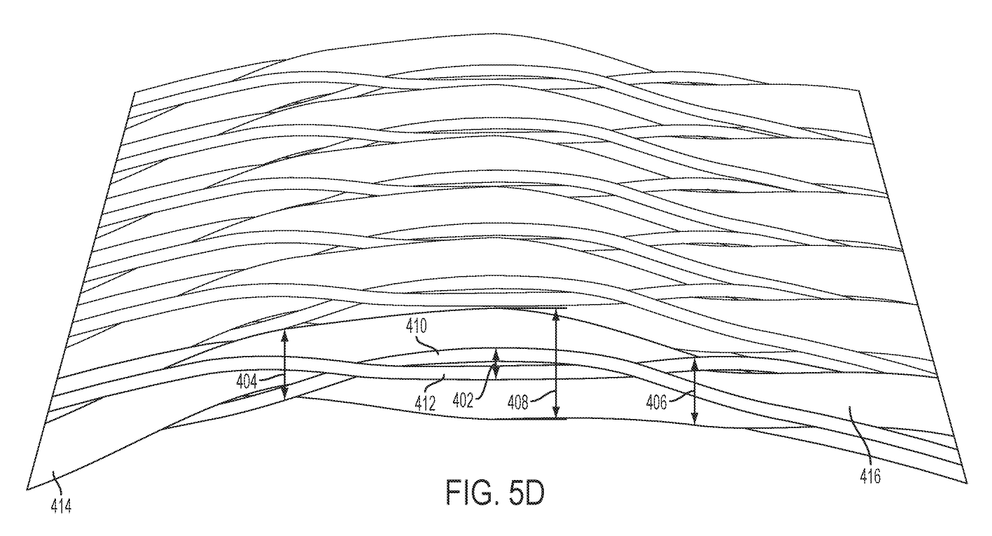

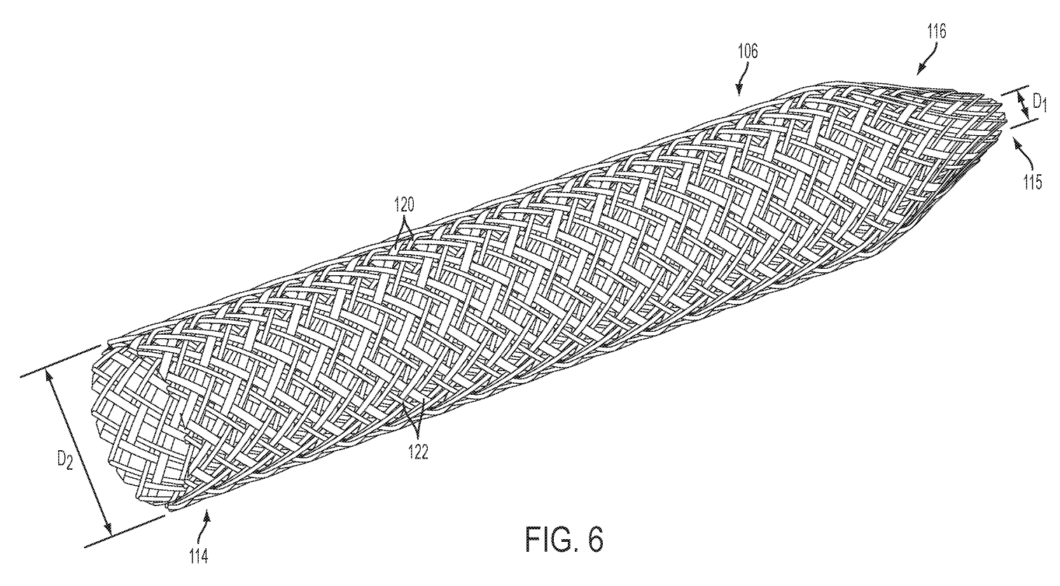

FIG. 5A shows a woven retention device 1005 according to an embodiment of the invention. In this embodiment, the interwoven filaments of the woven retention device 100 can include a first plurality 123 of sets of filaments 120 (FIG. 6) that runs in a first helical direction and a second plurality 125 of sets of filaments 122 (FIG. 6) that runs in a direction intersecting the first plurality 123 of sets of filaments. As seen in FIG. 5A, each intersection of the first plurality 123 of sets of filaments with the second plurality 125 of sets of filaments may result in an arrangement of one or more cross-section geometries. At every other intersection along a particular set of either one of the first or second plurality 123, 125 of sets of filaments, the arrangement of the one or more cross-section geometries has a substantially same arrangement of cross-section geometries, and at other intersections along that set of either one of the first or second plurality 123, 125 of sets of filaments, there is a substantially different arrangement of cross-section geometries.

In one embodiment, the sets of filaments have a degree of stability and rigidity to form a tubular lattice in the relaxed state. The flexibility and stability of the tubular lattice may be such that the woven retention device 100 is able to return to an initial state from a deformed state. The deformed state may be the result of the woven retention device being in compression or tension either radially or longitudinally, and the deformation may be elastic deformation.

FIG. 5B shows one embodiment of the plurality of combinations of filament cross-section geometries forming a plurality of protuberance geometries of the retention device shown in FIG. 5A. The geometries may include, for example, shape, configuration, arrangement, and/or thickness of the filament(s) and/or the protuberance(s). For example, a first thickness 302 represents the thickness of an intersection of two filaments 310, 312, each having a relatively small thickness. In some embodiments, this intersection maybe formed by monofilament 310 overlapping another monofilament 312. In one embodiment, monofilament 310 can have a same diameter as monofilament 312. However, in another embodiment, the monofilament 310 can have a different diameter than monofilament 312.

Further, a second thickness 304 represents the thickness of an intersection of two filaments 310, 314, the filament 310 having a relatively small thickness and the filament 314 having a relatively large thickness. In some embodiments, this intersection may be formed by a multifilament (310) overlapping a monofilament (314). In another embodiment, filament 310 can be a monofilament having a smaller diameter than monofilament 314. Thus, in another embodiment, intersection 304 can be formed by a monofilament 310 overlapping a monofilament 314.

A third thickness 306 represents the thickness of an intersection of two filaments 312, 316. In an embodiment, this intersection may be formed by a multifilament (312) over a monofilament (316). In another embodiment, filament 312 can be a monofilament having a thinner diameter than monofilament 316. Thus, in another embodiment, the third thickness 306 can be formed by an intersection of monofilament 312 overlapping monofilament 316. The thicknesses 304 and 306 may have a same thickness if the filaments 310 and 312 have a same thickness, and the filaments 314 and 316 have a same thickness. Alternatively, the thicknesses of filaments 310 and 312 may be different, and the thickness of filaments 310 and 312 may be different, while the thicknesses 304 and 306 may be the same or different.

Next, a fourth thickness 308 represents the thickness between two relatively thick filaments 314, 316. In an embodiment, this intersection may be formed by a monofilament 314 overlapping a monofilament 316. Thus, each of the protuberance geometries and/or thicknesses 302, 304, 306, 308 allow for interfacing with the fastener on one side and the bone on the other side, and distributing pressure outwardly from the fastener to the bone in a distributed manner. In one embodiment, monofilament 314 can have a same diameter as monofilament 316. However, in another embodiment, the monofilaments 314, 316 can have different thicknesses.

As described above, protuberances on the interior surface of the woven retention device interface with the fastener and the protuberances of the exterior surface of the woven retention device interface with the bone surface. According to the varying protuberance thicknesses described above, the tubular lattice of the woven retention device 100 may have an outer radius spanning from a furthest outwardly extending protuberance in the radial direction on the exterior surface of the tubular lattice to a center point and/or central axis of the tubular lattice, the tubular lattice having an inner radius spanning from a furthest inwardly protruding protuberance in the radial direction on the interior surface of the tubular lattice to the center point of the tubular lattice. The tubular lattice may have an average radius that is an average between the outer radius and the inner radius. In one embodiment, the outer radius of the woven retention device 100 is greatest at the cross-section geometries that have the greatest protuberance thicknesses. Further, the inner radius of the woven retention device 100 may be the smallest at the cross-section geometries that have the largest protuberance thicknesses.

In one embodiment in a relaxed state, distributed protuberances on the exterior surface can have more than two different heights in relation to the distance from a center point of the cross-section of the tubular lattice to peaks of the distributed protuberances on the exterior surface. Further, the distributed protuberances on the exterior surface can have more than two different angles of protrusions, or amplitudes, where the amplitude of a monofilament overlying a monofilament has a higher amplitude than where the monofilament overlies a multifilament. Further, the angle, protrusion, and/or curvature of the multifilament overlying a monofilament is greater than that of a multifilament overlying a multifilament because the variance or the steepness of the curve of the multi-filament is greater. The filaments, density, and/or pick count, for example, can contribute to the difference in the sharpness, angle and/or amplitude of the protrusions. The more pronounced the protrusion, the sharper the protrusion can be considered. Various relationships between the diameter of the retention device, the thickness of the first filament(s) and, the thickness of the overlying filament(s), and the weave pattern contribute to the resulting protuberances and protuberance geometries. Varying the protuberances and protuberance geometries can provide for woven retention devices having predetermined protuberances that accommodate various bony structures. The different heights and angles of distributed protuberances on the exterior surface can allow for interdigitation with bone surfaces, especially if the bone surface is irregularly shaped.

In a second state when a fastener is inserted into the tubular lattice, pressure from the fastener can be transmitted to the tubular lattice such that at least one of (i) the heights of the protuberances on the exterior surface, (ii) the amplitudes of the protuberances on the exterior surface, and (iii) the ratio of the height to the average radius, can change to accommodate deviations in the bone surface.

FIG. 5C shows a woven patch 2005 according to an embodiment of the invention. In this embodiment, the interwoven filaments of the woven patch 200 can include a first plurality 223 of sets of filaments 220 (FIG. 6) that runs in a first direction and a second plurality 225 of sets of filaments 222 (FIG. 6) that runs in a direction intersecting the first plurality 223 of sets of filaments. As seen in FIG. 5C, each intersection of the first plurality 223 of sets of filaments with the second plurality 225 of sets of filaments may result in an arrangement of one or more cross-section geometries. At every other intersection along a particular set of either one of the first or second plurality 223, 225 of sets of filaments, the arrangement of the one or more cross-section geometries has a substantially same arrangement of cross-section geometries, and at other intersections along that set of either one of the first or second plurality 223, 225 of sets of filaments, there is a substantially different arrangement of cross-section geometries.

In one embodiment, the sets of filaments have a degree of stability and rigidity to form a lattice in the relaxed state. The flexibility and stability of the lattice may be such that the woven patch 200 is able to return to an initial state from a deformed state. The deformed state may be the result of the woven patch being in compression or tension either radially or longitudinally, and the deformation may be elastic deformation.

FIG. 5D shows a slanted sectional slice of FIG. 5C. Similar to FIG. 5B, FIG. 5D shows an embodiment of the plurality of combinations of filament cross-section geometries forming a plurality of protuberance geometries of the retention device shown in FIG. 5C. The geometries may include, for example, shape, configuration, arrangement, and/or thickness of the filament(s) and/or the protuberance(s). For example, a first thickness 402 represents the thickness of an intersection of two filaments 410, 412, each having a relatively small thickness. In some embodiments, this intersection maybe formed by monofilament 410 overlapping another monofilament 412. In one embodiment, monofilament 410 can have a same diameter as monofilament 412. However, in another embodiment, the monofilament 410 can have a different diameter than monofilament 412.

Further, a second thickness 404 represents the thickness of an intersection of two filaments 410, 414, the filament 410 having a relatively small thickness and the filament 414 having a relatively large thickness. In some embodiments, this intersection may be formed by a multifilament (410) overlapping a monofilament (414). In another embodiment, filament 410 can be a monofilament having a smaller diameter than monofilament 414. Thus, in another embodiment, intersection 404 can be formed by a monofilament 410 overlapping a monofilament 414.

A third thickness 406 represents the thickness of an intersection of two filaments 412, 416. In an embodiment, this intersection may be formed by a multifilament (412) over a monofilament (416). In another embodiment, filament 412 can be a monofilament having a thinner diameter than monofilament 416. Thus, in another embodiment, the third thickness 306 can be formed by an intersection of monofilament 412 overlapping monofilament 416. The thicknesses 404 and 406 may have a same thickness if the filaments 410 and 412 have a same thickness, and the filaments 414 and 416 have a same thickness. Alternatively, the thicknesses of filaments 410 and 412 may be different, and the thickness of filaments 410 and 412 may be different, while the thicknesses 404 and 406 may be the same or different.

Next, a fourth thickness 408 represents the thickness between two relatively thick filaments 414, 416. In an embodiment, this intersection may be formed by a monofilament 414 overlapping a monofilament 416. Thus, each of the protuberance geometries and/or thicknesses 402, 404, 406, 408 allow for interfacing with the fastener on one side and the bone on the other side, and distributing pressure outwardly from the fastener to the bone in a distributed manner. In one embodiment, monofilament 414 can have a same diameter as monofilament 416. However, in another embodiment, the monofilaments 414, 416 can have different thicknesses.

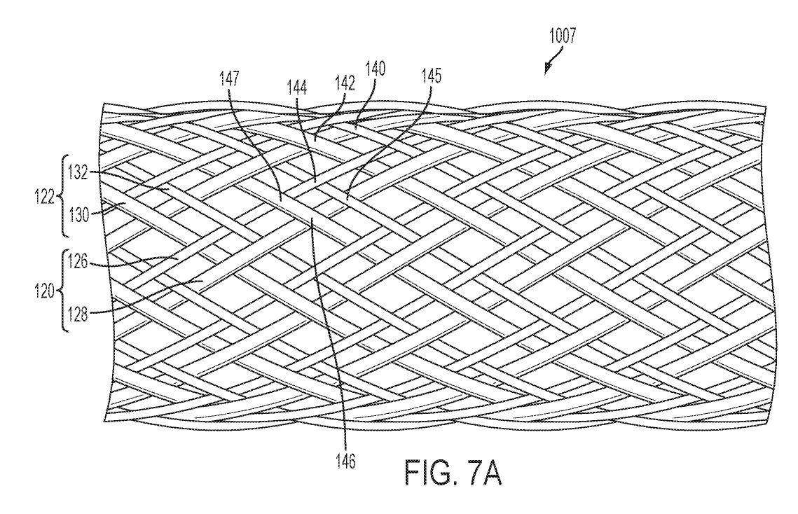

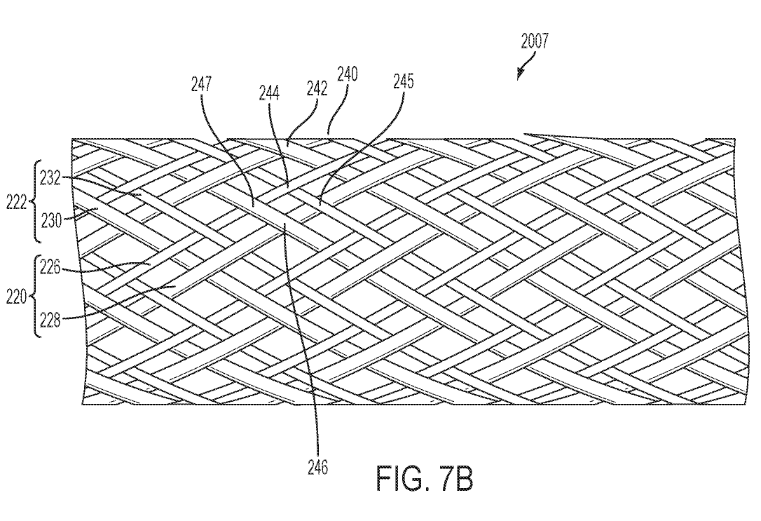

FIG. 6 shows a woven retention device according to an embodiment where a distal end 116 of the woven retention device 100 has a distal tip 115 with a first diameter D1, and the receiving portion has a second diameter D2 that is greater than the first diameter D1. In one embodiment, a diameter D2 of the proximal end 114 is substantially same as a diameter of the sleeve body 106. In contrast to FIG. 5A, the embodiment shown in FIG. 6 may have the distal end 116 tapered. For example, a set 120 of the first plurality 123 of sets of filaments includes a monofilament 126 and a monofilament 128 (as shown in FIG. 7A). A set 122 of the second plurality 125 of sets of filaments includes a monofilament 132 and a monofilament 130 (as shown in FIG. 7A).

According to an embodiment, the woven retention device 100 can include up to ten sets of filaments in each of the first and second plurality 123, 125 of sets of filaments. In another embodiment, for each of the first and second plurality 123, 125 of sets of filaments, the woven retention device 100 can include at least two sets of filaments. Thus, each of the sets of filaments may have a degree of flexibility that allows for expandability of the woven retention device 100. The filament properties and characteristics can be varied, and the number of filaments used in the weave contributes to the stability and/or rigidity of the woven retention device. For example, a small-sized woven retention device may include a half set of filaments such as 12 filament in one direction and 12 in the other direction. Whereas, a larger size may weave 24 filaments and 24 filaments. Depending upon the size of the woven retention device, a range of the quantity of filaments can vary from 2/2 to 36/36. For example, the quantity of filaments can be 8/8, 10/10, 12/12, 24/24 and/or 36/36, according to some embodiments. Additionally, other filament quantities are also possible. An even number of filaments and bobbins are contemplated, resulting in a symmetrical pattern. But an odd number of filaments can be utilized as well and would result in a non-symmetrical pattern.

FIG. 7A shows a close-up of the woven retention device 1007 according to an embodiment having a combination of different filaments. The filaments can be of different shapes and diameters. For example, the filaments can be a combination of round filaments and flat filaments, or all flat filaments, or all round filaments. The shapes of the filaments are not limited to flat and round, however, and may also include rectangular, triangular, and elliptical shapes, or other cross-section shapes. As shown in FIG. 7A, the woven retention device has flat multifilaments 142 and round monofilaments 140. In another embodiment, filament 142 can be a monofilament having a different or the same diameter as monofilament 140. In another embodiment, the flat multifilaments 142 have a larger width than height. In one embodiment, the round monofilaments 140 can have a substantially circular cross-section. In one embodiment, the round monofilaments 142 can have a substantially circular cross-section. According to some embodiments, the thickness of the monofilaments 140 is greater than the thickness of the multifilaments 142. According to the combinations of different filaments used, different types of filament intersections can be provided. For example, each of intersections 144, 145, 146, and 147 can comprise a different arrangement and/or combination of filaments, as discussed further below.

As shown in FIG. 7A, a set 120 of the first plurality 123 of sets of filaments includes a set 120 including a monofilament 126 and a monofilament 128 having different or the same diameters. In another embodiment, the set 120 of the first plurality 123 of sets of filaments can include a monofilament and a multifilament. A set 122 of the second plurality 125 of sets of filaments includes a monofilament 132 and a monofilament 130 having the same or different diameters. In another embodiment, the set 122 of the second plurality 125 of sets of filaments can include a monofilament and a multifilament.

FIG. 7B shows a close-up of the woven patch 2007 according to an embodiment having a combination of different filaments. For example, a set 220 of the first plurality 223 of sets of filaments includes a monofilament 226 and a monofilament 228 (as shown in FIG. 7B). A set 222 of the second plurality 225 of sets of filaments includes a monofilament 232 and a monofilament 230 (as shown in FIG. 7B). The filaments can be of different shapes and diameters. For example, the filaments can be a combination of round filaments and flat filaments, or all flat filaments, or all round filaments. The shapes of the filaments are not limited to flat and round, however, and may also include rectangular, triangular, and elliptical shapes, or other cross-section shapes. As shown in FIG. 7B, the woven patch has flat multifilaments 242 and round monofilaments 240. In another embodiment, filament 242 can be a monofilament having a different or the same diameter as monofilament 240. In another embodiment, the flat multifilaments 242 have a larger width than height. In one embodiment, the round monofilaments 240 can have a substantially circular cross-section. In one embodiment, the round monofilaments 242 can have a substantially circular cross-section. According to some embodiments, the thickness of the monofilaments 240 is greater than the thickness of the multifilaments 242. According to the combinations of different filaments used, different types of filament intersections can be provided. For example, each of intersections 244, 245, 246, and 247 can comprise a different arrangement and/or combination of filaments, as discussed further below.

As shown in FIG. 7B, a set 220 of the first plurality 223 of sets of filaments includes a set 220 including a monofilament 226 and a monofilament 228 having different or the same diameters. In another embodiment, the set 220 of the first plurality 223 of sets of filaments can include a monofilament and a multifilament. A set 222 of the second plurality 225 of sets of filaments includes a monofilament 232 and a monofilament 230 having the same or different diameters. In another embodiment, the set 222 of the second plurality 225 of sets of filaments can include a monofilament and a multifilament.

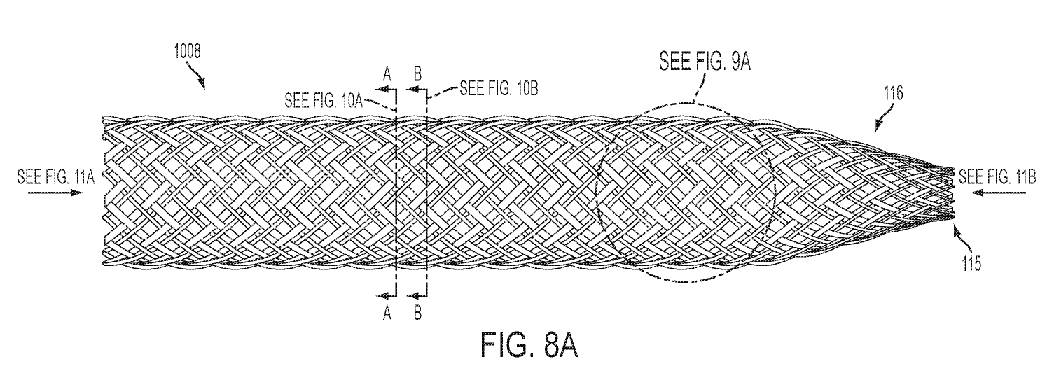

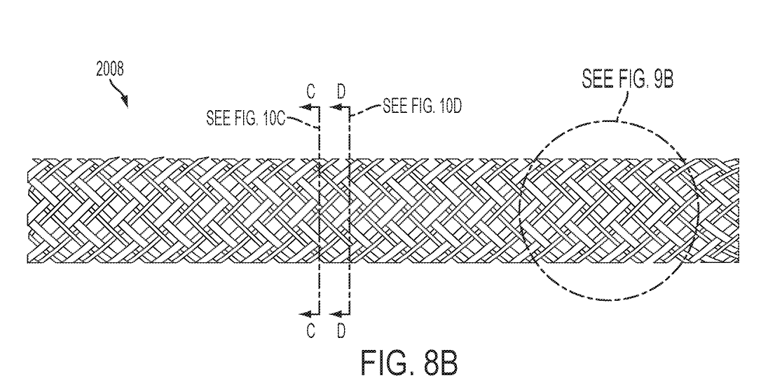

FIG. 8A shows the woven retention device 1008 with a tapered distal end 116 along its longitudinal axis.

FIG. 8B shows the woven patch 2008 with a tapered distal end 216 along its longitudinal axis.

FIG. 9A shows a close-up view of the woven retention device 1008 of FIG. 8A, according to one embodiment. As explained below, a set of filaments can include one or more filaments. In one embodiment, a set of filaments can include filaments that are side by side and the filaments including an inner filament and an outer filament. The inner filament in one embodiment can be disposed on the left of the outer filament, as viewed facing the receiving portion in a longitudinal direction. For example, FIG. 9A shows one embodiment of a woven retention device 1008, wherein each of the first plurality 123 (FIG. 5A) of sets of filaments 120 includes a first inner filament 126 and a first outer filament 128, and each of the second plurality 125 (FIG. 5A) of sets of filaments 122 includes a second inner filament 132 and a second outer filament 130. In one embodiment, one of the outer filaments and the inner filaments can be a round monofilament 140 and one of the outer filaments and the inner filaments can be a flat multifilament 142. However, in another embodiment, filament 142 can be a round monofilament with a different or a same diameter as monofilament 140. In one embodiment, the woven retention device 1008 is configured such that the plurality of interwoven filaments are comprised of alternating round monofilaments and flat multifilaments. In this embodiment, each of the sets of filaments can have a consistent and uniform order of filaments, which allows for a uniform arrangement of protuberances. In another embodiment, the plurality of interwoven filaments are comprised of alternating round monofilaments of a first diameter and round filaments of a second diameter that is greater than or less than the first diameter.

As shown in FIG. 9A, in one embodiment, the first inner filament 126 can be a flat multifilament 142, the first outer filament 128 can be a round monofilament 140, the second inner filament 132 can be a flat multifilament 142 and the second outer filament 130 can be a round monofilament 140. In another embodiment as shown in FIG. 7, the first inner filament 126 can be a round monofilament 140, the first outer filament 128 can be a flat multifilament 142 and the second outer filament 130 can be a flat multifilament 142 and the second inner filament 132 can be a round monofilament 140. In another embodiment, the first inner filament 126 can be a flat multifilament 142 and the first outer filament 128 can be a flat multifilament 142 while the second outer filament 130 can be a round monofilament 140 and the second inner filament 132 can be a round monofilament 140.

In alternate embodiments, the first inner filament 126 can be a round monofilament 142, the first outer filament 128 can be a round monofilament 140 having the same or different diameter as monofilament 142, the second inner filament 132 can be a round monofilament 142 and the second outer filament 130 can be a round monofilament 140 having a same or different diameter as round monofilament 142.

Each of the different monofilament/multifilament arrangements allow for the protuberances to occur at different regions. In FIG. 9A, the protuberances form a diamond arrangement shown by the shape defined by intersection points 144′, 145′, 146′, and 147′. For example, as shown in FIG. 7A, the first inner filament 126 and the second outer filament 130 being monofilaments results in a pronounced protuberance (e.g., a protuberance having the thickness 308 in FIG. 5B) to occur at a top intersection point 144 of a diamond arrangement of the combination of intersections, the diamond arrangement being defined by the shape outlined by intersection points 144, 145, 146, and 147. On the other hand, as shown in FIG. 9A, having the first outer filament 128 and the second outer filament 130 being monofilaments results in a pronounced protuberances to occur at a bottom 146′ of a diamond arrangement of the combination of intersections, the diamond arrangement being defined by the shape outlined by intersection points 144′, 145′, 146′, and 147′.

As can be seen from FIG. 9A, the woven retention device 1008 can be configured so that the plurality of interwoven filaments follow a two-under/two-over configuration, where each of the filaments overlie two intersecting filaments and underlie two intersecting filaments. In another embodiment, at each intersection point, a round monofilament either overlies both of the intersecting filaments or is overlain by both of the intersecting filaments and the flat multifilament overlies one of the intersecting filaments and is overlain by the other of the intersecting filaments. However, other contemplated embodiments include a one-over-one weave provided that there is sufficient rigidity and flexibility of the filaments to generate the protuberances.

FIG. 9B shows a close-up view of the woven patch 2008 of FIG. 8B, according to one embodiment. As explained below, a set of filaments can include one or more filaments. In one embodiment, a set of filaments can include filaments that are side by side and the filaments including an inner filament and an outer filament. The inner filament in one embodiment can be disposed on the left of the outer filament, as viewed facing the receiving portion in a longitudinal direction. For example, FIG. 9B shows one embodiment of a woven patch 2008, wherein each of the first plurality 223 (FIG. 5) of sets of filaments 220 includes a first inner filament 226 and a first outer filament 228, and each of the second plurality 225 (FIG. 5) of sets of filaments 222 includes a second inner filament 232 and a second outer filament 230. In one embodiment, one of the outer filaments and the inner filaments can be a round monofilament 240 and one of the outer filaments and the inner filaments can be a flat multifilament 242. However, in another embodiment, filament 242 can be a round monofilament with a different or a same diameter as monofilament 240. In one embodiment, the woven patch 2008 is configured such that the plurality of interwoven filaments are comprised of alternating round monofilaments and flat multifilaments. In this embodiment, each of the sets of filaments can have a consistent and uniform order of filaments, which allows for a uniform arrangement of protuberances. In another embodiment, the plurality of interwoven filaments are comprised of alternating round monofilaments of a first diameter and round filaments of a second diameter that is greater than or less than the first diameter.

As shown in FIG. 9B, in one embodiment, the first inner filament 226 can be a flat multifilament 242, the first outer filament 228 can be a round monofilament 240, the second inner filament 232 can be a flat multifilament 242 and the second outer filament 230 can be a round monofilament 240. In another embodiment as shown in FIG. 7B, the first inner filament 226 can be a round monofilament 240, the first outer filament 228 can be a flat multifilament 242 and the second outer filament 230 can be a flat multifilament 242 and the second inner filament 232 can be a round monofilament 240. In another embodiment, the first inner filament 226 can be a flat multifilament 242 and the first outer filament 228 can be a flat multifilament 242 while the second outer filament 230 can be a round monofilament 240 and the second inner filament 232 can be a round monofilament 240.

In alternate embodiments, the first inner filament 226 can be a round monofilament 242, the first outer filament 228 can be a round monofilament 240 having the same or different diameter as monofilament 242, the second inner filament 232 can be a round monofilament 242 and the second outer filament 230 can be a round monofilament 240 having a same or different diameter as round monofilament 242.

Each of the different monofilament/multifilament arrangements allow for the protuberances to occur at different regions. In FIG. 9B, the protuberances form a diamond arrangement shown by the shape defined by intersection points 244′, 245′, 246′, and 247′. For example, as shown in FIG. 7B, the first inner filament 226 and the second outer filament 230 being monofilaments results in a pronounced protuberance (e.g., a protuberance having the thickness 308 in FIG. 6) to occur at a top intersection point 144 of a diamond arrangement of the combination of intersections, the diamond arrangement being defined by the shape outlined by intersection points 244, 245, 246, and 247. On the other hand, as shown in FIG. 9B, having the first outer filament 228 and the second outer filament 230 being monofilaments results in a pronounced protuberances to occur at a bottom 246′ of a diamond arrangement of the combination of intersections, the diamond arrangement being defined by the shape outlined by intersection points 244′, 245′, 246′, and 247′.

As can be seen from FIG. 9B, the woven patch 2008 can be configured so that the plurality of interwoven filaments follow a two-under/two-over configuration, where each of the filaments overlie two intersecting filaments and underlie two intersecting filaments. In another embodiment, at each intersection point, a round monofilament either overlies both of the intersecting filaments or is overlain by both of the intersecting filaments and the flat multifilament overlies one of the intersecting filaments and is overlain by the other of the intersecting filaments. However, other contemplated embodiments include a one-over-one weave provided that there is sufficient rigidity and flexibility of the filaments to generate the protuberances.

For FIGS. 9A and 9B, alternative weaving patterns besides the two-over/two-under configuration are also contemplated within the broad inventive principles disclosed herein. A one-over/one-under configuration is contemplated where each filament alternatingly overlies and underlies an intersecting filament. In one embodiment, a three-over/three-under weave pattern is contemplated where each filament overlies three intersecting filaments before underlying three intersecting filaments. In another embodiment, a two-over/one-under is contemplated where each filament overlies two intersecting filaments and then underlies one intersecting filament. Alternatively, a one-over/two-under arrangement is also possible where a filament overlies one intersecting filament before underlying two intersecting filaments. In another embodiment, a three-over/one-under is contemplated where each filament overlies three intersecting filaments and then underlies one intersecting filament. Alternatively, a one-over/three-under arrangement is also possible where a filament overlies one intersecting filament before underlying three intersecting filaments. With each of these weaving patterns, sufficient stability, rigidity, compressibility, sheer strength, and/or tensile strength can allow for the pressure from the fastener is able to transmit force in a distributed manner to the bone surface.

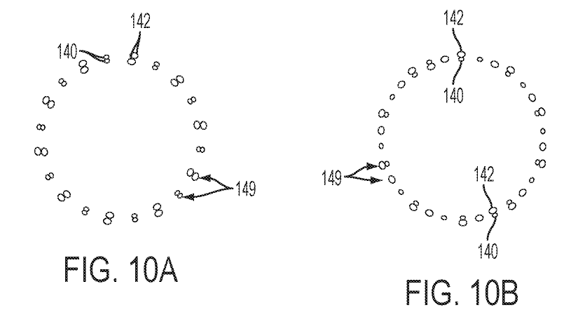

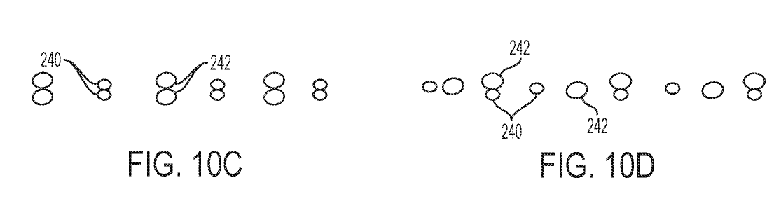

FIGS. 10A and 10B show cross sections showing the intersecting filaments of the woven retention device 1008, representing various cross-sectional geometries 149 at the sections A-A and B-B indicated in FIG. 8A. The woven device 1008 can be configured as shown such that the intersecting sets of filaments form a plurality of cross-sectional geometries and/or thicknesses. In FIG. 10A, section A-A of FIG. 8A, the large round over round grouping represents the intersection of a relatively large round monofilament 142 over another relatively large round monofilament 142. The cross-sections of these relatively large round monofilaments 142, 142 can be the same or have different thicknesses from each other. On the other hand, the smaller round-over-round grouping represent a relatively small round monofilament 140 over another relatively small round monofilament 140 intersection where the round monofilaments 140, 140 can have the same or different thicknesses from each other. In FIG. 10B, section B-B of FIG. 8A, the large circle over the small circle grouping represents a round monofilament 142 over a round monofilament 140 where the diameter of round monofilament 142 is greater than round monofilament 140. Further, a small circle over large circle cross-section geometry represents a round monofilament 140 over a round monofilament 142 where the diameter of the round monofilament 140 is smaller than round monofilament 142.

The round monofilaments of the woven retention device can have differing diameters. In one embodiment, the round monofilaments can have a diameter in a range of about 0.1 mm-0.4 mm. In one embodiment, the round monofilament of the woven retention device is 0.2 mm.

The multifilaments of the woven retention device according to some embodiments can have various thicknesses and widths. For example, a multifilament may have a thickness of less than 0.1 mm. The cross-sectional shape, e.g., flat or round, and the texture, for example, of the multifilaments can also be relevant. The number of filaments and pattern can also be relevant. As such, with those considerations, various filament linear mass densities can be contemplated. For example, the multifilaments can have a linear mass density in a range of about 150-250 denier. In one embodiment, the multifilaments can have a linear mass density of about 200 denier.

The woven retention device can be configured such that the intersecting sets of filaments form a plurality of differently shaped and differently sized apertures. In one embodiment, as shown in FIG. 9A, the first inner and outer filaments of one set of first filaments can be grouped closer to each other than the other sets of first filaments. Likewise, the second inner and outer filaments of one set of second filaments can be grouped closer to each other than the other sets of second filaments. When the two sets of filaments intersect, as shown in FIG. 9, the area which is outlined by the first and second plurality of sets of filaments is a plurality of differently shaped and differently sized apertures 148. By having differently shaped and sized apertures, a more conducive environment for non-uniform bony surface can allow for ingrowth of bone to occur. Additionally, improved interdigitation with the bony structure can be achieved with a combination of the apertures and protuberances.

FIGS. 10C and 10D show cross sections showing the intersecting filaments of the woven patch 2008, representing various cross-sectional geometries 249 at the sections A-A and B-B indicated in FIG. 8B. The woven device 2008 can be configured as shown such that the intersecting sets of filaments form a plurality of cross-sectional geometries and/or thicknesses. In FIG. 10C, section A-A of FIG. 8B, the large round over round grouping represents the intersection of a relatively large round monofilament 242 over another relatively large round monofilament 242. The cross-sections of these relatively large round monofilaments 242, 242 can be the same or have different thicknesses from each other. On the other hand, the smaller round-over-round grouping represent a relatively small round monofilament 240 over another relatively small round monofilament 240 intersection where the round monofilaments 240, 240 can have the same or different thicknesses from each other. In FIG. 10D, section B-B of FIG. 8B, the large circle over the small circle grouping represents a round monofilament 242 over a round monofilament 240 where the diameter of round monofilament 242 is greater than round monofilament 240. Further, a small circle over large circle cross-section geometry represents a round monofilament 240 over a round monofilament 242 where the diameter of the round monofilament 240 is smaller than round monofilament 242.

The round monofilaments of the woven patch can have differing diameters. In one embodiment, the round monofilaments can have a diameter in a range of about 0.1 mm-0.4 mm. In one embodiment, the round monofilament of the woven patch is 0.2 mm.

The multifilaments of the woven patch according to some embodiments can have various thicknesses and widths. For example, a multifilament may have a thickness of less than 0.1 mm. The cross-sectional shape, e.g., flat or round, and the texture, for example, of the multifilaments can also be relevant. The number of filaments and pattern can also be relevant. As such, with those considerations, various filament linear mass densities can be contemplated. For example, the multifilaments can have a linear mass density in a range of about 150-250 denier. In one embodiment, the multifilaments can have a linear mass density of about 200 denier.

In an embodiment where the woven patch 200 is applied to a bone dowel, an allograph material may reincorporate over time the size of the openings. The woven patch 200 may be provided around a bony structure or structures to contain the pieces and allow for regeneration. Thus, the woven patch may provide an interface for allowing bony tissue to grow through it, and can enhance fixation of the encapsulated material to prevent the pieces from moving around. The woven patch thus can allow in one embodiment a natural healing process for bone tissue. The woven patch can be made of fiberglass or carbon fiber and can serve as an epoxy for reinforcement strength. For example, the woven patch can provide an interface so that the epoxy has a chance to not migrate while the bone tissue undergoes healing. In one embodiment, the woven patch can enable putting in a bone dowel in and ensuring that it is not going to migrate because of an improved interface that does not arrest the healing process or arrest the bone formation process. As an example, a bone void filler can ensure that the final construct needs to stay without slowing down or altering the normal metabolic healing process. The bone void filler in this example is not meant as an element to structural integrity to give the resulting construct more rigidity—instead the bone void filler can act like a carbon fiber piece of epoxy.

The woven patch 200 can be configured such that the intersecting sets of filaments form a plurality of differently shaped and differently sized interstices. In one embodiment, as shown in FIG. 9B, the first inner and outer filaments of one set of first filaments can be grouped closer to each other than the other sets of first filaments. Likewise, the second inner and outer filaments of one set of second filaments can be grouped closer to each other than the other sets of second filaments. When the two sets of filaments intersect, as shown in FIG. 9B, the area which is outlined by the first and second plurality of sets of filaments is a plurality of differently shaped and differently sized interstices 248. By having differently shaped and sized interstices, a more conducive environment for non-uniform bony surface can allow for ingrowth of bone to occur. Additionally, improved interdigitation with the bony structure can be achieved with a combination of the interstices and protuberances. The closure of an end can be made (i.e., tip can be made) via knitting, energy (heat stake, laser, optical, ultrasound energy to melt fibers), and chemical (glue, or superglue).

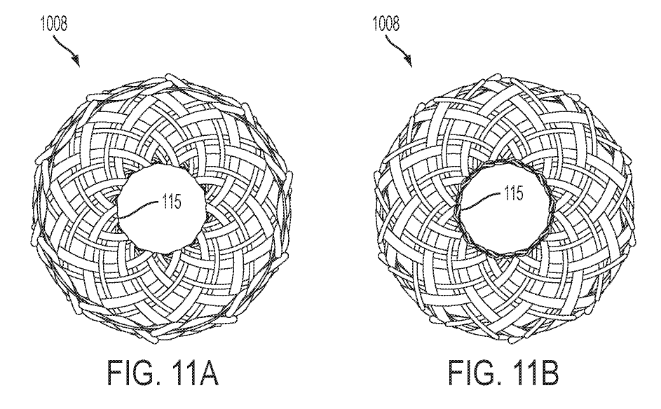

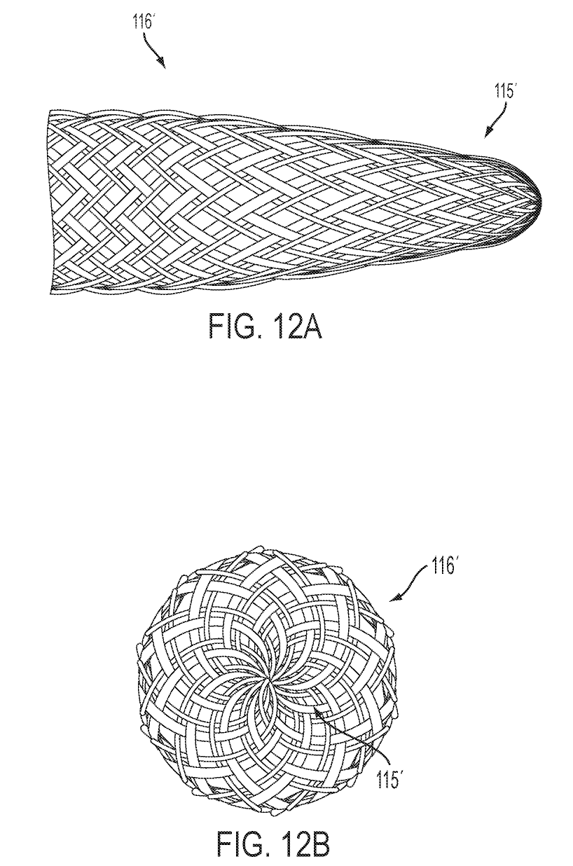

The tapering end portion can be seen from FIGS. 11A and 11B a front and rear axial direction of the woven retention device 1008, as indicated in FIG. 8. An edge of the distal tip 115 can be seen in FIGS. 11A and 11B from inner and outer sides, respectively, the distal end 116 (see FIG. 8). The tapered end can be used to facilitate inserting the woven retention device 1008 into a bone hole. In one embodiment, the tapered end can have at least a portion of the end be closed. As shown in FIGS. 12A and 12B, a distal end 116′ of a woven retention device according to another embodiment has a distal tip 115′ that can be closed to further allow for a push rod to push the woven retention device 100 into the hole. The closure of the end can be made (i.e., tip can be made) via knitting, energy (heat stake, laser, optical, ultrasound energy to melt fibers), and chemical (glue, or superglue).

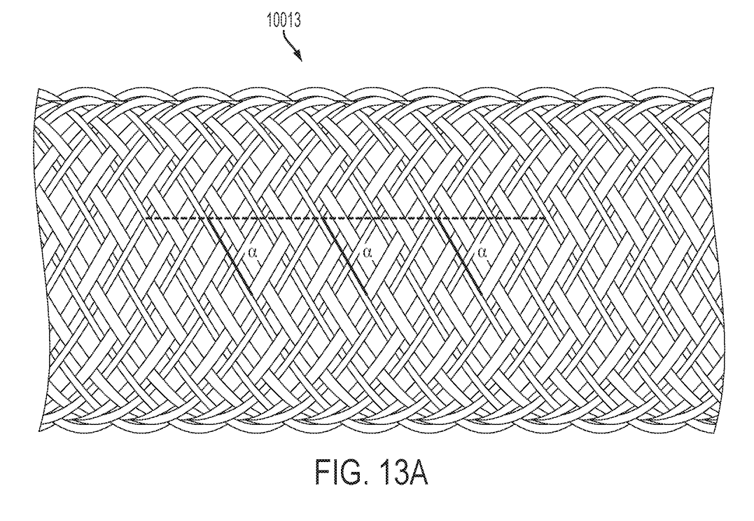

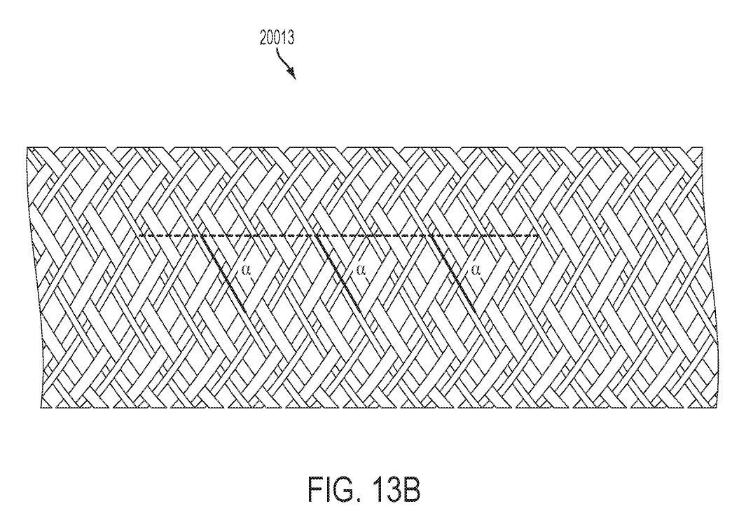

In FIG. 13A, the interwoven filaments of a woven retention device 10013 extend around the tubular lattice in an angle range of α. In one embodiment, α can represent a range from about 40-60 degrees with respect to a longitudinal direction of the woven retention device. In another embodiment, a can represent a range from about 15-75 degrees with respect to a longitudinal direction of the body sleeve. In one embodiment, α represents 45 degrees. The retention device can, in the relaxed state, have the interwoven filaments that extend around the tubular lattice at about a 45 degree angle with respect to a longitudinal direction of the woven retention device. The configuration and angle α shown in FIG. 13A can correspond to a relaxed state of the woven retention device 100 according to some embodiments.

In FIG. 13B, the interwoven filaments of a woven patch 20013 extend around the lattice in an angle range of α. In one embodiment, α can represent a range from about 40-60 degrees with respect to a parallel direction of an exterior or interior surface of the woven patch. In another embodiment, α can represent a range from about 15-75 degrees with respect to a longitudinal direction of the body sleeve. In one embodiment, α represents 45 degrees. The patch can, in the relaxed state, have the interwoven filaments that extend around the lattice at about a 45 degree angle with respect to a parallel direction of the woven patch. The configuration and angle α shown in FIG. 13B can correspond to a relaxed state of the woven patch 200 according to some embodiments.

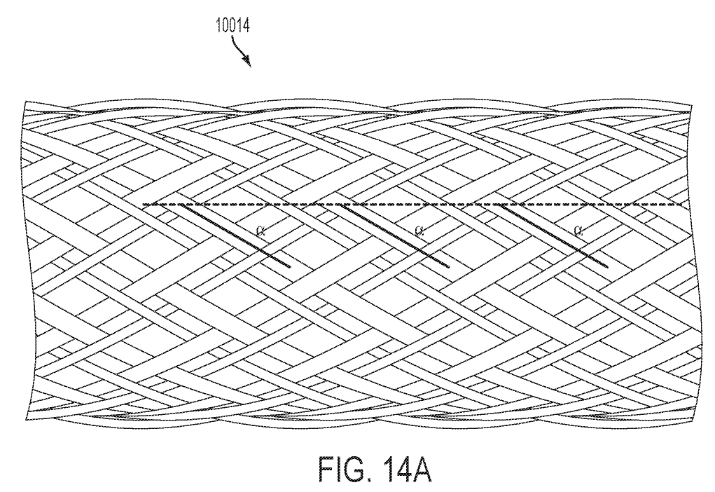

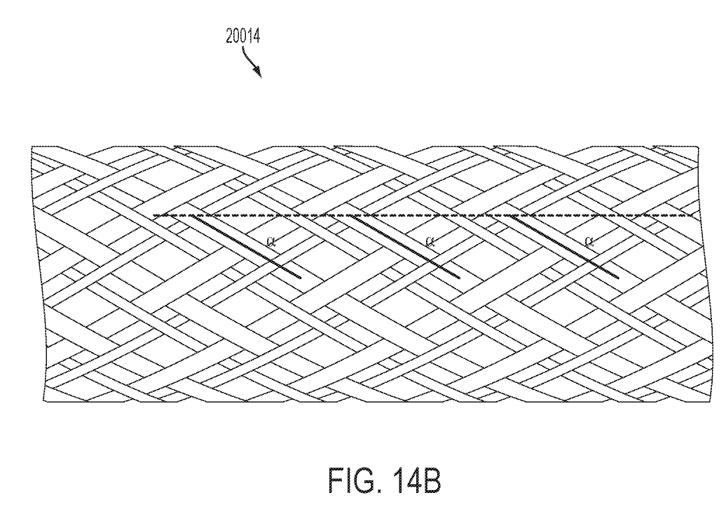

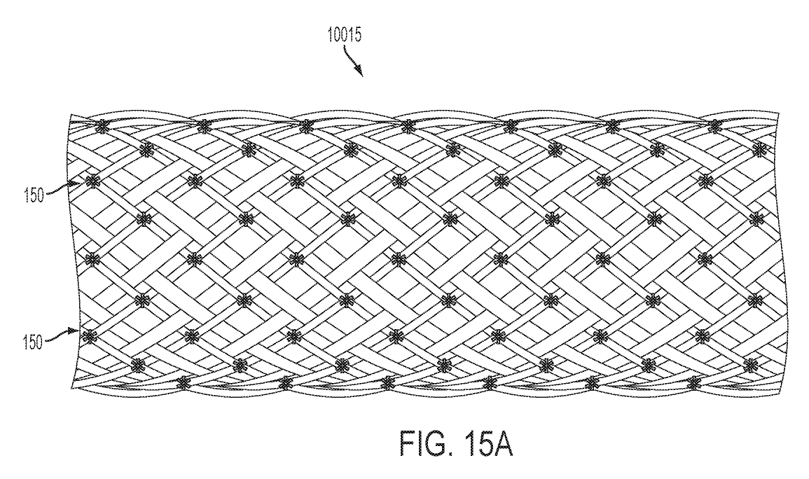

FIG. 14A shows that the braid angle α for a woven retention device 10014, according to another embodiment. The braid angle α can be smaller than 45 degrees. The configuration and braid angle α in FIG. 14A can also represent the woven retention device 10013 of FIG. 13A in a constricted state or if the woven retention device 10014 has a predetermined diameter lower than a predetermined value and the filaments exceed a predetermined thickness. For example, when the woven retention device has an average diameter of 2 mm, the braid angle α can be about 35 degrees.

FIG. 14B shows that the braid angle α for a woven patch 20014, according to another embodiment. The braid angle α can be smaller than 45 degrees. The configuration and braid angle α in FIG. 14B can also represent the woven patch 20013 of FIG. 13B in a constricted state or if the woven patch 20014 has a predetermined diameter lower than a predetermined value and the filaments exceed a predetermined thickness. For example, when the woven patch has an average diameter of 2 mm, the braid angle α can be about 35 degrees.

A woven patch 200 for interfacing with a bone surface can thus include a sleeve body comprising a plurality of sets of interwoven filaments that form a two-dimensional lattice with a plurality of protuberances distributed on an interior surface and an exterior surface of the lattice at a predetermined spatial relationship. The plurality of sets of interwoven monofilaments can have a plurality of different diameters, and the sleeve body can be configured to surround at least a portion of a fastener.

The woven patch 200 can have a first end that is configured to interface with at least a portion of the fastener and a second end that is opposite of the first end to the sleeve body.

In a first state, the sleeve body can have a plurality of combinations of filament cross-section geometries at intersection points of the interwoven filaments, the plurality of combinations of filament cross-section geometries forming a plurality of protuberance thicknesses. A thickness of each protuberance can be measured in a direction as a thickness of the sleeve body. In a second state when a fastener is inserted into or applied to the lattice, pressure from the fastener can be transmitted to the lattice such that the spatial relationship of the protuberances changes according to a function of bone density and according to a function of an interfacing surface shape of the fastener.

The interwoven filaments can extend across the lattice at an angle of about 45 degrees with respect to a length of the woven patch. The distributed protuberances are arranged in a diamond-shaped pattern grid. A length of the sleeve body is in a range from about 10 mm to 100 mm.

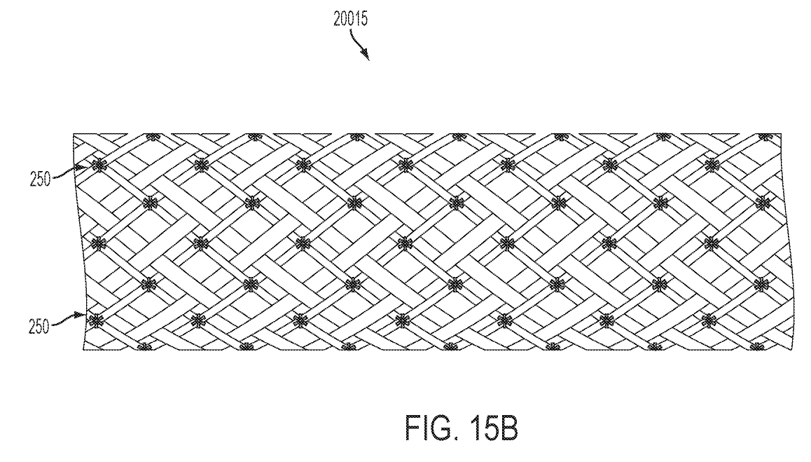

FIG. 15A shows the distributed protuberances 150 on the exterior surface of the woven retention device 10015 according to an embodiment. The woven retention device 10015 can allow for a different loading pattern (dynamic load) than the screw because of uniform radial pressure. Instead of pushing or cutting bone, the screw can push on and deform the woven structure of the woven retention device 10015, which allows for a distributed force. Preferably, the woven structures can be of a strength to not be cut or broken by the screw. The interface can be in random or patterned contacts on the exterior surface and the interior surface. For example, FIG. 15A shows that the protuberances 150 are in a substantially diamond-shaped pattern grid distributed across the tubular lattice.

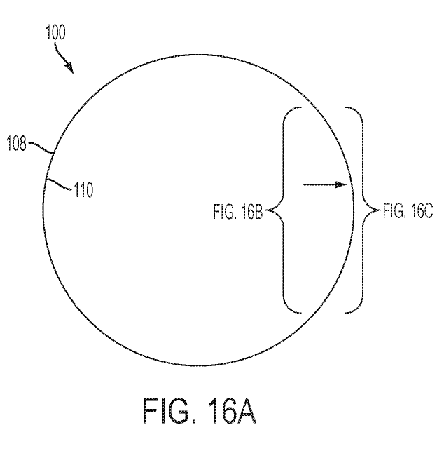

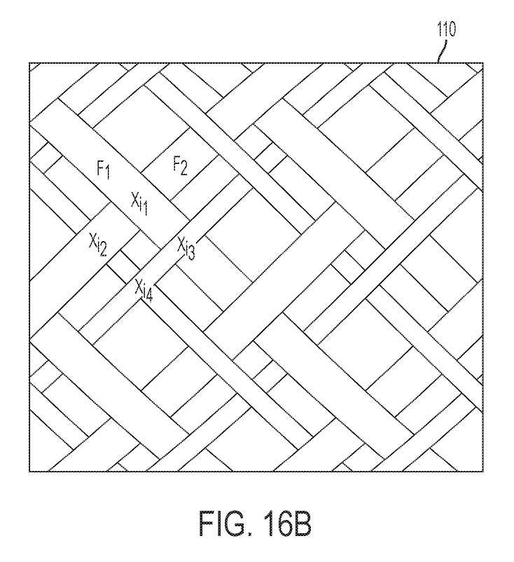

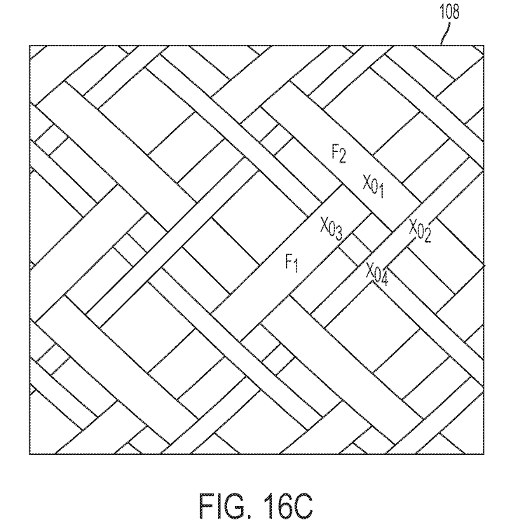

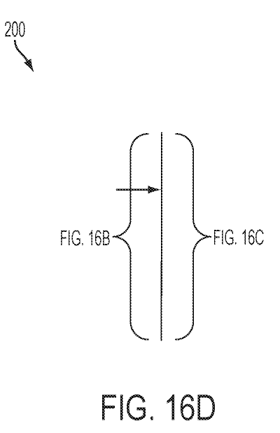

FIG. 16A is a cross-sectional schematic of the retention device (in the shape of a general circle) having an arrow representing a point of pressure contact on the interior surface 110 of the woven retention device 100. FIG. 16B represents a portion of the interior surface 110 covering the bracketed portion of FIG. 16A as viewed from inside the woven retention device 100. FIG. 16C represents a reverse view from that of FIG. 16B, and thus shows an exterior surface as evidenced by bracket in FIG. 16A as viewed from outside of the woven retention device 100 looking in to the radial center. Intersections points Xi1, Xi2, Xi3, and Xi4 on the interior surface 110 in FIG. 16B respectively correspond to intersection points Xo1, Xo2, Xo3, and Xo4 on the exterior surface 108 in FIG. 16C.

As can be seen from FIGS. 16B and 16C, even though the left to right portions of the portion correspond to different regions of the woven retention device, the configuration of each portion can appear the same. That is, while an over/under weave on a left side of the interior surface can correspond to a under/over weave on a right side of the exterior surface, the left portion in a similar position as the left portion of the interior surface can resemble a similar configuration of the over/under weave. Each of the four Xi regions in FIG. 16B corresponds to a protuberance on the interior surface, and the filaments F1 and F2 can correspond to intersecting multifilaments. When viewed from the exterior surface, this same portion shows that the pressure is located on the right side of the portion. As can be seen, on the interior surface, filament F1 overlies filament F2 at intersection Xi1, whereas on the exterior surface filament F2 overlies filament F2 at intersection Xo1. However, in the left portion of the exterior surface, it resembles the left portion of the interior portion.

FIG. 15B shows the distributed protuberances 150 on the exterior surface of the woven patch 10015 according to an embodiment. The woven patch 20015 can allow for a different loading pattern (dynamic load) than the screw because of uniform radial pressure. Instead of pushing or cutting bone, the screw can push on and deform the woven structure of the woven patch 20015, which allows for a distributed force. Preferably, the woven structures can be of a strength to not be cut or broken by the screw. The interface can be in random or patterned contacts on the exterior surface and the interior surface. For example, FIG. 15B shows that the protuberances 250 are in a substantially diamond-shaped pattern grid distributed across the tubular lattice.

FIG. 16D is a cross-sectional schematic of the patch (in the shape of a general flat plane) having an arrow representing a point of pressure contact on the interior surface 210 of the woven patch 100. While FIG. 16B has so far been used to depict an interior surface of a cylindrical woven retention device, FIG. 16B can also be used to represent a portion of the interior surface 110 covering the bracketed portion of FIG. 16D as viewed from inside the woven patch 100. Similarly, FIG. 16C can represent a reverse view from that of FIG. 16B, and thus shows an exterior surface as evidenced by bracket in FIG. 16D as viewed from outside of the woven patch 200 looking in to the radial center. Intersections points Xi1, Xi2, Xi3, and Xi4 on the interior surface (shown as 110 in FIG. 16B) respectively correspond to intersection points Xo1, Xo2, Xo3, and Xo4 on the exterior surface (shown as 108 in FIG. 16C).

As can be seen from FIGS. 16B and 16C, even though the left to right portions of the portion correspond to different regions of the woven patch, the configuration of each portion can appear the same. That is, while an over/under weave on a left side of the interior surface can correspond to a under/over weave on a right side of the exterior surface, the left portion in a similar position as the left portion of the interior surface can resemble a similar configuration of the over/under weave. Each of the four Xi regions in FIG. 16B corresponds to a protuberance on the interior surface, and the filaments F1 and F2 can correspond to intersecting multifilaments. When viewed from the exterior surface, this same portion shows that the pressure is located on the right side of the portion. As can be seen, on the interior surface, filament F1 overlies filament F2 at intersection Xi1, whereas on the exterior surface filament F2 overlies filament F2 at intersection Xo1. However, in the left portion of the exterior surface, it resembles the left portion of the interior portion.

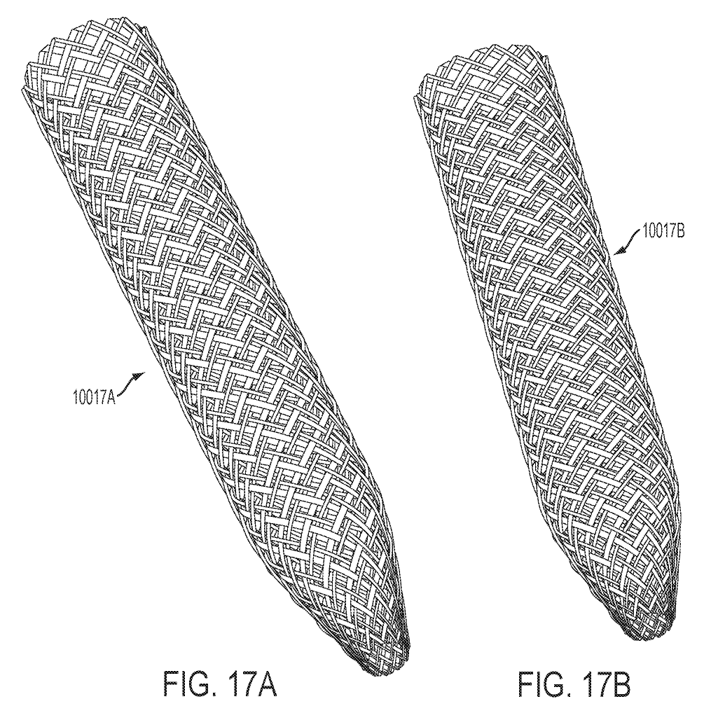

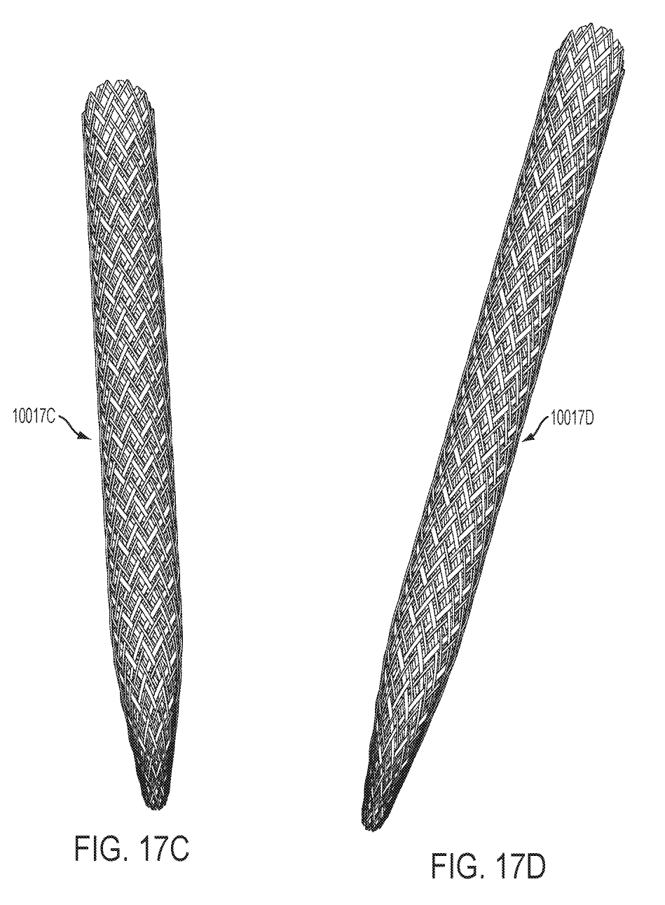

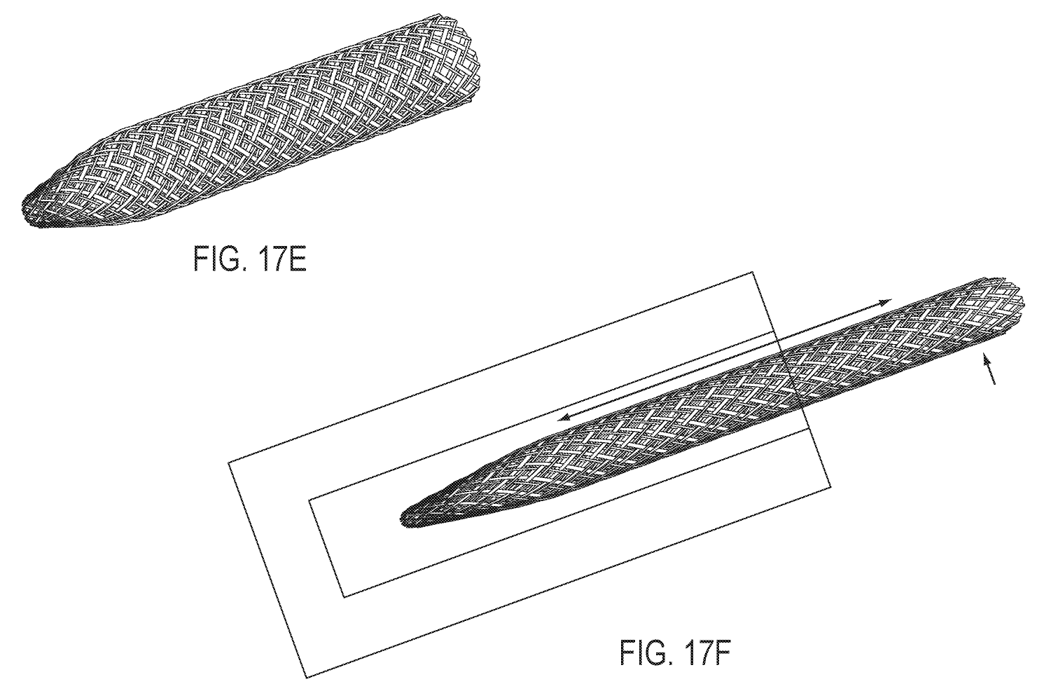

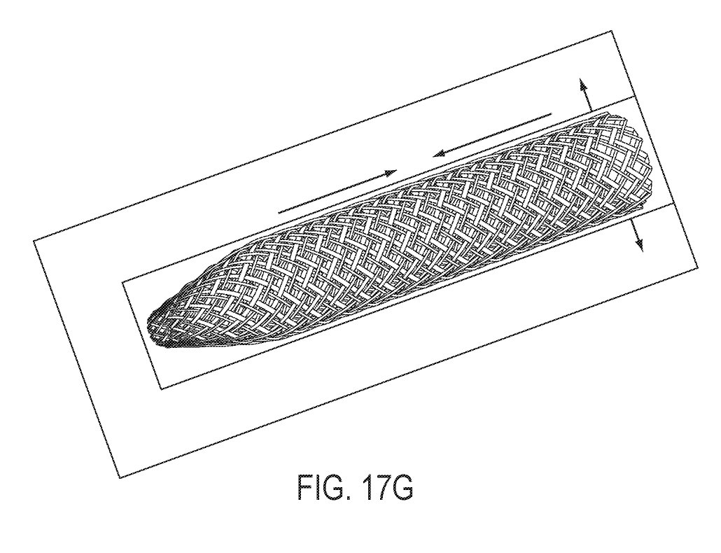

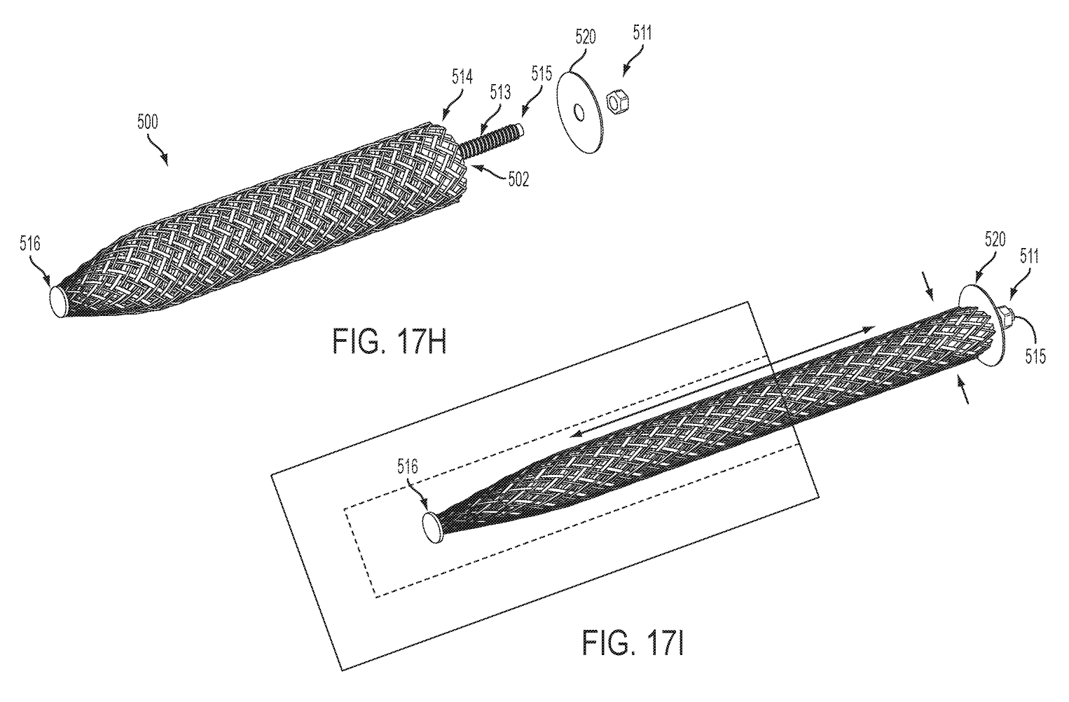

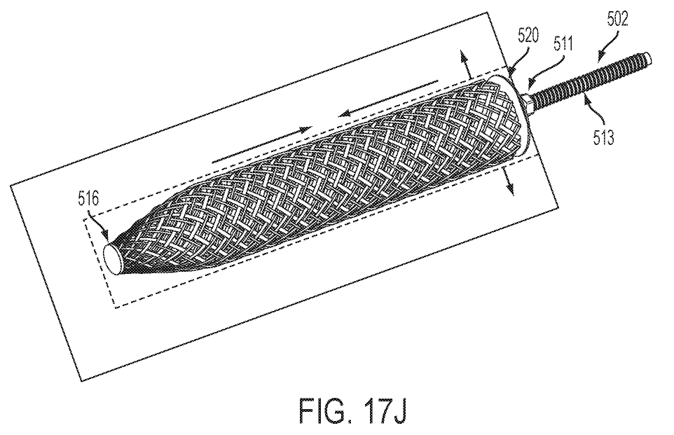

In a relaxed state, the woven retention device can be of various lengths and diameters. FIGS. 17A and 17B show that two differing lengths of embodiments of woven retention devices 10017A and 10017B, respectively. In one embodiment, the woven retention device can have a length in a range of about 30 mm to 40 mm. The length of the woven retention device can come in dynamically cuttable; and/or predetermined length, such as small—30 mm; medium—40 mm, large—40 mm, and other sizes (or ranges) are also possible. FIGS. 17C and 17D show two embodiments of woven retention devices 10017C and 10017D with differing lengths and each with a diameter that is different from FIGS. 17A and 17B. In one embodiment, the woven retention device can have a diameter of about 1.5 mm to 9.0 mm. The diameter of the woven retention device can come in predetermined sizes, such as (i) small: 2.0 mm fine (can accommodate 1.3 mm to a little over 2.0 mm pilot hole diameter and can fit 2.0 mm-2.7 mm screws); (ii) medium: 3.5 mm-6.0 mm course (can accommodate 2.4 mm to a little over 3.2 mm pilot hole diameters and can fit 3.5-6 mm screws); and (iii) large: 6.5 mm-9 mm very course (can accommodate 4.1 mm to a little over 5.9 mm pilot hole diameters and can fit 6.5-9.0 mm screws).

FIG. 17E shows a woven retention device 100 in a relaxed state. FIG. 17F shows that applying pressure in a longitudinal direction stretches the woven retention device 100 such that the diameter of the woven retention device 100 decreases. In this manner, the woven retention device 100 can be easily inserted into a bone hole 101. FIG. 17G shows that once inside the bone hole 101, the woven retention device 100 can have longitudinal forces applied to return the woven retention device 100 to a less constricted shape. In this manner, it allows for the woven retention device to snugly fit into the bone hole 101. In a relaxed state, the braid angles of the interwoven filaments can be larger than the braid angle in a construed or elongated state. In one embodiment, the retention device allows for a maximum distribution of protuberances based on the braid angle. Thus, the retention device 100 in the elongated state of FIG. 17F can have less distributed protuberances than the retention device 100 in the less constricted state of FIG. 17G.

In a relaxed state, the woven patch can be of various lengths and diameters. In one embodiment, the woven patch can have a length in a range of about 10 mm to 100 mm. In an embodiment, the woven patch can have a length in a range of about 30 mm to 40 mm. The length of the woven patch can come in dynamically cuttable; and/or predetermined length, such as small—30 mm; medium—40 mm, large—40 mm, and other sizes (or ranges) are also possible.

Next, the woven patch can be used as a patch for a pedicle breach or in supporting a pedicle breach, which is insulation for potential nerve breach in cases of non-benign apertures in the pedicle. This can serve as filling in the gaps between bone and a fastener to avoid nerve damage or discomfort. In one embodiment, the woven patch can encapsulate the vertebrae bone, including one or more pedicle portions. The patch can be used for bone sub-support in structural scaffolding of bone that is in need of strengthening. A woven patch can support bone in a certain shape or manner. A biotextile patch can be used to surround a bone to give it support instead of a classical stainless steel cable.

Embodiments of the invention are not limited to being used in any particular bone, and may be configured for use in any bone.

As shown in FIGS. 17H-J, one embodiment of the invention is directed to a retention device that can have an inner layer that interfaces with screw for maximum contact with thread and an outer layer that interfaces with bone for maximum interdigitation with bone. The retention device can include a fastener attached to one end of the body such that when the fastener is moved inside the hollow tube structure, a compressive force is exerted on the implantable retention device by the fastener in a direction parallel to a longitudinal axis of the hollow tube structure. In this way, the compressive force radially expands the hollow tube structure to an expanded state. In one embodiment, the fastener can be inserted a predetermined distance into the hollow tube structure, and the proximal end of the implantable retention device can be configured to detach from the fastener.

In one embodiment, the sleeve can radially expand and contract from a first state to a second state. The first state has a constricted diameter and the second state is a uniformly expanded middle portion. A smooth surface for the inner layer interface has application in soft tissue. Biotextile technology can be used for soft tissue reapproximation or repair of any muscoskeletal injury. A biofabric can be used for other means of fixation. The smooth surface may be used in a Chinese finger trap functionality.

The sleeve can work at filling the hole better to provide more points of contact for the bone interface. One way it can do so is by having two sleeves nested, which can add additional advantages using the multiple points of contact interface. It can also have a homogeneous and uniform interface for screw engagement so that a number of characteristics of the sleeve can be achieved: Rigidity, Compressibility, Stability, Sheer strength (at a predetermined level), Tensile strength (at a predetermined level). The implantable retention device can be made of at least one of silk, non-woven felt, and collagen.

The implantable retention device can be made such that the body further includes a plurality of engagement sites on the exterior and interior surfaces of the body that interact with a surface of the bone tissue and a surface of the fastener, respectively. In one embodiment, the plurality of engagement sites can exert a plurality of retaining forces on the surface of the bone and the surface of the fastener to resist removal of the fastener from the implantable retention device and to resist removal of the implantable retention device from the bone. In one embodiment, the implantable retention device can be configured such that when an engagement site fails to exert a sufficient retaining force, others of the plurality of points of contact compensate for the failed engagement site. In one embodiment, the engagement sites comprise elements that are raised relative to the exterior and interior surfaces of the body.

The inner surface could be configured to interface with different structures besides a screw (clamp, smooth, roughened) to provide a strong connection as long as there are many points of contact to provide sufficient sheer strength and a monolithic structure (that is, if one point fails, whole structure does not fail). Thus, in one embodiment, the implantable retention device can be monolithic.

In one embodiment, the implantable retention device 500 has a shear strength along an axis substantially orthogonal to a longitudinal axis of the hollow tube structure such that the hollow tube structure resists shearing from surface areas of the bone tissue and the fastener up to a predetermined threshold when the fastener is inserted into the bone hole.

Further, the degree of flexibility of the implantable retention device can allow for the hollow tube structure to change to a contorted state when the fastener is inserted. In one embodiment, the contorted state can be different than the resting state, the constricted state and the expanded state. In an embodiment of the contorted state of the implantable retention device, at least portions of the body interdigitate with variations in a surface of the fastener 502.

In one embodiment, a tensile force applied to the hollow tube structure parallel to a longitudinal axis of the hollow tube structure causes the hollow tube structure to radially constrict and the compressive force applied to the hollow tube structure parallel to the longitudinal axis of the hollow tube structure causes the hollow tube structure to expand.

In one embodiment, as shown in FIG. 17H, a screw-activated device can be attached to the implantable retention device 500. The sleeve body can be configured to expand. The woven retention device 500 can further include a screw-activated device including i) a screw 502 having a threaded portion 513 and ii) a bolt 511 that is configured to be threaded along the threaded portion 513 of the screw 502.

A first end of the screw can be attached to the distal end 516 of the woven retention device and at least a portion of the threaded portion 513 can run along a longitudinal direction of the body inside the woven retention device 500. A second end 515 of the screw 502 is configured to accept the bolt 511 such that when the bolt is moved inside the tubular structure, a compressive force is exerted on the woven retention device by the bolt in a direction parallel to a longitudinal axis of the tubular structure.

The compressive force radially can expand the tubular structure to the expanded state. When the fastener 502 is inserted a predetermined distance into the tubular structure, the proximal end 514 of the woven retention device can be configured to detach from the fastener 502.

The screw-activated device can include a screw 502 and a bolt 511 that is configured to be threaded along threads 513 of the screw 502. The implantable retention device can further include the screw-activated device, where a distal end portion 516 of the screw-activated device is attached to the distal end of the implantable retention device. In one embodiment, when the bolt 511 is moved along the screw 502 inside the hollow tube structure, a compressive force is exerted on the implantable retention device by the screw-activated device in a direction parallel to a longitudinal axis of the hollow tube structure, the compressive force radially expanding the hollow tube structure to the expanded state. In one embodiment, a washer 520 can be used to interface with the implantable retention device and the screw. For example, the washer 520 may be attached to the proximal end of the implantable retention device such that when the screw 502 is moved along the threaded portion 513, the implantable retention device can uniformly expand from uniform pressure from the washer 520.

In one embodiment, the implantable retention device can include a screw-activated device including i) a screw 502 having a threaded portion 513 and ii) a bolt 511 that is configured to be threaded along the threaded portion 513 of the screw 502. In one embodiment, a first end 516 of the screw can be attached to the distal end portion 516 at the distal end of the implantable retention device 500 and at least a portion of the threaded portion 513 runs along a longitudinal direction of the body inside the implantable retention device 500. A second end 515 of the screw 502 can be configured to accept the bolt 511. When the bolt 511 is moved inside the hollow tube structure, as shown in FIG. 17J, a compressive force can be exerted on the implantable retention device 500, for example through washer 520, by the bolt 511 in a direction parallel to a longitudinal axis of the hollow tube structure, and the compressive force can radially expand the hollow tube structure to the expanded state.

In one embodiment, when the bolt has been inserted a predetermined distance into the hollow tube structure, the distal end of the implantable retention device can be configured to detach the screw-activated device.

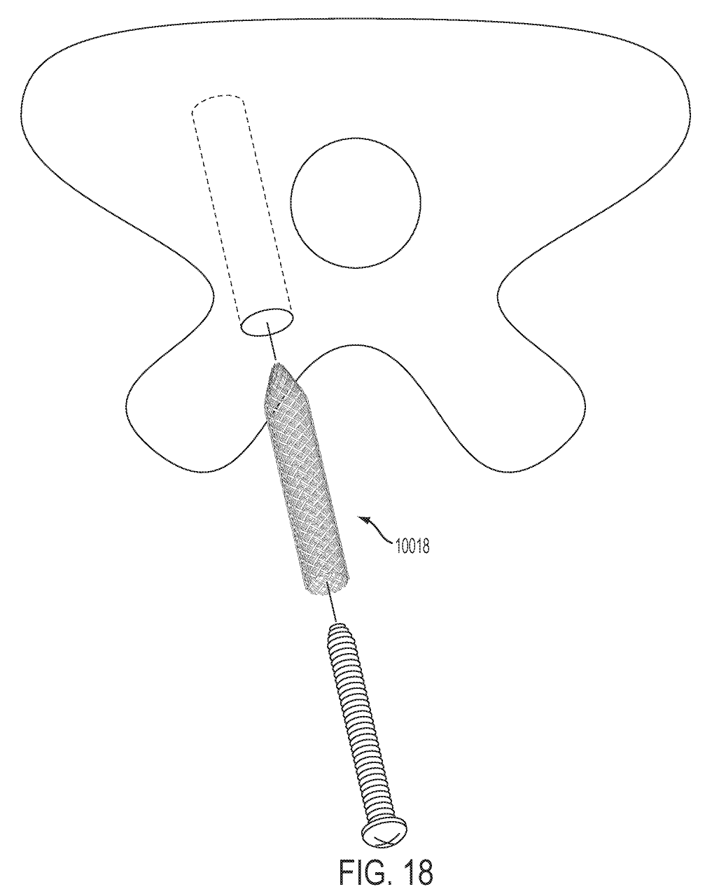

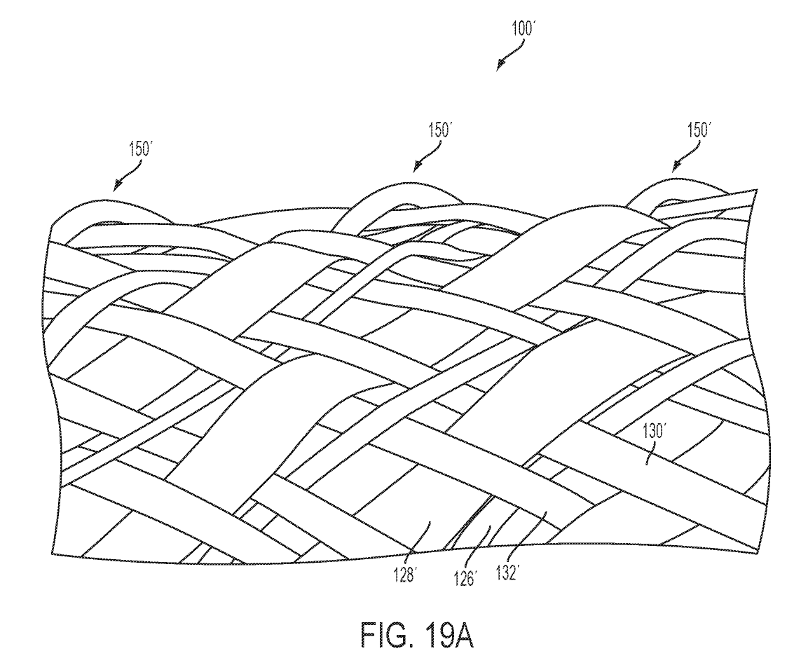

As can be seen from FIG. 18, a screw can be inserted into the woven retention device 10018, which can then be inserted into a hole in a vertebra. Embodiments of the invention are not limited to being used in any particular bone, and may be configured for use in any bone.

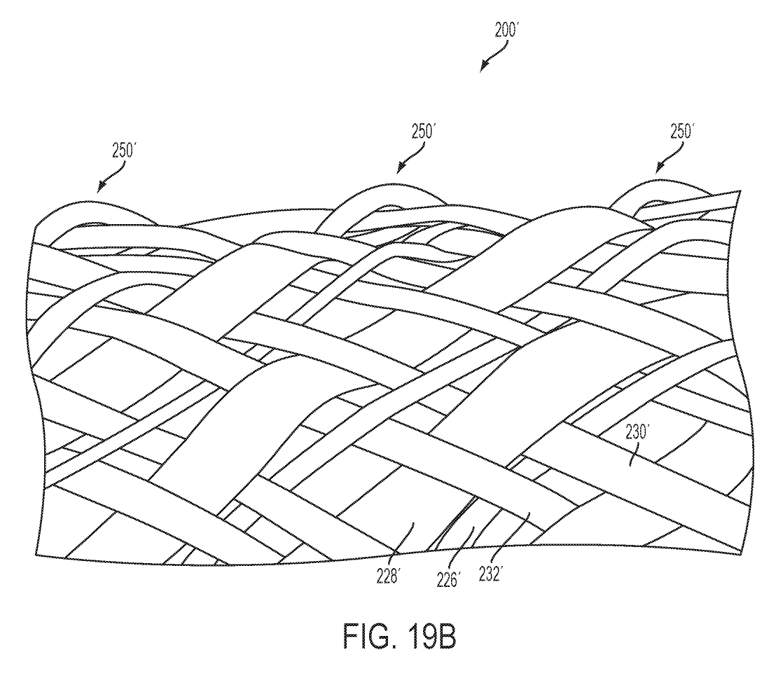

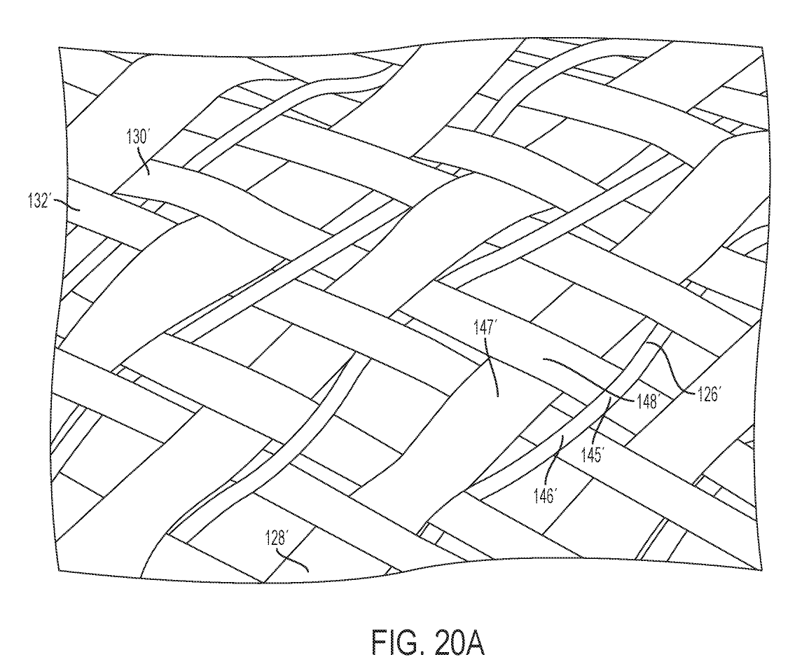

FIG. 19A shows a perspective view of a portion of a woven retention device 100′ according to an embodiment of the present invention. The profile of protuberances 150′ are shown. According to this embodiment, the woven retention device 100′ comprises a pair of monofilaments 126′ and 132′, and a pair of multifilaments 128′ and 130′. In this example, each of the multifilaments 128′ and 130′ has a different width or linear mass thickness. Additionally, each of the monofilaments 126′ and 132′ have a different thickness. Specifically, multifilament 128′ is wider than multifilament 130′, and monofilament 132′ is thicker than monofilament form 126′. However, the specific arrangement and relative thicknesses of the filaments in FIG. 19 are just examples of an embodiment. It should be understood that the monofilaments 126′ and 132′ may have the same or different relative thicknesses, and likewise for the widths or linear mass densities of multifilaments 128′ and 130′.

FIG. 19B shows a perspective view of a portion of a woven patch 200′ according to an embodiment of the present invention. The profile of protuberances 250′ are shown. According to this embodiment, the woven patch 200′ comprises a pair of monofilaments 226′ and 232′, and a pair of multifilaments 228′ and 230′. In this example, each of the multifilaments 228′ and 230′ has a different width or linear mass thickness. Additionally, each of the monofilaments 226′ and 232′ have a different thickness. Specifically, multifilament 228′ is wider than multifilament 230′, and monofilament 232′ is thicker than monofilament form 226′. However, the specific arrangement and relative thicknesses of the filaments in FIG. 19B are just examples of an embodiment. It should be understood that the monofilaments 226′ and 232′ may have the same or different relative thicknesses, and likewise for the widths or linear mass densities of multifilaments 228′ and 230′.

FIG. 20A shows another view of the embodiment shown in FIG. 19A. The intersections of filaments 126′ 128′, 130′, and 132′ are shown to provide examples of the different types of intersections which can provide a plurality of combinations of filament cross-section geometries according to this embodiment. Specifically, intersection 145′ represents the intersection of a monofilament 126′ and mulifilament 130′. Intersection 146′ is the intersection of monofilament 126′ and monofilament 132′, which would represent the thickest protuberance according to this embodiment. Intersection 147′ is the intersection of monofilament 132′ and multifilament 128′. Finally, intersection 148′ is the intersection of multifilament 130′ and multifilament 128′, which would form the smallest intersection thickness according to this embodiment. In the embodiment of FIG. 20A, the plurality of plurality of filaments include thicker monofilaments (e.g., 132′) wound in a first spiral direction of the woven retention device, and smaller monofilaments (e.g., 126′) wound in a second spiral direction of the woven retention device.

FIG. 20B shows another view of the embodiment shown in FIG. 19B. The intersections of filaments 226′, 228′, 230′, and 232′ are shown to provide examples of the different types of intersections which can provide a plurality of combinations of filament cross-section geometries according to this embodiment. Specifically, intersection 245′ represents the intersection of a monofilament 226′ and multifilament 230′. Intersection 246′ is the intersection of monofilament 226′ and monofilament 232′, which would represent the thickest protuberance according to this embodiment. Intersection 247′ is the intersection of monofilament 232′ and multifilament 228′. Finally, intersection 248′ is the intersection of multifilament 230′ and multifilament 228′, which would form the smallest intersection thickness according to this embodiment. In the embodiment of FIG. 20B, the plurality of plurality of filaments include thicker monofilaments (e.g., 232′) wound in a first spiral direction of the woven patch, and smaller monofilaments (e.g., 226′) wound in a second spiral direction of the woven patch.

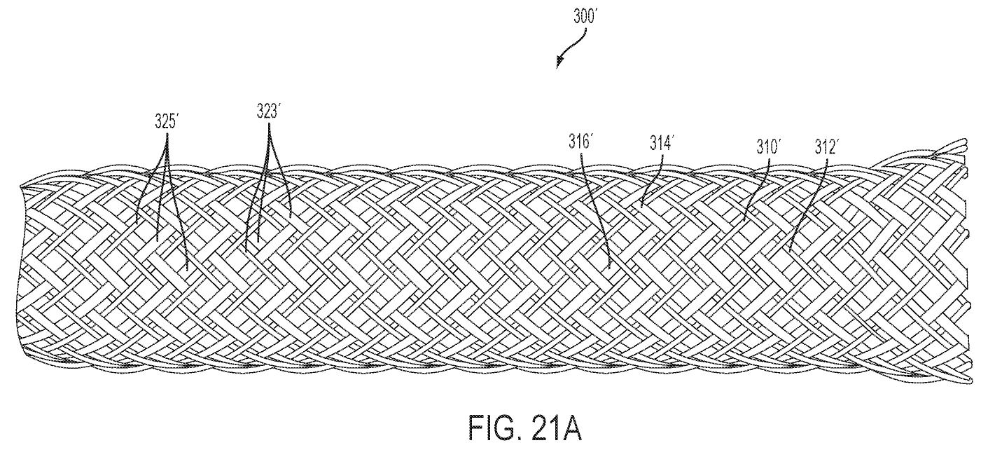

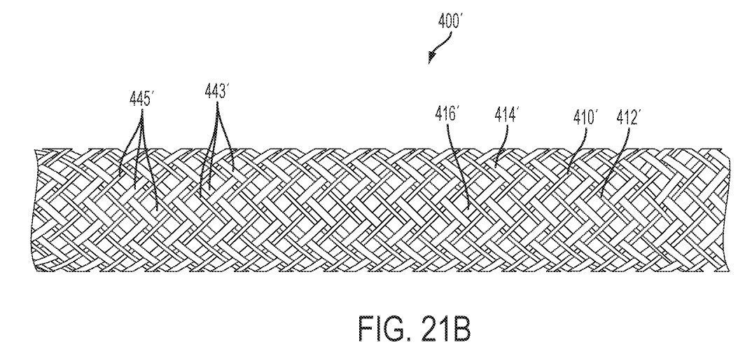

FIG. 21A is a view of an embodiment of the woven retention device 300′ shown in FIGS. 19A and 20A. As shown, the first plurality of filaments 323′, including monofilament 314′ and multifilament 312′, are wound in the first direction, and the second plurality of filaments 125′, including monofilament 310′ and multifilament 316′, are wound in the second direction.

FIG. 21B is a view of an embodiment of the woven patch 400′ shown in FIGS. 19B and 20B. As shown, the first plurality of filaments 423′, including monofilament 414′ and multifilament 412′, are wound in the first direction, and the second plurality of filaments 425′, including monofilament 410′ and multifilament 416′, are wound in the second direction.

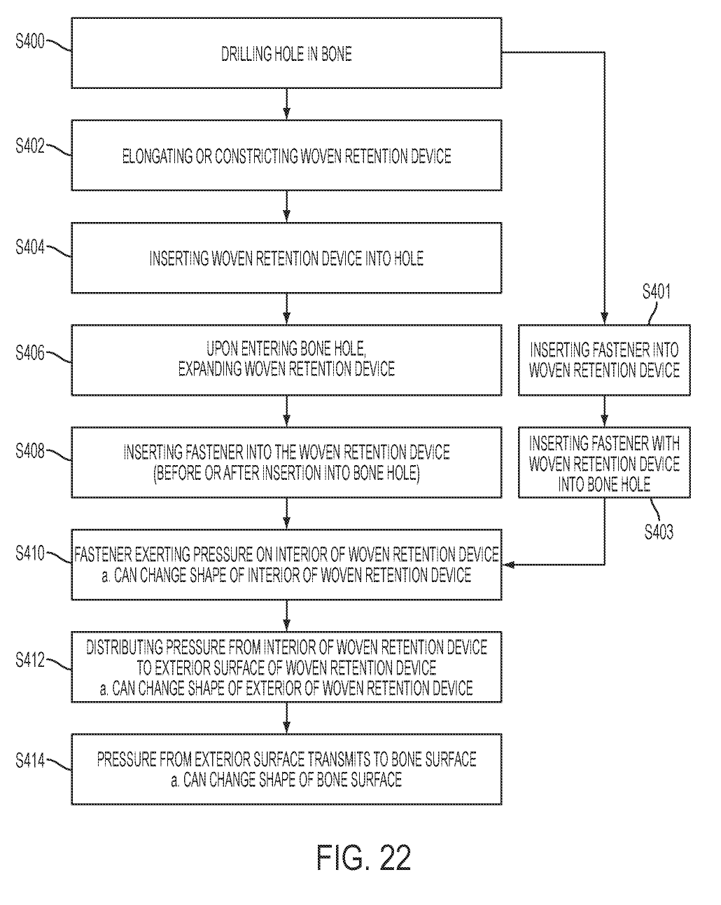

Various methods of using the woven retention device can be used. FIG. 22 details steps that can be performed in conjunction with the woven retention device. The woven retention device may be inserted into a bone hole alone and then a fastener can be inserted. Alternatively, the woven retention device and screw can we combined prior to insertion and the combination inserted into the bone hole. The invention is not limited to the steps described in FIG. 22, is not limited to the order of the steps disclosed, and does not require that certain of the disclosed steps be performed.

In one embodiment, in step S400, a bone can be drilled to form a bone hole. In one embodiment, the woven retention device can be elongated or constricted in step S402, after which in step S404 the woven retention device can be inserted into the bone hole. After step S404, in step S406 the woven retention device upon entering the bone hole can be expanded. Thus, upon entering the bone hole, the woven retention device can expand to a less elongated and constricted state to interface with the bone surface. After step S406, in step S408 the fastener can be inserted into the woven retention device either before or after insertion into the bone hole. Next, the fastener can exert pressure on an interior of the woven retention device in step S410. In step S410, the fastener can optionally change the shape of the interior of the woven retention device. Next, in step S412, pressure from an interior of the woven retention device can be distributed to an exterior surface of the woven retention device. In step S412, the shape of the exterior surface of the woven retention device can optionally change shape. In step S414, pressure from an exterior surface of the woven retention device can transmit to bone surface. In step S414, the pressure transmission to the bone surface can optionally change the shape of the bone surface. In other embodiments, the steps can be performed in different orders or steps can be optionally omitted.

In another embodiment, instead of following steps S402, S404, S406 and S408, in step S401, a fastener can be inserted into the woven retention device before the woven retention device has been inserted into the bone hole, after which in step S403 the fastener with woven retention device can be inserted into the bone hole. After step S403, in step S410 the fastener can optionally change the shape of the interior of the woven retention device. Next, in step S412, pressure from an interior of the woven retention device can be distributed to an exterior surface of the woven retention device. In step S412, the shape of the exterior surface of the woven retention device can optionally change shape. In step S414, pressure from an exterior surface of the woven retention device can transmit to bone surface. In step S414, the pressure transmission to the bone surface can optionally change the shape of the bone surface.

In another embodiment, the distributing pressure step comprises dynamic micro-loading of the woven retention device based on differences in loading patterns of the woven retention device and the interfacing surface shape of the fastener. Based on a uniform radial distribution of the woven retention device, a different loading pattern, or in other words, a dynamic load, is possible. That is, instead of solely pushing or cutting bone, the fastener can deform the woven structure. Further, based on the flexibility of the weave, the woven retention device can facilitate an even distribution of load on uneven bone structure.

In one embodiment, the woven patch can allow for dynamic micro-loading of the woven patch against the bone surface such that the woven patch expands or contracts in thickness based on pressures exerted on the woven patch. In another embodiment, bone cement can be applied to the woven patch to secure the woven patch to the bone. The bone cement can facilitate filling in space between the bone surface and the woven patch, in addition to securing the woven patch to the bone. In an embodiment, the woven patch can serve as a scaffold to encapsulate a fastener.

Thus, a fastener can be inserted into the woven retention device either before or after the woven retention device is inserted into the bone hole. Upon being inserted into the woven retention device, the fastener can exert a pressure on an interior surface of the woven retention device, which can optionally change the shape of the interior surface. The pressure exerted on the interior surface of the woven retention device can distribute pressure to an exterior surface of the woven retention device, which can optionally change the shape of the exterior surface. The change in the exterior surface can allow for better interfacing with the bone surface based on the changes to the exterior surface. The bone surface can optionally change shape based on the pressure that is applied by the woven retention device.

The woven retention device can be beneficial for use with low bone mineral density, which is the amount of mineral matter per square centimeter of bone that is between 1 and 2.5 standard deviations away from young normal adult. Low bone mineral density can include osteoporosis, osteopenia, hyperparathyroidism and/or osteomalacia. A notable part of the woven retention device's interior surface is its ability to engage with the screw without having a matching threaded surface on the interior in a preferred embodiment. The material of the woven retention device can be made of any plastic or fiber. Other materials can also be used, including metallic and natural or biological materials.

The dual interface can be achieved through having a tube-shaped, braided retention device with sufficient rigidity, stability (returning to the woven retention device's original shape or configuration after deformation), and tensile strength when a screw can be inserted to provide sufficient sheer strength to a screw on the one side and a uniform and distributed pressure to the bone on the other side. The woven retention device can have a multi-filament comprising a one-under/one-over arrangement of 45 degree angle intersections and a mono filament that runs adjacent to each of the braids such that each filament goes over two other filaments before going under two filaments (2-over/2-under, twill or herringbone). A three-under/three-over arrangement can also be possible. Other types of weaves are possible (including only a monofilament) as long as there can be sufficient stability, rigidity, compressibility, sheer strength, and/or tensile strength.

The Young's modulus (or load modulus) can also be used to quantify the woven retention device according to some embodiments. In one embodiment, there can be two portions associated with the response of the woven retention device shape upon exertion of pressure from the fastener and upon interfacing with the bony surface. For example, there can be a linear portion to the response curve (stress over strain curve), and there can be a non-linear portion where the material stops behaving elastically. If the material/structure exhibits the linear response over the range of the test (i.e., the amount of stretching performed on the sample), then the sample is “linear.” The amount of stretching performed on the sample is typically an amount of stretching that the sample can be expected to experience in use because all samples will exhibit non-linear response eventually. If the sample exhibits the non-linear response within the test range, the sample can be “non-linear”. In one embodiment, the Young's modulus of the woven retention device can be substantially linear over the load range of the fastener. In another embodiment, the Young's modulus of the woven retention device can be non-linear over the load range.

One configuration of the interlaced filaments can be at a 45 degree braid angle in relation to the axis of the retention device in the position after the woven retention device can be inserted into the hole. Such a braid angle allows for maximum distribution of the protuberances on the exterior surface of the tubular lattice. Other angles are also preferably contemplated to be between 40-50 degree braid angles relative to the retention device longitudinal axis. The woven retention device diameter can be dynamically determined depending on the size of pilot hole diameter such that braid angles are 45 degrees when in hole (which can be less critical for larger screws).

In one embodiment, the woven retention device can be shaped like a hollow rope. In another embodiment, the woven retention device does not require that the filaments be interwoven provided that other characteristics of the filaments provide for a sufficiently rigid and flexible lattice. For example, a retention device for interfacing with a bone surface can include a sleeve body comprising a plurality of intersecting filaments forming a substantially tubular wall, the tubular wall having an interior surface and an exterior surface, the sleeve body being configured to surround at least a portion of a fastener on an interior surface-side of the tubular wall. The retention device can also include a proximal end and a distal end, the sleeve body extending between the proximal and distal ends. The retention device can also include a plurality of protuberances distributed on the tubular wall, each of the plurality of protuberances being formed by intersecting two or more of the plurality of intersecting filaments.

In the retention device, the plurality of intersecting filaments can include a plurality of filament cross-section geometries. Further, the plurality of protuberances can have a plurality of protuberance thicknesses based on a plurality of combinations of the filament cross-section geometries, where a thickness of each of the plurality of protuberances can be based on a particular combination of the plurality of filament cross-section geometries at the intersection point, and the thickness being measured in a radial direction of the sleeve body. In the retention device, the sleeve body, when surrounding at least a portion of the fastener, can be configured to distribute pressure from the fastener on the interior surface-side of a protuberance to an exterior surface-side of two or more protuberances, and the plurality of protuberance thicknesses accommodate deviations in the bone surface. In an alternative configuration, the sleeve body can be configured to distribute pressure from the fastener on the interior surface side of a protuberance to an exterior surface-side of one protuberance having more than one force.

A retention device can include a substantially tubular lattice of intersecting fibers that can be configured to be inserted into a bone tunnel, the tubular lattice including a proximal end and a distal end, the proximal end having a receiving portion that can be configured to receive a fastener along a longitudinal axis of the retention device, wherein: the tubular lattice includes an interior surface that has a distributed interface with protruding and recessed portions that are configured to interact with an exterior surface of the fastener, the tubular lattice includes an exterior surface that has protruding and recessed multiple points of contact configured to interact with an interior bone surface, and the tubular lattice has a degree of stability that maintains a three-dimensional structure of the tubular lattice and has a degree of flexibility, the degree of stability and flexibility allowing for the distributed interface of the interior surface to distribute applied pressure to the protruding and recessed multiple points of contact of the exterior surface, the pressure resulting from the fastener being inserted.

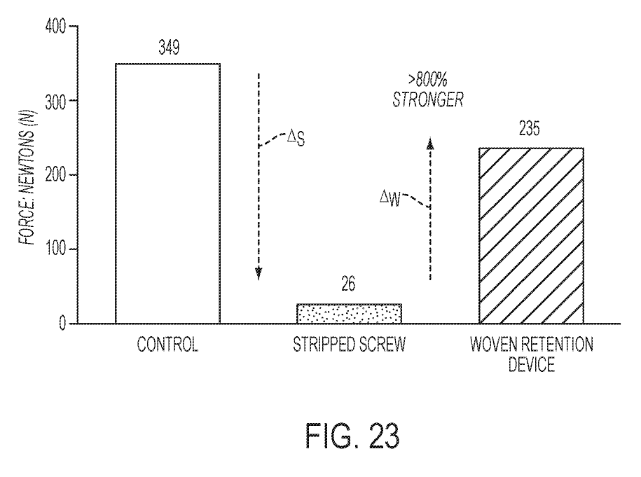

FIG. 23 shows a graph of examples of pullout strengths of a screw in control bone hole, a screw in a stripped bone hole, and, according to an example of an embodiment of the invention, a screw in a woven retention device in a stripped bone hole. A stripped bone hole is one in which a screw, for one reason or another, has lost purchase or fit. For example, the bone may degrade or break to the point that the fit between the bone and the screw is lost, or part of the structure of the bone may be stripped or sheared by the screw itself, for example. As can be seen from FIG. 23, a screw in a stripped bone hole can cause a decrease ΔS in the pullout strength of the screw as compared to a control screw that is in a bone hole that is not stripped. In addition, the woven retention device in accordance with the principles of the invention can cause an increase ΔW in the force required to pullout the screw as compared to the screw by itself in a stripped hole. Although not shown in FIG. 23, the woven retention device can increase the pullout strength of the screw beyond that of a screw in a non-stripped hole, such as the control screw, including in cases where the woven retention device is used in conjunction with a screw in a non-stripped hole.

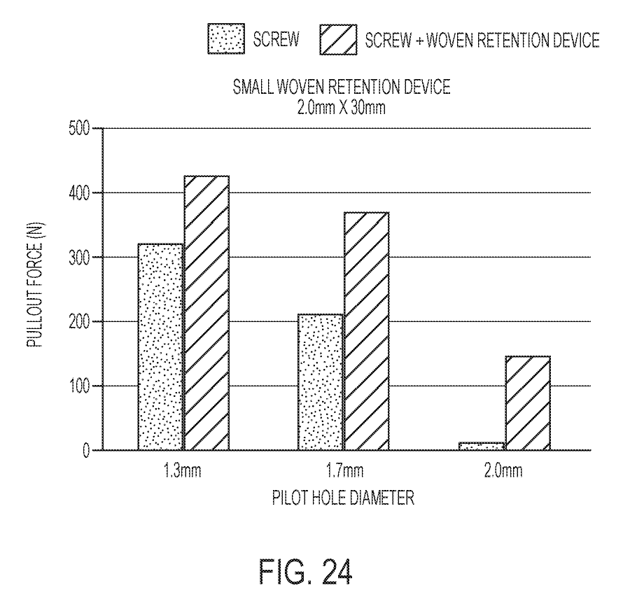

FIG. 24 shows a graph showing examples of different pullout forces between small screws in various different pilot holes. As can be seen from FIG. 24, the combination of the screw and woven fixation device, in accordance with the principles of the invention, has more pullout force in each of the tested sizes.

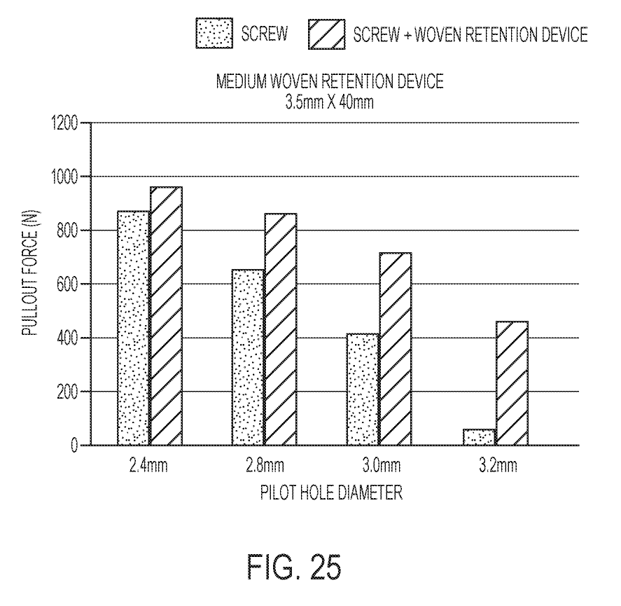

FIG. 25 shows a graph showing examples of different pullout forces between medium screws in various different pilot holes. As can be seen from FIG. 25, the combination of the screw and woven fixation device, in accordance with the principles of the invention, has more pullout force in each of the tested sizes.

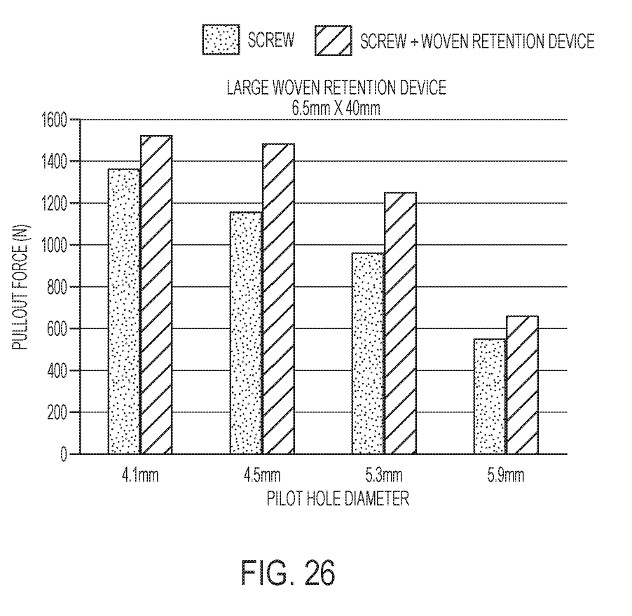

FIG. 26 shows a graph showing examples of different pullout forces between large screws in various different pilot holes. As can be seen from FIG. 26, the combination of the screw and woven fixation device, in accordance with the principles of the invention, has more pullout force in each of the tested sizes.

According to embodiments of the invention, the woven retention device can enhance pullout force percentage compared with a screw alone for a range of hole diameters. However, the woven retention device used with a small screw may allow for a higher percentage increase of pullout force than with medium and large screws. For example, the woven retention device according to an embodiment has been shown to add at least a 10% increase in pullout strength compared with the pullout force of a screw without a woven retention device. Specifically, for small hole diameters, the increase has been shown to be 33% to 77%, according to an example of one embodiment. For medium hole diameters, the increase has been shown to be 10% to 72%, according to another example of an embodiment. Finally, for large hole diameters, the increase has been shown to be 12% to 30% according to another example of an embodiment.

Examples of woven retention devices according to embodiments were fabricated using different combinations of filaments. Table 1 shows details of the five versions of these examples. Each version includes two types of counter clockwise filaments, and two types of clockwise filaments. “Type” refers to whether the filament is mono-filament or multi-filament. “Size” indicates the diameter (measured in millimeters) of the monofilaments, and the linear mass density (measured in decitex, or dtex, which is grams per 10,000 meters) for the multifilament. “# of Carriers” refers to the number of each filament. Version 1 is a combination of mono- and multifilaments. Version 2 is only monofilaments, where the monofilaments are all the same size. Version 3 is a combination of two different sizes of monofilaments. Version 4 is a combination of three different sizes of monofilaments. The woven retention devices in Versions 1-5 each had a braid angle of about 40° to 45°, and were sized to accommodate screw with an inner core diameter of about 6.5 mm (corresponding to the “large” size discussed above). The filaments were made of polyethylene terephthalate (PET).

TABLE 1
Examples of woven retention devices used for
measuring pullout strength

| | Counter Clockwise | | | Clockwise | | |
|---|---|---|---|---|---|---|
| | Filaments | | | Filaments | | |
| | | | # of | | | # of |
| | Type | Size | Carriers | Type | Size | Carriers |
| Version 1 | mono | 0.2 mm | 12 | mono | 0.2 mm | 12 |
| | multi | flat 196 | 12 | multi | flat 196 | 12 |
| | | dtex | | | dtex | |
| Version 2 | mono | 0.2 mm | 12 | mono | 0.2 mm | 12 |
| | mono | 0.2 mm | 12 | mono | 0.2 mm | 12 |
| Version 3 | mono | 0.2 mm | 12 | mono | 0.2 mm | 12 |
| | mono | 0.1 mm | 12 | mono | 0.1 mm | 12 |
| Version 4 | mono | 0.4 mm | 12 | mono | 0.2 mm | 12 |
| | mono | 0.1 mm | 12 | mono | 0.1 mm | 12 |
| Version 5 | mono | 0.2 mm | 12 | mono | 0.2 | 12 |
| | momo | 0.3 mm | 12 | momo | 0.3 | 12 |

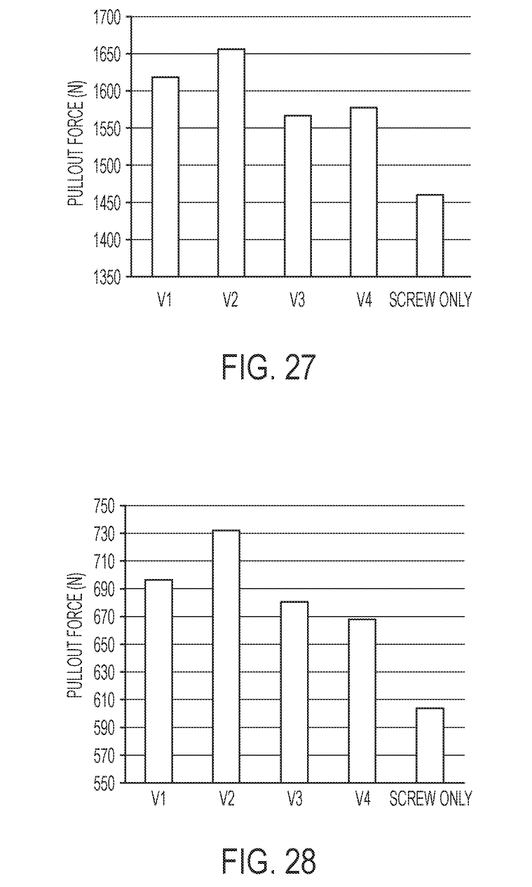

FIGS. 27 and 28 show the results of axial pullout strength using Versions 1-5 of the woven retention device in Table 1 as compared to the pullout strength of a screw without a woven retention device. Both tests used pilot holes with a diameter of 4.1 mm in polyurethane foam, and a screw with an inner core diameter of 6.5 mm and length of 40 mm. In the tests of FIG. 27, 15 pcf rigid polyurethane foam was used. In the tests of FIG. 28, 10 pcf rigid polyurethane foam was used. For all tests, the axial pullout strength was greater when a woven retention device was used, compared to when a screw was used without a woven retention device. Furthermore, FIGS. 27 and 28 show that the greatest pullout strength in these tests was achieved for Version 2, which comprised only monofilaments of the same size. Also, the percentage increase compared to screw-only pullout was greater in the less dense (10 pcf) polyurethane foam, indicating that embodiments of the present invention may well suited for lower density bone, such as osteoporotic or osteopenic bone, for example.

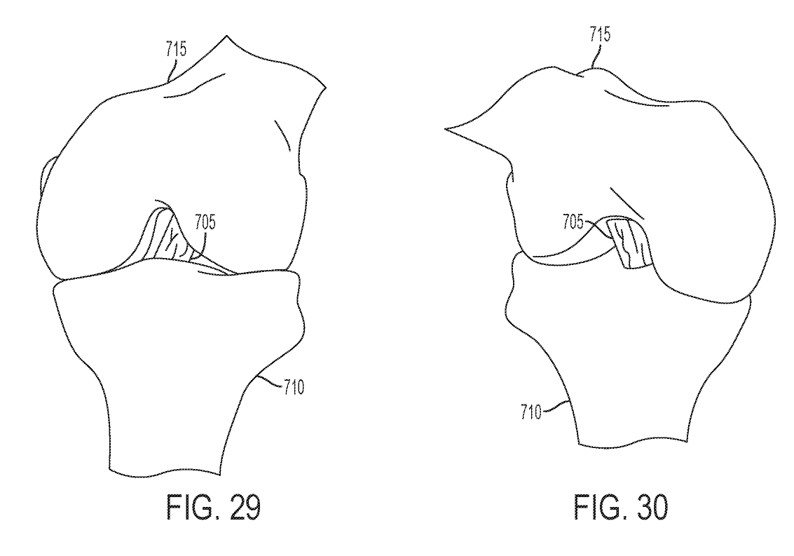

FIGS. 29-37 show embodiments of retention devices, sleeves, lattices and sheaths related to integrating bone and soft tissue. For example, as shown, a lattice 700 for interfacing with a bone surface can include: a sleeve body comprising a plurality of filaments that form a substantially tubular lattice with a plurality of protuberances distributed on an interior surface and an exterior surface of the tubular lattice at a predetermined spatial relationship; a proximal end that is proximal to the sleeve body and that is configured to receive at least one of a fastener and at least a portion of a soft tissue. In an embodiment, the sleeve body can be configured to receive a portion of the soft tissue and not the fastener. In another embodiment, the sleeve body can be configured to receive at least a portion of the fastener and not the soft tissue. In another embodiment, the sleeve body can be configured to receive both the at least a portion of the soft tissue and fastener.

The lattice can further include a distal end that is distal to the sleeve body. The sleeve body can have a plurality of combinations of filament cross-section geometries at intersection points of the interwoven filaments. The plurality of combinations of filament cross-section geometries can form a plurality of different protuberance thicknesses, a thickness of each protuberance being measured in a radial direction of the sleeve body. In an implanted state of the woven retention device, the tubular lattice can be configured to interface with both the soft tissue and the bone surface to secure the soft tissue 705 to the bone surface. The spatial relationship of the protuberances can change according to a function of bone density.

The lattice can further include an anchoring device 711 that is configured to apply pressure to one or more regions of the soft tissue. The sleeve body can distribute the applied pressure through the soft tissue and the bone surface.

The anchoring device 711 can penetrate the soft tissue and protrude into the bone surface. The filaments can be interwoven filaments and the interwoven filaments include at least one set of filament that is a felted filament. The sleeve body can include felted filaments. The sleeve body can include an orthopedic biomaterial, and the sleeve body can be configured to minimize biofilm formation on the bone and/or soft tissue.

FIGS. 29 and 30 shows a first and second view, respectively, of bones 710, 715 with soft tissue 705 that can be repaired or attached, according to embodiments of the present invention. According to various embodiments of the invention, soft tissue can refer, for example, to ligaments, tendons, muscles, facia, fibrous tissues, membranes, other connective tissue, nerves, blood vessels, and other soft tissues, including artificial or transplanted versions or substitutes. One example of soft tissue repair is repairing an anterior cruciate ligament (ACL), for example.

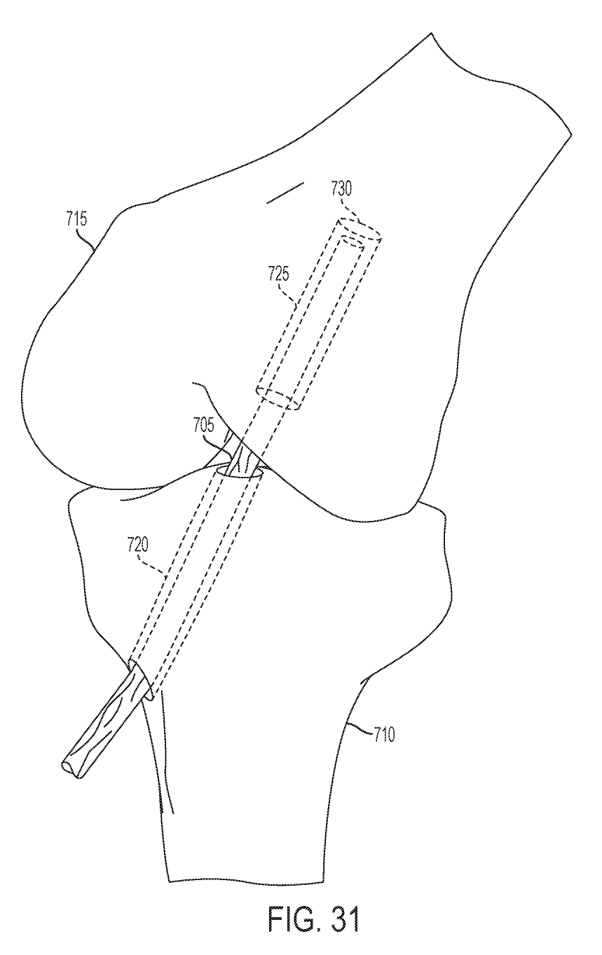

FIG. 31 shows the soft tissue 705 within the bones 710, 715. Bone holes 620 and 725 are shown in the bones 710, 715. According to embodiments, such bone holes can be pre-existing or newly constructed or modified during a surgical procedure. Embodiments of the current invention include devices, systems, and methods for securing soft tissue to bone or within bone holes, such as those shown in FIG. 31.

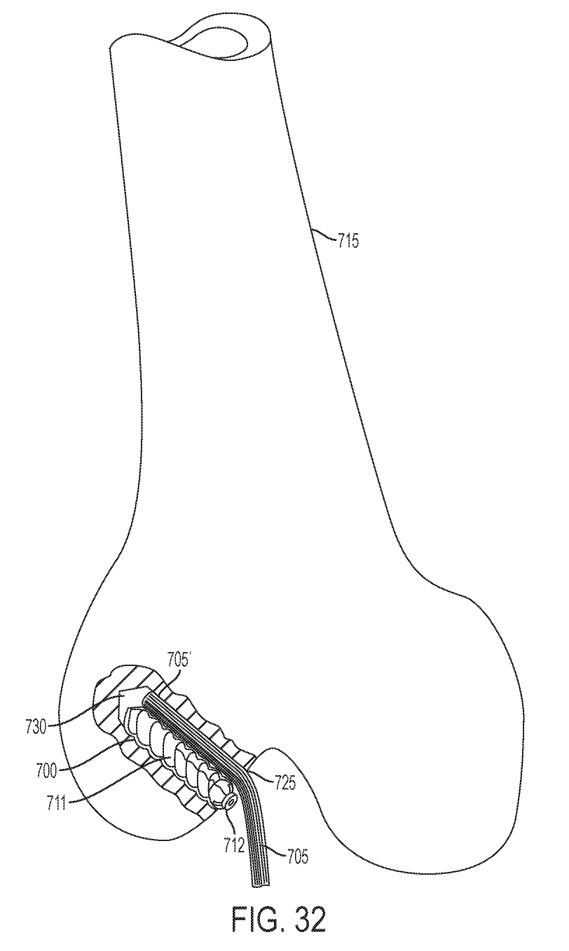

FIG. 32 shows a partial cross-section view of a bone in which a soft tissue 705 is secured according to one embodiment of the present invention. As shown in FIG. 32, the sleeve body can be configured to receive a tendon. A woven retention device 700 is shown in a partial cutaway as it surrounds a fastener 711. The fastener may be, for example, a bone screw. The fastener 711 is placed within the woven retention device 700, and a distal end 705′ of the soft tissue 705 secured within a bone hole 725 between the woven retention device 700 and the wall of the bone hole 725. The proximal end 712 of the fastener 711 may rest at or near the surface of the bone, as shown in FIG. 32. In other embodiments, the fastener 711 may be placed deep within the bone hole 725 such that the proximal end 712 of fastener 711 is within the bone hole 725, as discussed below. In the embodiment shown in FIG. 32, the distal end 730 of the bone hole 725 is closed. However, embodiments are not limited to this configuration, and the distal end of the bone hole may have an opening to a surface of the bone so that, for example, a tack or additional fastener can be used to help secure the soft tissue 705 or woven retention device 700, or both.

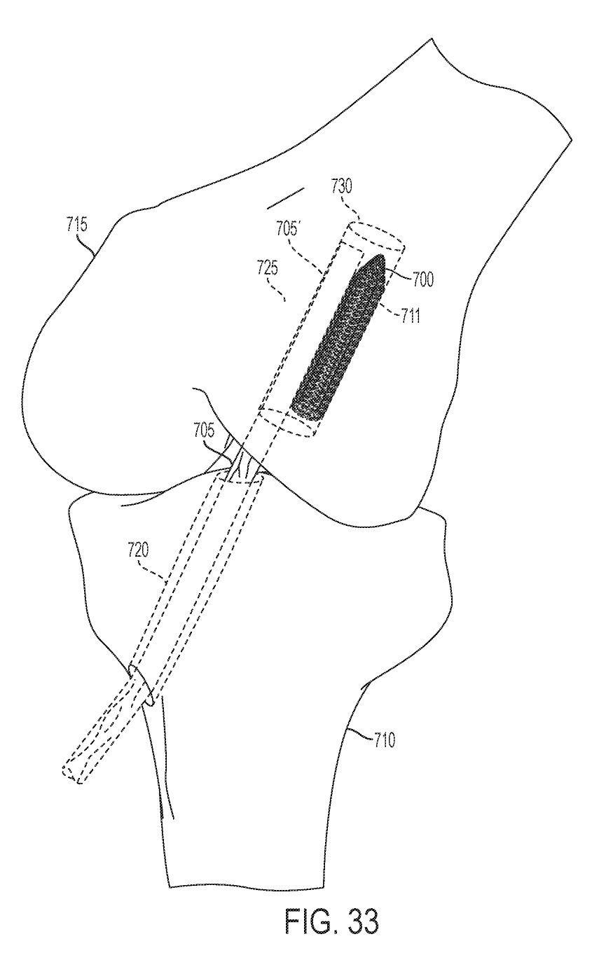

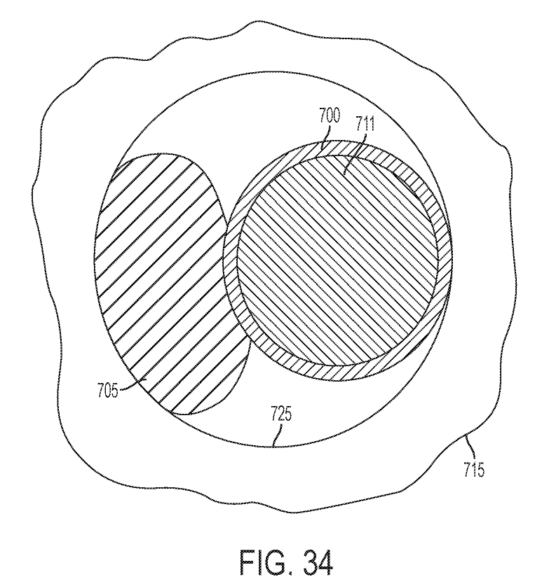

FIG. 33 shows a partial see-through view of a fastener 711 within a woven retention device 700, and a distal end 705′ the soft tissue 705 is secured within a bone hole 725, according to an embodiment. The distal end 705′ may be pinched between the woven retention device 700 and the wall of the bone hole 725. The woven retention device may be the sleeve as shown on FIG. 33 or the patch. According to embodiments of the woven retention device discussed herein, the exterior surface of the woven retention device 700 may have structural characteristics and behavior, including a plurality of protuberances, to engage the wall of the bone hole 725 as well as the distal end 705′ of the bone hole 725. In the embodiment in FIG. 33, pressure may be exerted by the fastener 411 and transmitted by the woven retention device 700 to help secure the soft tissue 705 within the bone. The distal end 705′ of the soft tissue 705 may be held at or near the distal end 730 of the bone hole.

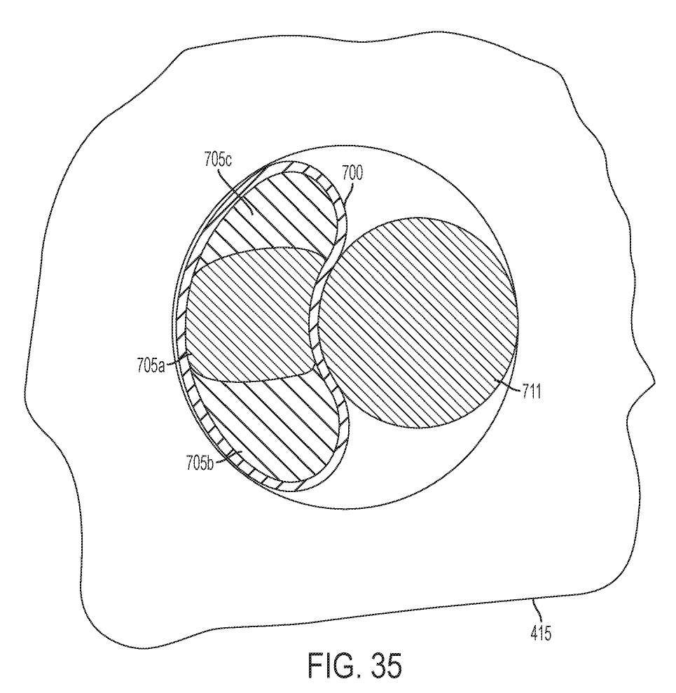

FIG. 34 shows a schematic cross-section view of a fastener 411, woven retention device 700, and soft tissue 705 within a bone hole 725 of bone 715, corresponding to the arrangement shown in FIG. 33.

FIG. 35 shows a schematic cross-section view of a fastener 711, a woven retention device 700, and multiple portions of soft tissue 705a-705c in a bone hole of bone 715, according to an embodiment. The multiple portions of soft tissue 705a-705c can be from different soft tissue, or can be two or more parts of the same piece of soft tissue. In this way, multiple bodies of soft tissue can be secured, or one or more bodies can be twice secured within the woven retention device. Embodiments are not limited to the arrangement shown, and the number of portions of soft tissue can be fewer or more than three. In addition, in this embodiment, the soft tissue 705a-705c is within the woven retention device 700, and the fastener 711 is not within a woven retention device. However, an additional woven retention device may be provided for the screw, or for one or more of the portions of soft tissue 705a-705c. Alternatively, one or more of the portions of soft tissue may be secured on an exterior of the woven retention device 700. According to embodiments of the woven retention device described herein, both an interior surface and an exterior surface of the woven retention devices are able to help secure fasteners or soft tissue within or on a bone.

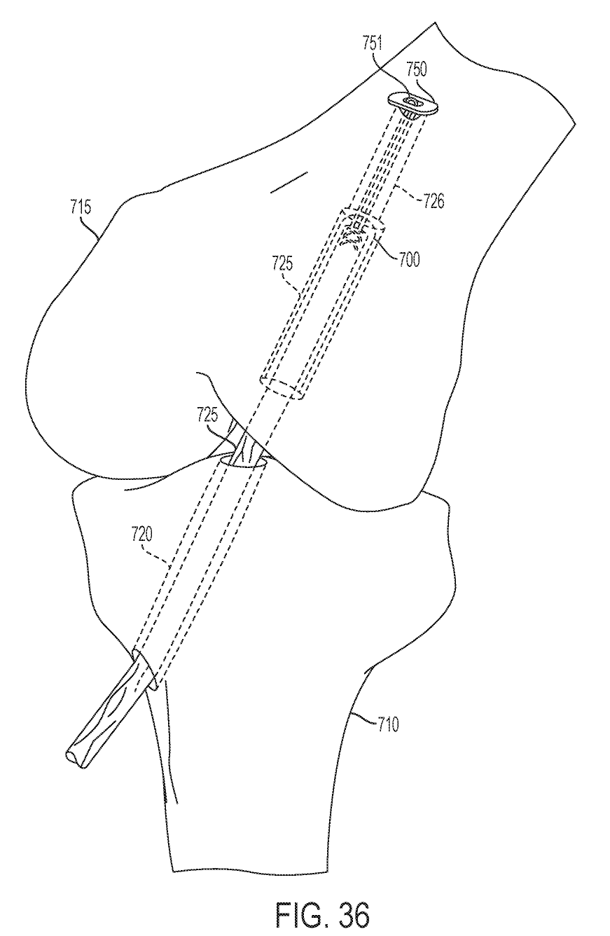

FIG. 36 shows a partial see-through view of a woven retention device 700 and soft tissue 705 within bone holes 720 and 725. In addition, an anchoring device 755 is used according to an embodiment to further secure the soft tissue 705. The anchoring device 755 is placed on an exterior surface of the bone 715 and is attached to the woven retention device 700 and/or soft tissue 705 via a tack or suture 751, for example. The tack or suture 751 can be passed through pores of the woven retention device and optionally can be passed through the soft tissue 705 itself, to help secure the soft tissue and prevent it from falling out of bone hole 725 towards bone 710. By passing the tack or suture 751 through the pores of the woven retention device 700, additional strength and tear resistance is provided by the filaments or material of the woven retention device 700. This can help prevent, for example, tearing of the soft tissue 705.

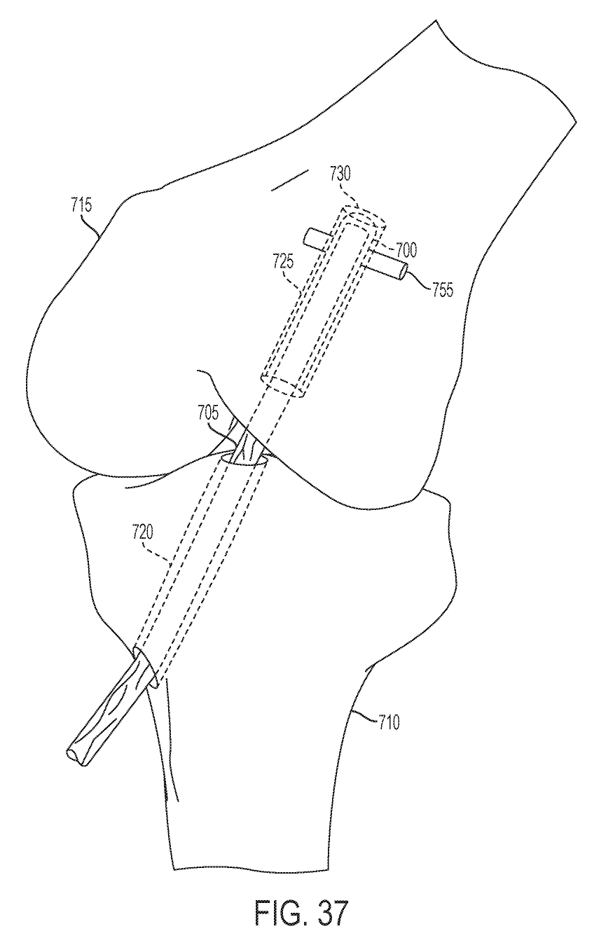

FIG. 37 shows a partial see-through view of a woven retention device 700 and soft tissue 705 within bone holes 720 and 725 and an anchoring device 755 according to an embodiment. The anchoring device 755 according to this embodiment can be a fastener, screw, rod, pin, nail, dowel, tack, suture or other device that is passed through the woven retention device 700 in a direction transverse to a longitudinal axis of the woven retention device 700. A diameter of the anchoring device 755 can be altered to fit through a pore or other opening in the side of the woven retention device 700. Alternatively, the anchoring device 755 may pass near but not through the woven retention device 700, and the woven retention device 700 and/or the soft tissue 705 may be attached to or coupled to the anchoring device 755 to help secure the soft tissue 705. For example, the anchoring device 755 can be connected to the woven retention device 700 or soft tissue 705 using suture or some other connection mechanism.

In another embodiment, the method and apparatus for creating the retention device can be performed by 3D printing to create a porous structure that has raised points of multiple contacts on the inside and the outside. The 3D printing technique can also be used to create a weave or braid structure. A computer readable storage medium having data stored thereon can be executed by a computer processor to cause a computer to perform certain steps that result in a three-dimensional retention device structure.

With 3D printing, it is possible to orient individual polymer molecules or polymer particles such that if one thinks of fibers, just one long continuous polymer material, a relationship between one fiber and another can be separated without the fibers fusing together. Thus, a resulting structure is possible that functions just like a woven retention device. The files used in the 3D printing process may use sophisticated arithmetic in terms of how the polymer material is set up. For example, a process of felting may include taking fibers and inter-relating fibers to create a fabric. The felting process can be based off of nitinol technology such as long continuous fibers (e.g., wires) of nitinol in a generally mesh-like structure that possesses a lattice of fibers with a variety of apertures. There are other means and methods to approach this that are not necessarily woven or braided that may result in a similar mesh-like configuration. In a process similar to felting, another embodiment includes taking small fibers and turning them into a matrix and then putting other fibers together not in a woven pattern, but in a random way that could end up creating a variety of protuberances. Thus, 3D printing can be used as a basis of producing embodiments that disclose multi-dimensional protuberances.

In some embodiments, 3D printing can be used in varying the thickness of the fiber along the length of the fiber so that very specific protuberances, geometries, or distributions of protuberances are possible. Thus, rather than have one thick fiber and a smaller fiber that have a uniform thickness across the length of the device, a fiber that is thick in one portion of the fiber or along multiple portions of that fiber makes it possible to vary wall thickness along the length, and vary localized protuberance thicknesses to create very specific geometries and mechanical performance.

In some embodiments, a non-transitory computer-readable storage medium can have data thereon representing a three-dimensional model suitable for use in manufacturing a three-dimensional retention device 100 for interfacing with a bone surface 104. The non-transitory computer-readable storage medium, when executed by at least one processor, can cause a computing system to use the data in forming the three-dimensional retention device to create a plurality of filaments having input regions that interlace with other filaments. The retention device can include: a sleeve body comprising a plurality of filaments forming a substantially tubular lattice with a plurality of protuberances distributed on an interior surface and an exterior surface of the tubular lattice at a predetermined spatial relationship. The sleeve body can be configured to surround at least a portion of a fastener. Each of the plurality of protuberances can be formed by an intersection point of two or more of the plurality of filaments. The sleeve body can include an orthopedic biomaterial. The woven retention device can include a proximal end that is proximal to the sleeve body and that is configured to receive at least a portion of the fastener; and a distal end that is distal to the sleeve body.

In a first state, the sleeve body can have a plurality of combinations of filament cross-section geometries at the intersection points. The plurality of combinations of filament cross-section geometries can form a plurality of protuberance thicknesses. A thickness of each protuberance can be measured in a radial direction of the sleeve body. In a second state when a fastener is inserted into the tubular lattice, pressure from the fastener can be transmitted to the tubular lattice such that the spatial relationship of the protuberances changes according to a function of bone density and according to a function of an interfacing surface shape of the fastener.

In some embodiments, the woven retention device 100 can include an orthopedic biomaterial that stimulates bone growth. Biomaterials in orthopedics can be made of biocompatible, biofunctional, non-toxic, machinable, moldable, extrudable, having tensile strength, yield strength, elastic modulus, corrosion and fatigue resistance, surface finish, creep, hardness. See Patel and Gohil, “A Review on Biomaterials: Scope, Applications & Human Anatomy Significance,” International Journal of Emerging Technology and Advanced Engineering 2(4): pp. 91-93 (April 2012) (Patel), the content of which is hereby incorporated herein by reference in its entirety. For example, the interwoven filaments can include biomaterials and the biomaterials can be manufactured in fiber format. The woven or non-woven structures described above can be made of non-resorbable or bioabsorbable polymers, metals, biological products or ceramics. Bio resorbable polymer material can be used. For example, the sleeve material can be bioabsorbable and dissolve for complete healing, reduced risk of particulate debris, and have no removal complications as a result. The bioabsorbable polymer can include at least one of thermoplastic aliphatic polyester (PLA), polyglycolide (PGA), polylactide (PLLA) and resorbable polyamides. Alternatively, the sleeve material does not degrade but stays as a structural support of the bone. A non-resolvable polymer material can be biologically suited for use in bone, such as PET (polyehthylene terephthalate), ultra high molecular weight polyethylene, polyether etherketone (PEEK), polyether ketoneketone (PEKK), polypropylene, polyamides, PTFE, calcium phosphate and variations of sutures.

A hydrophilic biomaterial such as a metal can be hydrophilic and attract bone. In one embodiment, metals can also be used such as titanium, tantalum, nickel titanium (nitinol), platinum, cobalt chrome/cobalt chromium, or a blend of all the listed metals. For example, the metals can include at least one of nickel-titanium (Ni—Ti) or nitinol, stainless steel, platinum, titanium, cobalt chrome, cobalt chromium, or any combination thereof. In an embodiment, the metal material can be roughened to create a roughness characteristic that attracts bone, or encourage bone to grow to it or group to it. In an embodiment, the biomaterial such as a metal can have a radioactive property such that the biomaterial can be detected using electromagnetic radiation, such as X-rays. In one embodiment, the woven retention device can be made of fibers of a bone-promoting biomaterial in combination with fibers of a material that does not promote bone growth. For example, the woven retention device can be made of fibers of titanium, which promotes bone growth, as well as PEEK, which promotes bone growth less. Additionally, fibers of PEKK, which can promote bone growth, can be used in combination with titanium and PEEK. In one embodiment, the filaments can include porous fibers.

In an embodiment, the woven retention device can be constructed with an interior surface having a tap of the metallic biomaterial that follows the path of a fastener such as a screw. In such a configuration, the woven retention device is self-tapped to receive an insert, and as the screw follows the path, the woven retention device is configured to expand. In one embodiment, the self-tapping can be produced through the weaving pattern of the fibers or through a mechanical inscribing process that machines thread that matches to the material into which the woven retention device is being inserted. For example, one metal fiber can be included among all other plastic fibers and based on the pitch of the screw, the metal fiber can be designed to follow the tap of the screw.

Biological materials or biologics, such as silk, collagen, and cat gut suture can be used. See Park and Lakes, “Biomaterials: An Introduction,” 1992, Chapter 4 (Park), the content of which is hereby incorporated by reference herein in its entirety. The biological products can include at least one of silk and collagen. Thus, the sleeve can be made of sheet fabric materials such as Silk or Felt that is not woven, but could be created by using collagen. An interior surface could be configured to interface with different structures besides a screw (clamp, smooth, roughened) to provide a strong connection as long as there are many points of contact to provide sufficient sheer strength and a monolithic structure (that is, if one point fails, whole structure does not fail).

In some embodiments, ceramic materials can be used (or bioceramics), which are inert, strong in compression, and biocompatible, such as aluminum oxide, calcium phosphate (hydroxyapatite). See Patel, page 96. For example, the sleeve material can be made of bioactive glass ceramics. See Park chapter 3. The sleeve materials can have bone regenerative qualities (bony apposition).

In some embodiments, the woven retention device 100 can include sleeve materials applied to the woven retention device 100. For example, structural integrity of the woven retention device can be a non-resorbable material, for example PET or PEEK fiber. And the woven retention device is then interlaced with a biologic fiber, such as collagen, to attract or stimulate the bone. Thus, in an embodiment, biological fibers need not provide structural or fixation support, but instead locally stimulate bone formation. Alternatively, the woven retention device can include can include all synthetic materials as non-resorbable materials and instead of interlacing biological fibers, the woven retention device can be coated with an osteostimulative agent.

Instead of a flat fiber, a rougher or pillowy surface is also possible. In an embodiment with a monofilament/multifilament, a textured fiber instead of a flat multifilament could absorb or wick up more biological agent. In one embodiment, the woven retention device can be biofriendly and have a wicking characteristic to absorb plasma-rich platelets. As an example, a method of treating cancer or stimulating bone growth can include as a first step manufacturing a retention device having from plastic or from a biomaterial. The second step can include taking a patient's own blood, spinning it, supercharging it, extracting platelet-rich-plasma, and putting the blood back into the patient. The third step can include dipping the manufactured retention device into the extracted platelet-rich-plasma to coat the retention device. A next step can include inserting the coated retention device into the patient.

Alternatively, or additionally, the manufactured retention device can be inserted into the patient before coating of at least one application of platelet-rich-plasma, and after insertion, the retention device can be injected with the platelet-rich-plasma and the retention device wicks up the platelet-rich-plasma. The step of coating the retention device can take place in the operation room by a professional. In one embodiment, the fibers that work well with the platelet-rich-plasma can be bio friendly and have a wicking characteristic to absorb the platelet-rich-plasma. Thus, in one embodiment, a unique fiber or conventional fiber that is texturized multifilament type of device or a combination of different fibers allow for wicking. Alternatively, an agent could come prepackaged with the retention device, e.g., that is dipped in something before it is inserted and soaked up. Alternatively, the agent could be applied internally and the retention device can wick the agent in situ.

In some embodiments, a combination of natural and synthetic fibers can be used in the manufacturing process of the retention device. For example, a structural piece can be used along with an agent either to stimulate bone to release some kind of therapeutic, to combat an infection, prevent an infection, act as a pain medication, act as a stimulant for bone growth. The stimulant for bone growth can be naturally-occurring, patient's own blood or synthetic, like a hormone.

The sleeve materials can have antibiotic or anti-microbial properties to reduce infections and enhance effectiveness. The woven retention device can be made of a metal that slowly releases ions over time that has anti-microbial properties. Some materials alone have antimicrobial properties. Naturally occurring Silver, for example, can be used as an anti-microbial agent. Thus, impregnating Silver with PEEK can provide an anti-microbial property. Other materials, including metals and polymers, can have anti-microbial properties in combination with other materials. The sleeve material can have drug eluting properties to stimulate bone growth and improve recovery time. And it can be provided at the local level instead of at the systemic level. The sleeve material can also be bioconductive meaning that an allograph fixation sleeve can be made using allographic tissue to create a bone based fixation sleeve in combination with long fiber bone tissue processed by Osteotech, now owned by Medtronic. The allographic tissue can be made out of different material (human based material).

Biofilm

There is a need for devices, systems and methods that enhance the surface of a bone hole to provide enhanced fixation of a bone anchor to the bone. Additionally, there is a need for devices, systems and methods for repairing the surface of the bone hole following damage to the bone hole as in the case of stripping of the hole in the bone when a bone screw is over-tightened. Also, there is a need for devices, systems and methods for providing an enhanced bone hole surface for the reattachment of tendons in, for example anterior/posterior cruciate ligament repair procedures, rotator cuff repair procedures, etc. There is a need for a device that enhances the surface of a bone hole to enhance fixation of a bone anchor to bone and permits bone ingrowth into its structure. There is a need for a single device that enhances the surface of a bone hole to enhance fixation of a bone anchor to bone and accommodates variations in the diameter and depth of the bone hole. Further, there is a need for such devices that have enhanced biocompatibility to aid in tissue and bone healing, regeneration, and growth.

According to an embodiment of the present invention, a retention device for interfacing with a bone surface and impeding biofilm development is provided. The retention device includes a sleeve body including a plurality of filaments forming a substantially tubular lattice with a plurality of protuberances distributed on an interior surface and an exterior surface of the tubular lattice at a predetermined spatial relationship. The sleeve body can surround at least a portion of a fastener, and each of the plurality of protuberances may be formed by an intersection point of two or more of the plurality of filaments that outline a plurality of apertures. The filaments can include an orthopedic biomaterial. The retention device also may include a proximal end that is proximal to the sleeve body and that can receive at least a portion of the fastener, and a distal end that is distal to the sleeve body. In a first state, the sleeve body may have a plurality of combinations of filament cross-section geometries at the intersection points, the plurality of combinations of filament cross-section geometries forming a plurality of protuberance thicknesses. A thickness of each protuberance is measured in a radial direction of the sleeve body. In a second state when a fastener is inserted into the tubular lattice, pressure from the fastener can be transmitted to the tubular lattice such that the spatial relationship of the protuberances changes according to a function of bone density and according to a function of an interfacing surface shape of the fastener.

In an aspect of an embodiment, the retention device can be a woven retention device and the filaments may be interwoven. The orthopedic biomaterial can include a hydrophilic material that attracts bone growth, and the hydrophilic material can be a metal. The orthopedic biomaterial can include non-resorbable polymer fibers. The orthopedic biomaterial can include at least one of osteostimulative, antimicrobial, and plasma-rich-platelet (PRP) agents applied to the filaments. In an embodiment, the non-resorbable polymer fibers are roughened to wick one of osteostimulative, antimicrobial and plasma-rich-platelet agents. The orthopedic biomaterial may include biologic fibers that are configured to absorb into a body, and the non-resorbable polymer fiber and the biologic fibers may be interwoven.

In an embodiment, the interior surface of the retention device can be tapped and can allow for substantially uniformly expanding the sleeve body as the fastener is inserted. In an aspect of an embodiment, the retention device is a non-woven retention device and wherein the sleeve body comprises at least one of silk, felt and collagen.

In an embodiment, the interwoven filaments can include a first plurality of monofilaments that runs in a first helical direction and a second plurality of monofilaments that runs in a direction intersecting the first plurality of monofilaments. For each set of the first and second plurality of monofilaments, there may be a substantially same arrangement of cross-section geometries at every other intersection along that set, the substantially same arrangement being different from an arrangement of cross-section geometries at remaining intersections along that set.

In an embodiment, the retention device may further include a first plurality of multifilaments that runs in the first helical direction and a second plurality of multifilaments that runs in the second direction, the first plurality of monofilaments and the first plurality of multifilaments forming a first plurality of sets of filaments and the second plurality of monofilaments and the second plurality of multifilaments forming a second plurality of sets of filaments. Each of the first plurality of sets of filaments can include a first outer filament and a first inner filament, and each of the second plurality of sets of filaments can include a second outer filament and a second inner filament. The first plurality of monofilaments may be thinner in diameter than the second plurality of monofilaments. The interwoven filaments may follow a two-under/two-over configuration, where at each intersection, the second plurality of monofilaments either overlies both of the intersecting monofilaments or is overlain by both of the intersecting monofilaments and the second plurality of monofilaments overlies one of the intersecting filaments and is overlain by the other of the intersecting filaments.

In an aspect of an embodiment, the first plurality of monofilaments may have a diameter in a range of about 0.1 mm-0.4 mm. The first plurality of monofilaments may have a diameter of 0.2 mm.

The braid angle of the filaments can intersect at approximately 45 degrees. In an embodiment, the orthopedic biomaterial may include a bioresorbable polymer that is configured to at least partially dissolve or be absorbed in the body.

In an aspect of an embodiment, the plurality of apertures of the retention device includes a plurality of differently shaped apertures.

Screw-Activated

In one aspect, an implantable retention device for interfacing with a bone tissue and a fastener can include a hollow tube structure having a proximal end, a distal end, and a body that extends between the proximal and distal ends. The body can have porosity arranged to allow for bone ingrowth from the bone tissue. The body can include an interior surface configured to interface with the fastener and the interior surface can define a first inner diameter in a resting state of the hollow tube structure. The body can include an exterior surface configured to interface with the bone tissue. The hollow tube structure can be configured to uniformly radially compress along at least a portion of the body into a constricted state, and the interior surface can be configured to define a second inner diameter in the constricted state that is smaller than the first inner diameter. The hollow tube structure can be configured to uniformly radially expand along at least a portion of the body into an expanded state, and the interior surface can define a third inner diameter in the expanded state that is larger than the first inner diameter. The implantable retention device can have a degree of flexibility and stability such that, in the constricted state and in the expanded state, the body is biased to return to the resting state.

The implantable retention device can have a shear strength along an axis substantially orthogonal to a longitudinal axis of the hollow tube structure such that the hollow tube structure resists shearing from surface areas of the bone tissue and the fastener up to a predetermined threshold when the fastener is inserted into the bone hole.

The degree of flexibility of the implantable retention device can allow for the hollow tube structure to change to a contorted state when the fastener is inserted. The contorted state can be different than the resting state, the constricted state and the expanded state. In the contorted state, at least portions of the body can interdigitate with variations in a surface of the fastener. When implanted into the bone hole and in the contorted state, at least portions of the body can interdigitate with the bone tissue.

The body can further include a plurality of engagement sites on the exterior and interior surfaces of the body that interact with a surface of the bone tissue and a surface of the fastener, respectively. The plurality of engagement sites can exert a plurality of retaining forces on the surface of the bone and the surface of the fastener to resist removal of the fastener from the implantable retention device and to resist removal of the implantable retention device from the bone. The implantable retention device can be configured such that when an engagement site fails to exert a sufficient retaining force, others of the plurality of points of contact compensate for the failed engagement site. The implantable retention device can be monolithic. The engagement sites can include elements that are raised relative to the exterior and interior surfaces of the body.

A tensile force applied to the hollow tube structure parallel to a longitudinal axis of the hollow tube structure can cause the hollow tube structure to radially constrict and the compressive force applied to the hollow tube structure parallel to the longitudinal axis of the hollow tube structure can cause the hollow tube structure to expand.

A torque in a first direction about the longitudinal axis of the hollow tube body can cause the hollow tube body to radially constrict.

The implantable retention device can further include a screw-activated device including i) a screw having a threaded portion and ii) a bolt that is configured to be threaded along the threaded portion of the screw. A first end of the screw can be attached to the distal end of the implantable retention device and at least a portion of the threaded portion runs along a longitudinal direction of the body inside the implantable retention device. A second end of the screw can be configured to accept the bolt such that when the bolt is moved inside the hollow tube structure, a compressive force is exerted on the implantable retention device by the bolt in a direction parallel to a longitudinal axis of the hollow tube structure. The compressive force radially can expand the hollow tube structure to the expanded state. When the fastener is inserted a predetermined distance into the hollow tube structure, the proximal end of the implantable retention device can be configured to detach from the fastener.

The retention device can be made of at least one of silk, non-woven felt, and collagen.

The fastener can be a screw. The fastener can include a smooth or roughened clamp. The distal end can be tapered.

In another aspect, an implantable retention device for interfacing with a bone tissue and a fastener can include a hollow tube structure having a proximal end, a distal end, and a body that extends between the proximal and distal ends. The body can have porosity arranged to allow for bone ingrowth from the bone tissue. The body can include an interior surface configured to interface with the fastener, where the interior surface can define a first inner diameter in a resting state of the hollow tube structure. The body can further include an exterior surface configured to interface with the bone tissue. The hollow tube structure can be configured to uniformly radially compress along at least a portion of the body into a constricted state, and the interior surface can define a second inner diameter in the constricted state that is smaller than the first inner diameter. The hollow tube structure can be configured to uniformly radially expand along at least a portion of the body into an expanded state, and the interior surface can define a third inner diameter in the expanded state that is larger than the first inner diameter. The implantable retention device can have a degree of flexibility and stability such that, in the constricted state and in the expanded state, the body is biased to return to the resting state.

Soft Tissue

According to an embodiment of the invention, a woven retention device for securing soft tissue relative a bone surface is provided. The woven retention system may include a woven retention device that includes a sleeve body, a proximal end, and a distal end. The sleeve body includes a plurality of interwoven filaments that form a substantially tubular lattice with a plurality of protuberances distributed on an interior surface and an exterior surface of the tubular lattice at a predetermined spatial relationship. The plurality of interwoven filaments may have a plurality of different filament diameters. The proximal end of the woven retention device is proximal to the sleeve body and may be able to receive at least one of a fastener and at least a portion of the soft tissue. The distal end of the woven retention device is distal to the sleeve body. The sleeve body may have a plurality of combinations of filament cross-section geometries at intersection points of the interwoven filaments. The plurality of combinations of filament cross-section geometries can form a plurality of different protuberance thicknesses, a thickness of each protuberance being measured in a radial direction of the sleeve body. In an implanted state of the woven retention device, the tubular lattice can interface with both the soft tissue and the bone surface to secure the soft tissue to the bone surface, and the spatial relationship of the protuberances can change according to a function of bone density.

The woven retention system may further include an anchoring device that can apply pressure to one or more regions of the soft tissue, the sleeve body distributing the applied pressure through the soft tissue and the bone surface. In some embodiments, the anchoring device may penetrate the soft tissue and protrude into the bone surface. The anchoring device can be a tack, a suture, a screw, a bone dowel, or a nail.

According to some embodiments, the woven retention system may further include a fastener that can be implanted into a bone. The soft tissue, sleeve body and the fastener may be extended at least partially through a bone tunnel. The fastener may be a screw having a screw thread and at least one of the exterior surface and the interior surface of the tubular lattice may interact with the screw.

In some embodiments, the plurality of interwoven filaments includes a first plurality of filaments that runs in a first helical direction and a second plurality of filaments that runs in a direction intersecting the first plurality of filaments. Each of the first and second plurality of filaments can include a plurality of sets of filaments, and for each of the sets of filaments, there may be a substantially same arrangement of cross-section geometries at every other intersection along that set, the substantially same arrangement being different from an arrangement of cross-section geometries at remaining intersections along that set. Each set of the plurality of sets of filaments may include at least two filaments of the plurality of interwoven filaments, where the at least two filaments may be substantially parallel to each other and being spaced closer to each other than to filaments in an adjacent set of the plurality of sets of filaments. Each of the sets of filaments may include at least one monofilament. Each of the sets of filaments may also include at least one multifilament. In some embodiments, the first plurality of filaments includes monofilaments having a smaller diameter than a diameter of monofilaments in the second plurality of filaments.

The plurality of interwoven filaments may follow a two-under/two-over configuration, where at each intersection, a first filament of a set of the second plurality of filaments either overlies both filaments of a set of the first plurality of filaments or is overlain by both the filaments of the set of the first plurality of filaments, and a second filament of the set of the second plurality of filaments overlies one of the filaments of the set of the first plurality of filaments and is overlain by another of the filaments of the set of the first plurality of filaments. The plurality of interwoven filaments may include a plurality of flat multifilaments.

The interwoven filaments can outline interstices arranged to allow for bone ingrowth, and the interstices can be differently shaped and differently sized interstices. In a relaxed state, the interwoven filaments may extend around the tubular lattice at an angle in a range of about 40-60 degrees with respect to a longitudinal direction of the woven retention device. In the relaxed state, the interwoven filaments may extend around the tubular lattice at an angle of about 45 degrees with respect to the longitudinal direction of the woven retention device. In some embodiments, the distributed protuberances are arranged in a diamond-shaped pattern grid.

According to some embodiments, the tubular lattice has an outer radius spanning from a furthest outwardly extending protuberance in the radial direction on the exterior surface of the tubular lattice to a center point of the tubular lattice. The tubular lattice may have an inner radius spanning from a furthest inwardly protruding protuberance in the axial direction on the interior surface of the tubular lattice to the center point of the tubular lattice. The tubular lattice may have an average radius that is an average between the outer radius and the inner radius. The outer radius of the tubular lattice is greatest at the intersection point of two filaments of greatest diameter.

In some embodiments, the tubular lattice can transfer pressure applied by the fastener to at least one point on the interior surface to protuberances on the exterior surface adjacent to the at least one point, such that the exterior surface exerts pressure on at least one of bone material and the soft tissue. At least one of the woven retention device and the anchoring device may be made of a resorbable material that resorbs into surrounding tissue over time. The interior surface of the sleeve body can be smoother than the exterior surface of the sleeve body. The interwoven filaments may be made of one or more of silk, collagen, and cat gut suture.

The woven retention system may further include a fastener that can be inserted at least partially within an interior of the tubular lattice through the proximal end. The woven retention device can fix at least a portion of the soft tissue between the exterior surface and the bone surface by transmitting pressure from the fastener on the interior surface to the exterior surface adjacent to the at least a portion of the soft tissue. The fastener may also transmit pressure from the interior surface to the exterior surface adjacent to the bone surface. The fastener may be able to be inserted into a bone hole, and the woven retention device can be used to fix at least a portion of the soft tissue relative to the bone surface by transferring pressure from the fastener on the exterior surface through the soft tissue and portion of the exterior surface adjacent to the bone surface while the at least a portion of the soft tissue is within an interior of the tubular lattice.

According to an embodiment of the invention, a method of anchoring soft tissue using a woven retention system is provided. The method can include providing a sleeve body having a plurality of sets of interwoven filaments that form a substantially tubular lattice with a plurality of protuberances distributed on an interior surface and an exterior surface of the tubular lattice at a predetermined spatial relationship. The plurality of sets of interwoven filaments may have a plurality of different diameters. The method can further include surrounding at least a portion of soft tissue with the sleeve body. The method may also include applying pressure from an anchoring device to the surrounded portion of soft tissue, the woven retention device transmitting the applied pressure from a surface of the woven retention device to a surface of the bone surface of the bone hole according to a function of bone density and according to a function of an interfacing surface shape of the anchoring device, In a first state, the sleeve body may have a plurality of combinations of filament cross-section geometries at the intersection points. The plurality of combinations of filament cross-section geometries can form a plurality of protuberance thicknesses, a thickness of each protuberance being measured in a radial direction of the sleeve body. In a second state when a fastener is inserted into the tubular lattice, pressure from the fastener is transmitted to the tubular lattice such that the spatial relationship of the protuberances may change according to a function of bone density and according to a function of an interfacing surface shape of the fastener. The pressure from the anchoring device changes the spatial relationship of protuberances on the interior surface of the woven retention device, and the pressure from the interior surface changes the spatial relationship of protuberances of the exterior surface of the woven retention device.

In some embodiments, the applying of the pressure includes penetrating the anchoring device through the soft tissue and into the bone surface. The anchoring device can be one of a tack, a suture, a screw, a bone dowel, and a nail. The anchoring device can be made of a resorbable material that resorbs into surrounding tissue over time.

The method may further include providing a fastener to which the anchoring device applies the pressure. The soft tissue, sleeve body and the fastener may be extended at least partially through a bone tunnel. In some embodiments, the interior surface of the sleeve body is smoother than the exterior surface of the sleeve body such that the sleeve body is adapted to interface with the soft tissue and bone surface. The interwoven filaments can be made of one or more of silk, collagen, and cat gut suture.

According to an embodiment of the invention, a method of anchoring soft tissue using a woven retention system is provided. The method includes providing a sleeve body having a plurality of sets of interwoven filaments that form a substantially tubular lattice with a plurality of protuberances distributed on an interior surface and an exterior surface of the tubular lattice at a predetermined spatial relationship. The plurality of sets of interwoven filaments may have a plurality of different diameters. The method may further include inserting the sleeve body and at least a portion of the soft tissue into a bone hole, the portion of the soft tissue being adjacent to the exterior surface of the tubular lattice in the bone hole. The method may also include applying pressure from a fastener inside the sleeve body to the portion of the soft tissue that is adjacent to the exterior surface, the woven retention device transmitting the applied pressure from at least a portion of the exterior surface to a surface of the bone hole according to a function of bone density and according to a function of an interfacing surface shape of the fastener. In a first state, the sleeve body can have a plurality of combinations of filament cross-section geometries at the intersection points. The plurality of combinations of filament cross-section geometries can form a plurality of protuberance thicknesses, a thickness of each protuberance being measured in a radial direction of the sleeve body. In a second state when the fastener is inserted into the tubular lattice, pressure from the fastener is transmitted to the tubular lattice such that the spatial relationship of the protuberances changes according to a function of bone density and according to a function of an interfacing surface shape of the fastener. The pressure from the anchoring device can change the spatial relationship of protuberances on the interior surface of the woven retention device, and the pressure from the interior surface can change the spatial relationship of protuberances of the exterior surface of the woven retention device.

Patch

According to one embodiment of the present invention, a woven patch for interfacing with a bone surface is provided. The patch includes a sleeve body including a plurality of sets of interwoven filaments that form a lattice with a plurality of protuberances distributed on an interior surface and an exterior surface of the lattice at a predetermined spatial relationship, the plurality of sets of interwoven monofilaments having a plurality of different diameters. The sleeve body can surround at least a portion of a fastener. The patch also may include a first end that can interface with at least a portion of the fastener, and a second end that is opposite of the first end to the sleeve body. In a first state, the sleeve body may have a plurality of combinations of filament cross-section geometries at intersection points of the interwoven filaments, the plurality of combinations of filament cross-section geometries forming a plurality of protuberance thicknesses. A thickness of each protuberance may be measured in a direction as a thickness of the sleeve body. In a second state, when a fastener is inserted into or applied to the lattice, pressure from the fastener is transmitted to the lattice such that the spatial relationship of the protuberances changes according to a function of bone density and according to a function of an interfacing surface shape of the fastener.

In an aspect of an embodiment of the present invention, the plurality of sets of interwoven filaments includes a first plurality of monofilaments that runs in a first direction and a second plurality of monofilaments that runs in a direction intersecting the first plurality of monofilaments. For each set of the first and second plurality of monofilaments, there may be a substantially same arrangement of cross-section geometries at every other intersection along that set. The substantially same arrangement can be different from an arrangement of cross-section geometries at remaining intersections along that set. In an embodiment, the first end has a distal tip with a first diameter, and the receiving portion has a second diameter that is greater than the first diameter.

In an aspect of an embodiment, the patch further includes a first plurality of multifilaments that runs in the first direction and a second plurality of multifilaments that runs in the second direction. The first plurality of monofilaments and the first plurality of multifilaments forming a first plurality of sets of filaments and the second plurality of monofilaments and the second plurality of multifilaments forming a second plurality of sets of filaments. Each of the first plurality of sets of filaments can include a first outer filament and a first inner filament, and each of the second plurality of sets of filaments can include a second outer filament and a second inner filament. In an embodiment, the first plurality of monofilaments are thinner in diameter than the second plurality of monofilaments. In a further embodiment, the plurality of interwoven filaments follow a two-under/two-over configuration, where at each intersection, the second plurality of monofilaments either overlies both of the intersecting monofilaments or is overlain by both of the intersecting monofilaments and the second plurality of monofilaments overlies one of the intersecting filaments and is overlain by the other of the intersecting filaments. The first plurality of monofilaments may have a diameter in a range of about 0.1 mm-0.4 mm. The first plurality of monofilaments may have a diameter of 0.2 mm. In an aspect of an embodiment, the plurality of interwoven filaments may include a plurality of flat multifilaments.

In an embodiment, the interwoven filaments outline interstices that allow for bone ingrowth, and the interstices formed by the intersecting filaments include differently shaped and differently sized interstices. The plurality of interwoven filaments may be arranged in a two-under/two-over configuration. In another embodiment, the plurality of interwoven filaments may be arranged in a one-under/one-over configuration. In yet another embodiment, the plurality of interwoven filaments may be arranged in a two-over/one-under configuration. In a further embodiment, the plurality of interwoven filaments may be arranged in a three-under/three-over configuration.

In an aspect of an embodiment, the woven patch can further include the fastener. The fastener can be a screw having a screw thread and the interior surface is configured to interact with the screw. The fastener can apply pressure to the interior surface, the pressure being transmitted to protuberances on the exterior surface adjacent to the protuberance on the exterior surface that interior surface and exerting pressure on bone material.

In an embodiment, the interwoven filaments extend around the lattice at an angle of about 45 degrees with respect to a parallel direction of the woven patch. The distributed protuberances may be arranged in a diamond-shaped pattern grid. The woven patch may have a length in a range from about 30 mm to 40 mm.

In another embodiment of the present invention, a patch for interfacing with a bone surface is provided. The patch includes a lattice of intersecting fibers that can be inserted into a bone tunnel, the lattice including a proximal end and a distal end, the proximal end having a receiving portion that can receive a fastener along a longitudinal axis of the patch. The lattice may include an interior surface that has a distributed interface with protruding and recessed portions that can interact with an exterior surface of the fastener. The lattice may include an exterior surface that has protruding and recessed multiple points of contact configured to interact with an interior bone surface. The lattice can have a degree of stability that maintains a three-dimensional structure of the lattice and has a degree of flexibility, the degree of stability and flexibility allowing for the distributed interface of the interior surface to distribute applied pressure to the protruding and recessed multiple points of contact of the exterior surface.

In an aspect of an embodiment of the present invention, the plurality of intersecting fibers includes sets of a first plurality of monofilaments that runs in a first direction and a second plurality of monofilaments that runs in a direction intersecting the first plurality of monofilaments. For each set of the first and second plurality of monofilaments, there can be a substantially same arrangement of cross-section geometries at every other intersection along that set, the substantially same arrangement being different from an arrangement of cross-section geometries at remaining intersections along that set. The first plurality of monofilaments can be thinner in diameter than the second plurality of monofilaments, in an embodiment. The first plurality of monofilaments can have a diameter in a range of about 0.1 mm-0.4 mm. In an embodiment, the distal end has a distal tip with a first diameter, and the receiving portion has a second diameter that is greater than the first diameter.

In an embodiment, the intersecting fibers may follow a two-under/two-over configuration, where at each intersection, the second plurality of monofilaments either overlies both of the intersecting monofilaments or is overlain by both of the intersecting monofilaments and the second plurality of monofilaments overlies one of the intersecting filaments and is overlain by the other of the intersecting filaments. The intersecting fibers may outline interstices that allow for bone ingrowth, and the interstices formed by the intersecting fibers can include differently shaped and differently sized interstices. In a relaxed state, the intersecting fibers can extend around the lattice at an angle of about 45 degrees with respect to a parallel direction of the woven patch.

In an aspect of an embodiment, when the fastener applies pressure to the interior surface, the pressure is transmitted to protuberances on the exterior surface adjacent to the protuberance on the exterior surface that interior surface and exerting pressure on bone material.

According to another embodiment of the present invention, a method of applying a woven patch is provided. The method includes inserting or applying the woven patch into a bone hole or onto a bone surface. The method also include distributing pressure from a fastener being inserted into or applied onto the woven patch from an interior surface of the woven patch to an exterior surface of the woven patch for transmission of pressure to bone surface of the bone hole according to a function of bone density and according to a function of an interfacing surface shape of the fastener. The pressure from the fastener can change the spatial relationship of protuberances on the interior surface of the woven patch, and the pressure from the interior surface can change the spatial relationship of protuberances of the exterior surface of the woven patch. In an aspect of an embodiment, the fastener may be inserted into the woven patch after the woven patch has been inserted into the bone hole. In an aspect of an embodiment, the fastener may be inserted into the woven patch before the woven patch has been inserted into the bone hole. The pressure transmitted to the bone surface can be adapted to change shape of the bone surface of the bone hole.

In an aspect of an embodiment, the method can further include providing the woven patch. The distributing pressure step can include dynamic micro-loading of the woven patch based on differences in loading patterns of the woven patch and the interfacing surface shape of the fastener. In an embodiment, the method may further include elongating or constricting the woven patch for fitting the woven patch inside the bone hole, and expanding the woven patch upon entering the bone hole.

The applying the woven patch onto the bone surface can include surrounding a substantial portion of the bone with the woven patch, in one aspect of an embodiment. The applying the woven patch onto the bone surface may include applying a bone cement to the woven patch and the bone surface.

The method may further include providing a woven patch according to any of the embodiments discussed herein.

Method of Manufacturing Alternative Materials

According to an embodiment of the invention, a retention device for interfacing with a bone surface is provided. In one embodiment, the retention device is woven. However, in another embodiment, the retention device is non-woven.

According to an embodiment of the present invention, a method of manufacturing a retention device for treating infection, treating cancer, preventing infection or preventing disease is provided. The method includes providing a retention device for interfacing with a bone surface. The retention device may include a sleeve body having a plurality of filaments forming a substantially tubular lattice with a plurality of protuberances distributed on an interior surface and an exterior surface of the tubular lattice at a predetermined spatial relationship. The sleeve body can surround at least a portion of a fastener. Each of the plurality of protuberances can be formed by an intersection point of two or more of the plurality of filaments. The filaments can include an orthopedic biomaterial. The retention device can further include a proximal end that is proximal to the sleeve body and that can receive at least a portion of the fastener. The retention device may further include a distal end that is distal to the sleeve body. According to an embodiment, the method may further include extracting platelet-rich-plasma from a subject, and applying the platelet-rich-plasma to the retention device. The method further includes inserting the applied retention device into a bone hole having the bone surface. According to an embodiment, in a first state, the sleeve body has a plurality of combinations of filament cross-section geometries at the intersection points, the plurality of combinations of filament cross-section geometries forming a plurality of protuberance thicknesses. A thickness of each protuberance is measured in a radial direction of the sleeve body. In a second state when a fastener is inserted into the tubular lattice, pressure from the fastener is transmitted to the tubular lattice such that the spatial relationship of the protuberances changes according to a function of bone density and according to a function of an interfacing surface shape of the fastener.

According to an embodiment, the method further includes inserting a fastener into the inserted retention device. In an aspect of an embodiment, the platelet-rich-plasma can be applied before inserting the retention device into the bone hole. The retention device can be a woven retention device where the filaments are interwoven.

In an aspect of an embodiment, the orthopedic biomaterial can include a hydrophilic material that attracts bone growth. The hydrophilic material can be a metal. In an aspect of an embodiment, the orthopedic biomaterial can include non-resorbable polymer fibers. The orthopedic biomaterial can include at least one of osteostimulative, antimicrobial, and plasma-rich-platelet (PRP) agents applied to the filaments. The non-resorbable polymer fibers can be roughened to wick one of osteostimulative, antimicrobial and plasma-rich-platelet agents. The orthopedic biomaterial can also include biologic fibers that are configured to absorb into a body, where the non-resorbable polymer fiber and the biologic fibers are interwoven. In an aspect of an embodiment, the orthopedic biomaterial includes a bioresorbable polymer that is configured to at least partially dissolve or be absorbed in the body.

In an embodiment, the interior surface of the retention device is tapped and allows for substantially uniformly expanding the sleeve body as the fastener is inserted.

In an embodiment of the present invention, the retention device is a non-woven retention device where the sleeve body includes at least one of silk, felt and collagen.

In an aspect of an embodiment, the interwoven filaments include a first plurality of monofilaments that runs in a first helical direction and a second plurality of monofilaments that runs in a direction intersecting the first plurality of monofilaments. For each set of the first and second plurality of monofilaments, there is a substantially same arrangement of cross-section geometries at every other intersection along that set, the substantially same arrangement being different from an arrangement of cross-section geometries at remaining intersections along that set.

In an embodiment, the method further includes a first plurality of multifilaments that runs in the first helical direction and a second plurality of multifilaments that runs in the second direction, the first plurality of monofilaments and the first plurality of multifilaments forming a first plurality of sets of filaments and the second plurality of monofilaments and the second plurality of multifilaments forming a second plurality of sets of filaments. Each of the first plurality of sets of filaments includes a first outer filament and a first inner filament, and each of the second plurality of sets of filaments includes a second outer filament and a second inner filament. The first plurality of monofilaments can be thinner in diameter than the second plurality of monofilaments. The first plurality of monofilaments can have a diameter in a range of about 0.1 mm-0.4 mm. The first plurality of monofilaments may have a diameter of 0.2 mm.

In an embodiment, the interwoven filaments follow a two-under/two-over configuration, where at each intersection, the second plurality of monofilaments either overlies both of the intersecting monofilaments or is overlain by both of the intersecting monofilaments and the second plurality of monofilaments overlies one of the intersecting filaments and is overlain by the other of the intersecting filaments. The first plurality of monofilaments can have a diameter in a range of about 0.1 mm-0.4 mm. The interwoven filaments can be arranged in a two-under/two-over configuration. The interwoven filaments can be arranged in a one-under/one-over configuration. The interwoven filaments can be arranged in a two-over/one-under configuration. The interwoven filaments can be arranged in a three-under/three-over configuration.

In an embodiment, the distal end has a distal tip with a first diameter, and the receiving portion has a second diameter that is greater than the first diameter. The plurality of filaments can include a plurality of flat multifilaments. The flat multifilaments may be roughened for wicking.

In an aspect of an embodiment, the filaments outline interstices that allow for bone ingrowth, and the interstices formed by the intersecting filaments include differently shaped and differently sized interstices. In an embodiment, the method further includes the fastener. The fastener can be a screw having a screw thread and the interior surface is configured to interact with the screw. The distal end of the device may be closed. In the first state, the interwoven filaments extend around the tubular lattice at an angle of about 45 degrees with respect to a longitudinal direction of the retention device. The distributed protuberances can arranged in a diamond-shaped pattern grid. The tubular lattice may have an outer radius spanning from a furthest outwardly extending protuberance in the radial direction on the exterior surface of the tubular lattice to a center point of the tubular lattice, the tubular lattice having an inner radius spanning from a furthest inwardly protruding protuberance in the axial direction on the interior surface of the tubular lattice to the center point of the tubular lattice, and the tubular lattice having an average radius that is an average between the outer radius and the inner radius. The outer radius of the tubular lattice can be greatest at the intersection point of two of the thick monofilaments. The tubular lattice may have an average diameter that is in a range of about 1.5 mm to 9.0 mm. The retention device may have a length in a range from about 30 mm to 40 mm. When the fastener applies pressure to the interior surface, the pressure is transmitted to protuberances on the exterior surface adjacent to the protuberance on the exterior surface that interior surface and exerting pressure on bone material.

In another embodiment of the present invention, a non-transitory computer-readable storage medium is provided having data thereon representing a three-dimensional model suitable for use in manufacturing a three-dimensional retention device for interfacing with a bone surface. The non-transitory computer-readable storage medium, when executed by at least one processor, can cause a computing system to use the data in forming the three-dimensional retention device to create a plurality of filaments having input regions that interlace with other filaments. The retention device may include a sleeve body including a plurality of filaments forming a substantially tubular lattice with a plurality of protuberances distributed on an interior surface and an exterior surface of the tubular lattice at a predetermined spatial relationship. The sleeve body can surround at least a portion of a fastener. Each of the plurality of protuberances is formed by an intersection point of two or more of the plurality of filaments, and the filaments can include an orthopedic biomaterial. The retention device may further include a proximal end that is proximal to the sleeve body and that is configured to receive at least a portion of the fastener, and a distal end that is distal to the sleeve body. In a first state, the sleeve body has a plurality of combinations of filament cross-section geometries at the intersection points, the plurality of combinations of filament cross-section geometries forming a plurality of protuberance thicknesses, a thickness of each protuberance being measured in a radial direction of the sleeve body. In a second state when a fastener is inserted into the tubular lattice, pressure from the fastener is transmitted to the tubular lattice such that the spatial relationship of the protuberances changes according to a function of bone density and according to a function of an interfacing surface shape of the fastener.

Alternative Materials

According to an embodiment of the invention, a retention device for interfacing with a bone surface is provided. In one embodiment, the retention device is woven. However, in another embodiment, the retention device is non-woven.

A retention device for interfacing with a bone surface can include a sleeve body having a plurality of filaments forming a substantially tubular lattice with a plurality of protuberances distributed on an interior surface and an exterior surface of the tubular lattice at a predetermined spatial relationship. The sleeve body can be configured to surround at least a portion of a fastener, and each of the plurality of protuberances can be formed by an intersection point of two or more of the plurality of filaments. The filaments can include an orthopedic biomaterial. The retention device can include a proximal end that is proximal to the sleeve body and that is configured to receive at least a portion of the fastener. The retention device can include a distal end that is distal to the sleeve body. In a first state, the sleeve body can have a plurality of combinations of filament cross-section geometries at the intersection points, and the plurality of combinations of filament cross-section geometries can form a plurality of protuberance thicknesses. A thickness of each protuberance can be measured in a radial direction of the sleeve body. In a second state when a fastener is inserted into the tubular lattice, pressure from the fastener can be transmitted to the tubular lattice such that the spatial relationship of the protuberances changes according to a function of bone density and according to a function of an interfacing surface shape of the fastener.

The retention device can be a woven retention device and the filaments can be interwoven. The orthopedic biomaterial can include a hydrophilic material that attracts bone growth. The hydrophilic material can be a metal. The interior surface of the retention device can be tapped and can allow for substantially uniformly expanding the sleeve body as the fastener is inserted. The orthopedic biomaterial can include non-resorbable polymer fibers. The orthopedic biomaterial can include at least one of osteostimulative, antimicrobial, and plasma-rich-platelet (PRP) agents applied to the filaments. The non-resorbable polymer fibers are roughened to wick one of osteostimulative, antimicrobial and plasma-rich-platelet agents. The orthopedic biomaterial can include biologic fibers that are configured to absorb into a body, and wherein the non-resorbable polymer fiber and the biologic fibers can be interwoven. The retention device can be a non-woven retention device and the sleeve body can include at least one of silk, felt and collagen.

The interwoven filaments can include a first plurality of monofilaments that runs in a first helical direction and a second plurality of monofilaments that runs in a direction intersecting the first plurality of monofilaments. For each set of the first and second plurality of monofilaments, there can be a substantially same arrangement of cross-section geometries at every other intersection along that set. The substantially same arrangement can be different from an arrangement of cross-section geometries at remaining intersections along that set.

The retention device can further include a first plurality of multifilaments that runs in the first helical direction and a second plurality of multifilaments that runs in the second direction. The first plurality of monofilaments and the first plurality of multifilaments can form a first plurality of sets of filaments and the second plurality of monofilaments and the second plurality of multifilaments can form a second plurality of sets of filaments. Each of the first plurality of sets of filaments can include a first outer filament and a first inner filament, and each of the second plurality of sets of filaments can include a second outer filament and a second inner filament.

The first plurality of monofilaments can be thinner in diameter than the second plurality of monofilaments.

The interwoven filaments can follow a two-under/two-over configuration, where at each intersection, the second plurality of monofilaments either overlies both of the intersecting monofilaments or is overlain by both of the intersecting monofilaments and the second plurality of monofilaments overlies one of the intersecting filaments and is overlain by the other of the intersecting filaments.

The first plurality of monofilaments can have a diameter in a range of about 0.1 mm-0.4 mm. The first plurality of monofilaments can have a diameter of 0.2 mm.

The interwoven filaments can be arranged in a two-under/two-over configuration. The interwoven filaments can be arranged in a one-under/one-over configuration. The interwoven filaments can be arranged in a three-under/three-over configuration.

The orthopedic biomaterial can include a bioresorbable polymer that is configured to at least partially dissolve or be absorbed in the body. The distal end can have a distal tip with a first diameter, and the receiving portion can have a second diameter that is greater than the first diameter.

The plurality of filaments can include a plurality of flat multifilaments. The flat multifilaments can be roughened for wicking.

The filaments can outline interstices that allow for bone ingrowth, and the interstices formed by the intersecting filaments can include differently shaped and differently sized interstices.

The fastener can be a screw having a screw thread and the interior surface is configured to interact with the screw.

The distal end of the retention device can be closed.

In the first state, the interwoven filaments can extend around the tubular lattice at an angle of about 45 degrees with respect to a longitudinal direction of the retention device.

The distributed protuberances can be arranged in a diamond-shaped pattern grid.

The tubular lattice can have an outer radius spanning from a furthest outwardly extending protuberance in the radial direction on the exterior surface of the tubular lattice to a center point of the tubular lattice. The tubular lattice can have an inner radius spanning from a furthest inwardly protruding protuberance in the axial direction on the interior surface of the tubular lattice to the center point of the tubular lattice. The tubular lattice can have an average radius that is an average between the outer radius and the inner radius. The outer radius of the tubular lattice can be greatest at the intersection point of two of the thick monofilaments.

The tubular lattice can have an average diameter that is in a range of about 1.5 mm to 9.0 mm. The retention device can have a length in a range from about 30 mm to 40 mm.

When the fastener applies pressure to the interior surface, the pressure can be transmitted to protuberances on the exterior surface adjacent to the protuberance on the exterior surface and exert pressure on bone material. The retention device can further include the fastener.

Additional features, advantages, and embodiments of the invention are set forth or apparent from consideration of the following detailed description, drawings and claims. Moreover, it is to be understood that both the foregoing summary of the invention and the following detailed description are exemplary and intended to provide further explanation without limiting the scope of the invention as claimed.

In describing embodiments, specific terminology is employed for the sake of clarity. However, the invention is not intended to be limited to the specific terminology and examples selected. A person skilled in the relevant art will recognize that other equivalent components can be employed and other methods developed without departing from the broad concepts of the current invention.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A woven patch for interfacing with a bone surface, the woven patch comprising:
a sleeve body comprising a plurality of sets of interwoven filaments that form a two-dimensional lattice with a plurality of protuberances distributed on an interior surface and an exterior surface of the lattice at a predetermined spatial relationship, the plurality of sets of interwoven monofilaments having a plurality of different diameters, the sleeve body being configured to surround at least a portion of a fastener;
a first end that is configured to interface with at least a portion of the fastener; and
a second end that is opposite of the first end to the sleeve body,
wherein in a first state, the sleeve body has a plurality of combinations of filament cross-section geometries at intersection points of the interwoven filaments, the plurality of combinations of filament cross-section geometries forming a plurality of protuberance thicknesses, a thickness of each protuberance being measured in a direction as a thickness of the sleeve body, and
wherein in a second state when a fastener is inserted into or applied to the lattice, pressure from the fastener is transmitted to the lattice such that the spatial relationship of the protuberances changes according to a function of bone density and according to a function of an interfacing surface shape of the fastener.

2. The woven patch of claim 1, wherein the interwoven filaments extend across the lattice at an angle of about 45 degrees with respect to a length of the woven patch.

3. The woven patch of claim 2, wherein the distributed protuberances are arranged in a diamond-shaped pattern grid.

4. The woven patch of claim 3, wherein a length of the sleeve body is in a range from about 10 mm to 100 mm.

5. A woven retention device for interfacing with a bone surface, the woven retention device comprising:
a sleeve body comprising a plurality of filaments forming a substantially tubular lattice with a plurality of protuberances distributed on an interior surface and an exterior surface of the tubular lattice at a predetermined spatial relationship, the sleeve body being configured to surround at least a portion of a fastener, each of the plurality of protuberances being formed by an intersection point of two or more of the plurality of filaments that outline a plurality of apertures, the sleeve body comprising an orthopedic biomaterial;
a proximal end that is proximal to the sleeve body and that is configured to receive at least a portion of the fastener; and
a distal end that is distal to the sleeve body,
wherein in a first state, the sleeve body has a plurality of combinations of filament cross-section geometries at the intersection points, the plurality of combinations of filament cross-section geometries forming a plurality of protuberance thicknesses, a thickness of each protuberance being measured in a radial direction of the sleeve body, and
wherein in a second state when a fastener is inserted into the tubular lattice, pressure from the fastener is transmitted to the tubular lattice such that the spatial relationship of the protuberances changes according to a function of bone density and according to a function of an interfacing surface shape of the fastener.

6. The woven retention device of claim 5, wherein the sleeve body is configured to expand.

7. The woven retention device of claim 6, further comprising a screw-activated device including i) a screw having a threaded portion and ii) a bolt that is configured to be threaded along the threaded portion of the screw.

8. The woven retention device of claim 7, wherein a first end of the screw is attached to the distal end of the woven retention device and at least a portion of the threaded portion runs along a longitudinal direction of the body inside the woven retention device, and
wherein a second end of the screw is configured to accept the bolt such that when the bolt is moved inside the tubular structure, a compressive force is exerted on the woven retention device by the bolt in a direction parallel to a longitudinal axis of the tubular structure.

9. The woven retention device of claim 8, wherein the compressive force radially expands the tubular structure to the expanded state.

10. The woven retention device of claim 9, wherein when the fastener is inserted a predetermined distance into the tubular structure, the proximal end of the woven retention device is configured to detach from the fastener.

11. The woven retention device of claim 5, wherein the sleeve body is configured to impede biofilm formation.

12. The woven retention device of claim 11, wherein the biomaterial is made of a material that impedes biofilm formation.

13. The woven retention device of claim 11, wherein the sleeve body has a structure that impedes biofilm formation.

14. The woven retention device of claim 11, wherein the sleeve body is configured to receive a portion of the soft tissue and the sleeve body is configured to impede biofilm formation surrounding the soft tissue.

15. The woven retention device of claim 5, wherein the sleeve body comprises a coating on the plurality of filaments, wherein the coating comprises an orthopedic biomaterial.

16. The woven retention device of claim 5, wherein the plurality of filaments comprise the orthopedic biomaterial.

17. The woven retention device of claim 5, wherein the orthopedic biomaterial comprises one of PLA, PGA, PLLA, PET, PEEK, PEKK, polypropylene, polyamides, PTFE, calcium phosphate, platinum, cobalt chrome, nitinol, stainless steel, titanium, PEEK, silk and collagen, bioceramics, aluminum oxide, calcium phosphate, hydroxyapatite, glass ceramics, or any combination thereof.

18. A lattice for interfacing with a bone surface comprising:
a sleeve body comprising a plurality of filaments that form a substantially tubular lattice with a plurality of protuberances distributed on an interior surface and an exterior surface of the tubular lattice at a predetermined spatial relationship, the plurality of filaments having a plurality of different filament diameters;
a proximal end that is proximal to the sleeve body and that is configured to receive at least one of a fastener and at least a portion of a soft tissue; and
a distal end that is distal to the sleeve body,
wherein the sleeve body has a plurality of combinations of filament cross-section geometries at intersection points of the interwoven filaments, the plurality of combinations of filament cross-section geometries forming a plurality of different protuberance thicknesses, a thickness of each protuberance being measured in a radial direction of the sleeve body, and
wherein, in an implanted state of the woven retention device, the tubular lattice is configured to interface with both the soft tissue and the bone surface to secure the soft tissue to the bone surface, the spatial relationship of the protuberances changing according to a function of bone density.

19. The lattice of claim 18, wherein the sleeve body is configured to receive a portion of the soft tissue.

20. The lattice of claim 19, further comprising an anchoring device that is configured to apply pressure to one or more regions of the soft tissue, the sleeve body distributing the applied pressure through the soft tissue and the bone surface.

21. The lattice of claim 20, wherein the anchoring device penetrates the soft tissue and protrudes into the bone surface.

22. The lattice of claim 18, wherein the filaments are interwoven filaments and the interwoven filaments comprise at least one set of filament that is a felted filament.

23. The lattice of claim 18, wherein the sleeve body comprises felted filaments.

24. The lattice of claim 18, wherein the sleeve body is configured to receive a tendon.

25. The lattice of claim 18, wherein the sleeve body comprises an orthopedic biomaterial, the sleeve body being configured to minimize biofilm formation on the bone and/or soft tissue.

26. A non-transitory computer-readable storage medium having data thereon representing a three-dimensional model suitable for use in manufacturing a three-dimensional retention device for interfacing with a bone surface, the non-transitory computer-readable storage medium, when executed by at least one processor, causing a computing system to perform:
using the data in forming the three-dimensional retention device to create a plurality of filaments having input regions that interlace with other filaments,
wherein the retention device includes:
a sleeve body comprising a plurality of filaments forming a substantially tubular lattice with a plurality of protuberances distributed on an interior surface and an exterior surface of the tubular lattice at a predetermined spatial relationship, the sleeve body being configured to surround at least a portion of a fastener, each of the plurality of protuberances being formed by an intersection point of two or more of the plurality of filaments, the sleeve body including an orthopedic biomaterial;
a proximal end that is proximal to the sleeve body and that is configured to receive at least a portion of the fastener; and
a distal end that is distal to the sleeve body,
wherein in a first state, the sleeve body has a plurality of combinations of filament cross-section geometries at the intersection points, the plurality of combinations of filament cross-section geometries forming a plurality of protuberance thicknesses, a thickness of each protuberance being measured in a radial direction of the sleeve body, and
wherein in a second state when a fastener is inserted into the tubular lattice, pressure from the fastener is transmitted to the tubular lattice such that the spatial relationship of the protuberances changes according to a function of bone density and according to a function of an interfacing surface shape of the fastener.
